(19) United States
(12) Patent Application Publication
(10) Pub. No.: US 2017/0050945 A1
(43) Pub. Date: Feb. 23, 2017

(54) COMPOSITIONS AND METHODS FOR THE TREATMENT OF SARCOIDOSIS

(71) Applicant: INSMED INCORPORATED, Bridgewater, NJ (US)

(72) Inventors: Kuan-Ju CHEN, Hillsborough, NJ (US); Vladimir MALININ, Plainsboro, NJ (US); Walter PERKINS, Neshanic Station, NJ (US); Keith DIPETRILLO, Bridgewater, NJ (US); Adam PLAUNT, Bridgewater, NJ (US)

(21) Appl. No.: 15/219,219

(22) Filed: Jul. 25, 2016

Related U.S. Application Data
(60) Provisional application No. 62/196,814, filed on Jul. 24, 2015.

(51) Int. Cl. C07D 307/88; A61K 9/14; A61K 45/06; A61K 9/107; A61K 9/00; A61K 31/343; A61K 9/127

(52) U.S. Cl. CPC C07D 307/88; A61K 31/343; A61K 9/14; A61K 9/127; A61K 9/1075; A61K 9/008; A61K 9/0075; A61K 9/0078; A61K 45/06

(57) ABSTRACT

Compounds, methods, compositions and kits for treating a patient with pulmonary sarcoidosis are provided. The compositions are formulated for delivery to a patient in need of treatment via inhalation. In one embodiment, the method of treating pulmonary sarcoidosis in a patient in need thereof includes administering to the lungs of the patient via inhalation, a composition comprising an effective amount of a disease-modifying antisarcoid compound, a prodrug thereof, or a pharmaceutically acceptable salt thereof and a pharmaceutically acceptable inhalation excipient. The disease-modifying antisarcoid compound can be an immunomodulating agent, for example derivatives of mycophenolic acid, or a TNF-α antagonist.

### CROSS REFERENCE TO RELATED APPLICATIONS

The present application claims the benefit of priority to U.S. Provisional Application No. 62/196,814, filed on Jul. 24, 2015, the contents of which are hereby incorporated by reference in their entirety.

### BACKGROUND OF THE INVENTION

Sarcoidosis is an inflammatory disease characterized by abnormal masses or nodules called granulomas that may occur in many organs, such as the lung, lymph nodes, skin, eyes, liver, heart, bone and brain. The noncaseating, or non-necrotic, granulomas are small collections of modified macrophages called epithelioid cells. These collections of cells are usually encircled by lymphocytes and often contain giant cells.

Symptoms and signs of the disease are due to the granulomas altering organs and tissues. In chronic sarcoidosis cases, inflammation can eventually lead to fibrosis and permanent organ dysfunction. Sarcoidosis leads to organ damage in about one-third of the people diagnosed with the disease and may occur over many years and involve multiple organs. Sarcoidosis may also cause lupus pernio, a serious skin condition. Sarcoidosis can also be fatal. Death usually is the result of complications associated with the lungs, heart, or brain.

Sarcoidosis most often occurs in patients between 20 and 40 years of age, with women being diagnosed more frequently than men. The disease is 10 to 17 times more common in African-Americans than in Caucasians. People of Scandinavian, German, Irish, or Puerto Rican origin are also more susceptible to the disease than those of Caucasian descent. It is estimated that up to four in 10,000 people in the United States (U.S.) have sarcoidosis.

The exact cause of sarcoidosis is not known. It is a type of autoimmune disease associated with an abnormal immune response, but what triggers this response is uncertain. How sarcoidosis spreads from one part of the body to another is still being studied.

Sarcoidosis drug treatments are used to relieve symptoms and reduce the inflammation of the affected tissues. Nonsteroidal anti-inflammatory drugs (NSAIDs) are used for the treatment of arthralgias and other rheumatic complaints. For sarcoidosis involving such critical organs as the lungs, heart, liver, eyes, kidneys, or central nervous system, corticosteroid therapy has been the standard treatment.

Over 90% of people with sarcoidosis have pulmonary involvement. Also, about 20% to 50% of patients with sarcoidosis having pulmonary involvement have some permanent lung damage, and about 5 to 15% have progressive fibrosis of the lung parenchyma. At least 5% of persons will suffer pulmonary arterial hypertension. Sarcoidosis of the lung usually involves the lower respiratory tract, with inflammation of alveoli, small bronchi and small blood vessels.

Patients with pulmonary sarcoidosis are managed for the most part with synthetic glucocorticoids. Fatigue and persistent cough are usually improved with steroid treatment. However, the use of steroids is associated with debilitating side effects. Moreover, the standard therapy for serious, progressive, or life-threatening sarcoidosis that includes the administration of systemic corticosteroids is controversial. (Baltzan et al. (1999); American Journal of Respiratory and Critical Care Medicine 160, pp. 192-197). Even though corticosteroids are the standard of care, systemic corticosteroids given for periods of 6 months or longer have limited effectiveness in advanced or chronic pulmonary sarcoidosis and do not appear to alter the natural history of the disease (Baltzan et al. (1999); American Journal of Respiratory and Critical Care Medicine 160, pp. 192-197). Also, side effects with high-dose and long-term steroids are numerous and disabling in pulmonary sarcoidosis patients.

Other agents have been used to treat pulmonary sarcoidosis, but the results have varied, discerned relative to mainly small uncontrolled trials and based anecdotal evidence being reported.

Accordingly, new compounds, compositions and methods for the treatment of sarcoidosis are needed; particularly for pulmonary sarcoidosis. The present invention addresses this and other needs by providing compounds, compositions, kits and methods that provide for effective, targeted therapy of sarcoidosis in patients in need thereof.

### SUMMARY OF THE INVENTION

One aspect of the invention provides for a compound of Formula (I):

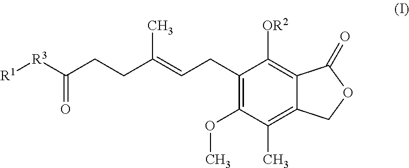

wherein
R¹ is hydrogen or C₁-C₂₀ alkyl,
R² is hydrogen, C₁-C₂₀ alkyl, or C(O)—C₁-C₁₉ alkyl, and

### R³ is NH, O, or S;

with the proviso that at least one of R¹ and R² is C₁-C₂₀ alkyl, or R² is C(O)C₁-C₁₉ alkyl, and when R² is hydrogen, and R³ is O, then R¹ is C₇₋₂₀ alkyl, or
a pharmaceutically acceptable salt thereof.

One embodiment provides for a compound of Formula (I), wherein R¹ is C₇-C₂₀ alkyl, R² is hydrogen and R³ is O, or a pharmaceutically acceptable salt thereof. Another embodiment provides for a compound of Formula (I), wherein R¹ is C₁₂ alkyl, R² is hydrogen and R³ is O, or a pharmaceutically acceptable salt thereof. Another embodiment provides for a compound of Formula (I), wherein R¹ is C₁₆ alkyl, R² is hydrogen and R³ is O, or a pharmaceutically acceptable salt thereof.

One embodiment provides for a compound of Formula (I), wherein R¹ is hydrogen, R² is C₁-C₂₀ alkyl and R³ is O, or a pharmaceutically acceptable salt thereof.

One embodiment provides for a compound of Formula (I), wherein R¹ is C₁-C₂₀ alkyl, R² is C₁-C₂₀ alkyl and R³ is O, or a pharmaceutically acceptable salt thereof.

Another aspect the invention provides for a composition comprising a pharmaceutically effective amount of the compound of Formula I, or pharmaceutically acceptable salt of the compound of Formula I, and a pharmaceutically acceptable excipient.

Yet another aspect of the invention is directed to a method of treating sarcoidosis in a patient in need thereof, comprising, administering to the patient via inhalation, a composition comprising an effective amount of a disease modifying antisarcoid compound. For example, in one embodiment, the disease modifying antisarcoid compound is represented by Formula II:

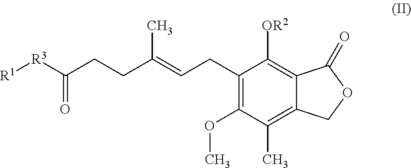

wherein:
R¹ is hydrogen or C₁-C₂₀ alkyl,
R² is hydrogen, C₁-C₂₀ alkyl, or C(O)C₁-C₁₉ alkyl, and
R³ is NH, O, or S;
or a pharmaceutically acceptable salt thereof.

In another embodiment of the method of the invention, the antisarcoid compound is a compound of Formula II, wherein R¹ and R² are hydrogen, R³ is O and the pharmaceutically acceptable salt is sodium.

In another embodiment of the method of the invention, the antisarcoid compound is a compound of Formula II, wherein R¹ is C₁ alkyl, R² is hydrogen, R³ is O, or a pharmaceutically acceptable salt thereof.

In another embodiment of the method of the invention, the antisarcoid compound is a compound of Formula II, wherein R¹ is C₁-C₂₀ alkyl, R² is hydrogen, R³ is O, or a pharmaceutically acceptable salt thereof.

In another embodiment of the method of the invention, the antisarcoid compound is a compound of Formula II, wherein R¹ is C₁-C₁₆ alkyl, R² is hydrogen, R³ is O, or a pharmaceutically acceptable salt thereof.

In another embodiment of the method of the invention, the antisarcoid compound is a compound of Formula II, wherein R¹ is C₁-C₁₀ alkyl, R² is hydrogen, R³ is O, or a pharmaceutically acceptable salt thereof.

Furthermore, another aspect of the invention is directed to a kit comprising a composition comprising a pharmaceutically effective amount of the compound of Formula I, or pharmaceutically acceptable salt of the compound of Formula I, and a pharmaceutically acceptable excipient, and an inhalation delivery device.

### BRIEF DESCRIPTION OF THE FIGURES

FIG. 1 shows the inhibition of LPS-stimulated TNF production in rat aveolar macrophages (NR8383) by mycophenolate sodium.

FIG. 2 shows the cytotoxicity of mycophenolate sodium toward rat aveolar macrophages (NR8383).

FIG. 3 shows the inhibition of LPS-stimulated TNF production in human THP-1 derived macrophages by mycophenolate sodium.

FIG. 4 shows the cytotoxicity of mycophenolate sodium toward human THP-1 derived macrophages.

FIG. 5 depicts the dose-normalized lung and plasma AUCs of mycophenolic acid following inhalation (Inh) or oral (PO) administration of mycophenolate sodium (MPS) or mycophenolate mofetil (MMF).

FIG. 6 shows the lung to plasma exposure ratios for inhaled dosing of mycophenolate sodium (MPS), mycophenolate mofetil (MMF) and hexadecyl mycophenolate (mono-C₁₆MP).

FIG. 7 shows the inhibition of LPS-stimulated TNF-α production in the lungs of C57BL/6 mice by mycophenolate sodium (MPS) and phosphate buffered saline (PBS).

FIG. 8 shows the inhibition of human inosine 5′-monophosphate dehydrogenase (IMPDH) by mycophenolic acid and hexadecyl mycophenolate (mono-C₁₆MP).

### DETAILED DESCRIPTION OF THE INVENTION

The term “disease-modifying antisarcoid compound” refers to a compound, a derivative thereof (e.g., a prodrug thereof), metabolite thereof, or a pharmaceutically acceptable salt thereof, that is used to treat a patient with pulmonary sarcoidosis. These compounds can include, but are not limited to, potentiators of glucocorticosteroids, cytotoxic compounds, steroid-sparing compounds, immunomodulating compounds and immunosuppressive agents. The disease-modifying antisarcoid compound can be a biologic, such as an antibody or nucleic acid. For example, as described herein, an anti-tumor necrosis factor alpha (TNF-α) antibody is one embodiment of a disease-modifying antisarcoid compound.

Reference to a “disease-modifying antisarcoid compound” includes the compound, a derivative of the compound (e.g., a prodrug, metabolite or conjugate), a pharmaceutically acceptable salt of the compound, or a pharmaceutically acceptable salt of the derivative (e.g., a pharmaceutically acceptable salt of a prodrug).

The term “prodrug” or “pharmaceutically acceptable prodrug,” as used herein refers to a compound that is transformed in vivo to yield the parent compound, for example by hydrolysis. As used herein, the term “prodrug” is encompassed by the term “derivative.” Effective dosages of the disease-modifying antisarcoid compound can be the same as those previously reported for the particular compound, and also modified according to ordinary skill in the art.

The term “pharmaceutical” or “pharmaceutically acceptable” when used herein as an adjective, means substantially non-toxic and substantially non-deleterious to the recipient. As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Effective amount” or “therapeutically effective amount” means an amount of disease-modifying antisarcoid compound, a prodrug thereof, or a pharmaceutically acceptable salt thereof, used in the present invention sufficient to result in the desired therapeutic response.

The term “treating” includes: (1) preventing or delaying the appearance of clinical symptoms of the state, disorder or condition developing in the subject that may be afflicted with or predisposed to the state, disorder or condition but does not yet experience or display clinical or subclinical symptoms of the state, disorder or condition; (2) inhibiting the state, disorder or condition (i.e., arresting, reducing or delaying the development of the disease, or a relapse thereof in case of maintenance treatment, of at least one clinical or subclinical symptom thereof); and/or (3) relieving the condition (e.g., causing regression of the state, disorder or condition or at least one of its clinical or subclinical symptoms). The benefit to a subject to be treated is either statistically significant or at least perceptible to the subject or to the physician.

“Prophylaxis,” as used herein, can mean complete prevention of an infection or disease, or prevention of the development of symptoms of that infection or disease; a delay in the onset of an infection or disease or its symptoms; or a decrease in the severity of a subsequently developed infection or disease or its symptoms.

The term “subject” as used herein, refers to an animal, for example a mammal. In one embodiment, the subject is a human. Non-limiting examples of subjects treatable with the methods, compositions and kits described herein include a human, primate, cow, horse, sheep, goat, dog, cat, rabbit and a rodent. The term “subject” may be interchangeably used with the term patient in the context of the present invention.

The term “salt” or “salts” as used herein encompasses pharmaceutically acceptable salts commonly used to form alkali metal salts of free acids and to form addition salts of free bases. The nature of the salt is not critical, provided that it is pharmaceutically acceptable. Suitable pharmaceutically acceptable acid addition salts may be prepared from an inorganic acid or from an organic acid. Exemplary pharmaceutical salts are disclosed in Stahl, P. H., Wermuth, C. G., Eds. Handbook of Pharmaceutical Salts: Properties, Selection and Use; Verlag Helvetica Chimica Acta/Wiley-VCH: Zurich, 2002, the contents of which are hereby incorporated by reference in their entirety. Specific non-limiting examples of inorganic acids are hydrochloric, hydrobromic, hydroiodic, nitric, carbonic, sulfuric and phosphoric acid. Appropriate organic acids include, without limitation, aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric or galacturonic acid. Suitable pharmaceutically acceptable salts of free acid-containing compounds disclosed herein include, without limitation, metallic salts and organic salts. Exemplary metallic salts include, but are not limited to, appropriate alkali metal (group Ia) salts, alkaline earth metal (group IIa) salts, and other physiological acceptable metals. Such salts can be made from aluminum, calcium, lithium, magnesium, potassium, sodium and zinc; more particularly potassium or sodium; further particularly sodium. Exemplary organic salts can be made from primary amines, secondary amines, tertiary amines and quaternary ammonium salts, for example, tromethamine, diethylamine, tetra-N-methylammonium, N,N′-dibenzylethylenediamine, chloroprocaine, choline, diethanolamine, ethylenediamine, meglumine (N-methylglucamine) and procaine.

“Alkyl” or “alkyl group” refers to a fully saturated, straight or branched hydrocarbon chain radical having from one to twenty carbon atoms, and which is attached to the rest of the molecule by a single bond. Alkyls comprising any number of carbon atoms from 1 to 20 are included. An alkyl comprising up to 20 carbon atoms is a C₁-C₂₀ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₂₀ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, n-dodecyl, n-tridecanyl, n-tetradecyl, n-pentadecyl, n-hexadecyl, n-heptadecyl, n-octadecyl, n-nonadecyl and n-icosanyl. Unless stated otherwise specifically in the specification, an alkyl group can be optionally substituted and/or unsaturated. The term unsaturated used herein means an alkyl may have one or more double or triple bonds between adjacent carbon atoms, i.e., alkyl includes corresponding alkenyl or alkynyl group.

The term “substituted” used herein relative to any one of the alkyl, alkyenyl, alkynyl, cycloalkyl, cyclalkenyl, cycloalkynyl, heterocyclyl, aryl and heteroaryl groups means wherein at least one hydrogen atom of the group is replaced by a substituent selected from group consisting of halo, cycloalkyl, cycloalkenyl, heterocyclyl, aryl, heteroaryl, —CN, —NO₂, ═O, ═S, ═NR_{g}, —NR_{g}Rₕ, —NR_{g}C(═O)Rₕ, —NR_{g}C(═O)NR_{g}Rₕ, —NR_{g}C(═O)ORₕ, —NR_{g} SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, —SO₂NR_{g}Rₕ, —C(═O)R_{g}, —C(═O)OR_{g}, and —C(═O)NR_{g}Rₕ group, wherein R_{g} and Rₕ are the same or different and independently selected from the group of hydrogen, alkyl, aryl, cycloalkyl, cycloalkenyl, heterocyclyl, and heteroaryl. In addition, each of the foregoing substituents can also be optionally substituted with one or more of the above substituents.

“Alkenyl” or “alkenyl group” refers to a straight or branched hydrocarbon chain radical having from two to twenty carbon atoms, and having one or more carbon-carbon triple bonds. Each alkenyl group is attached to the rest of the molecule by a single bond. Alkenyl group comprising any number of carbon atoms from 2 to 20 are included. An alkenyl group comprising up to 20 carbon atoms is a C₂-C₂₀ alkenyl, an alkenyl comprising up to 10 carbon atoms is a C₂-C₁₀ an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyl. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₂₀ alkenyl include ethenyl, propenyl, butenyl, pentenyl, n-hexenyl, n-heptenyl, n-octenyl, n-nonenyl, n-decenyl, n-undecenyl, n-dodecenyl, n-tridecanenyl, n-tetradecenyl, n-pentadecenyl, n-hexadecenyl, n-heptadecenyl, n-octadecenyl, n-nonadecenyl and n-icosanenyl and the like. Unless stated otherwise specifically in the specification, an alkenyl group can be optionally substituted.

“Alkynyl” or “alkynyl group” refers to a straight or branched hydrocarbon chain radical having from two to twenty carbon atoms, and having one or more carbon-carbon triple bonds. Each alkynyl group is attached to the rest of the molecule by a single bond. Alkynyl group comprising any number of carbon atoms from 2 to 20 are included. An alkynyl group comprising up to 20 carbon atoms is a C₂-C₂₀ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyl. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₂₀ alkynyl include ethynyl, propynyl, butynyl, pentynyl, n-hexynyl, n-heptynyl, n-octynyl, n-nonynyl, n-decynyl, n-undecynyl, n-dodecynyl, n-tridecanynyl, n-tetradecynyl, n-pentadecynyl, n-hexadecynyl, n-heptadecynyl, n-octadecynyl, n-nonadecynyl and n-icosanynyl and the like. Unless stated otherwise specifically in the specification, an alkynyl group can be optionally substituted.

“Alkoxy” refers to a radical of the formula —ORₐ where Rₐ is an alkyl, alkenyl or alknyl radical as defined above containing up to twelve carbon atoms. Unless stated otherwise specifically in the specification, an alkoxy group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyl radicals include, for example, cyclopropenyl, cyclbutenyl, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from eight to twenty carbon atoms, preferably having from eight to ten carbon atoms, and which is attached to the rest of the molecule by a single bond. Monocyclic cycloalkynyl radicals include, for example, cyclooctynyl, and the like. Unless otherwise stated specifically in the specification, a cycloalkynyl group can be optionally substituted.

“Heterocyclyl,” “heterocyclic ring” or “heterocycle” refers to a stable 3- to 20-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heteroaryl” refers to a 5- to 20-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

“Thioalkyl” refers to a radical of the formula —SRₐ where Rₐ is an alkyl, alkenyl, or alkynyl radical as defined above containing up to twelve carbon atoms. Unless stated otherwise specifically in the specification, a thioalkyl group can be optionally substituted.

One aspect of the invention provides a compound of Formula (I)

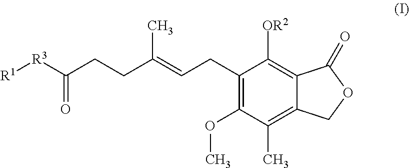

wherein
R¹ is hydrogen or C₁-C₂₀ alkyl,
R² is hydrogen, C₁-C₂₀ alkyl, or C(O)C₁-C₁₉ alkyl, and

### R³ is NH, O, or S;

with the proviso that at least one of R¹ and R² is C₁-C₂₀ alkyl, or R² is C(O)C₁-C₁₉ alkyl, and when R² is hydrogen, and R³ is O, then R¹ is C₇₋₂₀ alkyl, or
a pharmaceutically acceptable salt thereof.

In one embodiment of Formula (I), wherein R³ is O, or a pharmaceutically acceptable salt thereof.

In one embodiment, a compound of Formula (Ia) is provided

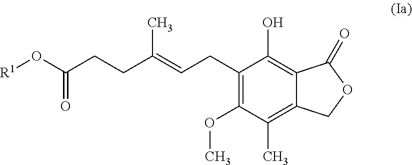

wherein R¹ is C₇-C₂₀ alkyl, or a pharmaceutically acceptable salt thereof. In another embodiment of Formula (Ia), R¹ is C₇-C₁₈. In another embodiment of Formula (Ia), R¹ is C₇-C₁₆. In another embodiment of Formula (Ia), R¹ is C₇-C₁₄. In another embodiment of Formula (Ia), R¹ is C₇-C₁₂. In another embodiment of Formula (Ia), R¹ is C₇-C₁₀.

In one embodiment of Formula (Ia), R¹ is C₈-C₂₀ alkyl. In another embodiment of Formula (Ia), R¹ is C₈-C₁₆ alkyl.

In one embodiment of Formula (Ia), R¹ is C₉-C₂₀ alkyl.

In one embodiment of Formula (Ia), R¹ is C₁₀-C₂₀ alkyl. In another embodiment of Formula (Ia), R¹ is C₁₀-C₁₆ alkyl.

In one embodiment of Formula (Ia), R¹ is C₁₂-C₂₀ alkyl. In another embodiment of Formula (Ia), R¹ is C₁₂-C₁₈ alkyl. In another embodiment of Formula (Ia), R¹ is C₁₂-C₁₆ alkyl.

In one embodiment of Formula (Ia), R¹ is C₁₄-C₂₀ alkyl. In another embodiment of Formula (Ia), R¹ is C₁₄-C₁₆ alkyl.

In one embodiment of Formula (Ia), R¹ is C₁₆-C₂₀ alkyl.

In one embodiment of Formula (Ia), R¹ is C₁₈-C₂₀ alkyl.

In one embodiment of Formula (Ia), R¹ is C₁₂ alkyl.

In one embodiment of Formula (Ia), R¹ is C₁₃ alkyl.

In one embodiment of Formula (Ia), R¹ is C₁₄ alkyl.

In one embodiment of Formula (Ia), R¹ is C₁₅ alkyl.

In one embodiment of Formula (Ia), R¹ is C₁₆ alkyl.

In one embodiment of Formula (Ia), R¹ is C₁₇ alkyl.

In one embodiment of Formula (Ia), R¹ is C₁₈ alkyl.

In one embodiment of Formula (Ia), R¹ is C₁₉ alkyl.

In one embodiment of Formula (Ia), R¹ is C₂₀ alkyl.

In another embodiment, a compound of Formula (Ib) is provided

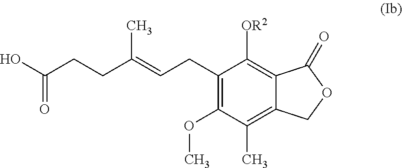

wherein R² is C₁-C₂₀ alkyl or C(O)C₁-C₁₉ alkyl, or a pharmaceutically acceptable salt thereof.

In one embodiment of Formula (Ib), R² is C₁-C₂₀ alkyl. In another embodiment of Formula (Ib), R² is C₇-C₂₀ alkyl. In yet another embodiment of Formula (Ib), R² is C₉-C₂₀ alkyl. In another further embodiment of Formula (Ib), R² is C₁₂-C₂₀ alkyl.

In one embodiment of Formula (Ib), R² is C₁₂ alkyl.

In one embodiment of Formula (Ib), R² is C₁₃ alkyl.

In one embodiment of Formula (Ib), R² is C₁₄ alkyl.

In one embodiment of Formula (Ib), R² is C₁₅ alkyl.

In one embodiment of Formula (Ib), R² is C₁₆ alkyl.

In one embodiment of Formula (Ib), R² is C₁₇ alkyl.

In one embodiment of Formula (Ib), R² is C₁₈ alkyl.

In one embodiment of Formula (Ib), R² is C₁₉ alkyl.

In one embodiment of Formula (Ib), R² is C₂₀ alkyl.

In each of the embodiments of Formula (I) where R¹ is hydrogen and the embodiments of Formula (Ib), the embodiment includes pharmaceutically acceptable salts thereof. In additional embodiments, the pharmaceutically acceptable salt is sodium or potassium. In another embodiment, the pharmaceutically acceptable salt is sodium.

In another embodiment, a compound of Formula (Ic) is provided

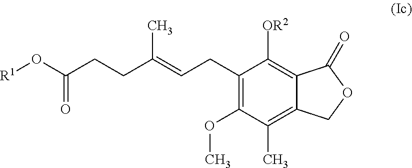

wherein R¹ is C₁-C₂₀ alkyl and R² is C₁-C₂₀ alkyl or C(O)C₁-C₁₉ alkyl, or a pharmaceutically acceptable salt thereof. In a further embodiment, R¹ is C₁-C₂₀ alkyl and R² is C₁-C₂₀ alkyl. In a further embodiment, R¹ is C₁-C₂₀ alkyl, and R² is C(O)C₁-C₁₉ alkyl.

In one embodiment of Formula (Ic), R¹ is C₁-C₂₀ alkyl and R² is C₁-C₂₀ alkyl. In another embodiment of Formula (Ic), R¹ is C₁-C₁₅ alkyl and R² is C₁-C₁₅ alkyl. In another embodiment of Formula (Ic), R¹ is C₁-C₁₃ alkyl and R² is C₁-C₁₃ alkyl. In another embodiment of Formula (Ic), R¹ is C₁-C₁₁ alkyl and R² is C₁-C₁₁ alkyl. In another embodiment of Formula (Ic), R¹ is C₁-C₉ alkyl and R² is C₁-C₉ alkyl. In another embodiment of Formula (Ic), R¹ is C₁-C₇ alkyl and R² is C₁-C₇ alkyl. In another embodiment of Formula (Ic), R¹ is C₁-C₅ alkyl and R² is C₁-C₅ alkyl. In another embodiment of Formula (Ic), R¹ is C₁-C₃ alkyl and R² is C₁-C₃ alkyl.

In one embodiment of Formula (Ic), R¹ is C₃-C₁₄ alkyl and R² is C₃-C₁₄ alkyl. In another embodiment of Formula (Ic), R¹ is C₃-C₉ alkyl and R² is C₃-C₉ alkyl. In another embodiment of Formula (Ic), R¹ is C₃-C₅ alkyl and R² is C₃-C₅ alkyl.

In one embodiment of Formula (Ic), R¹ is C₅-C₁₃ alkyl and R² is C₅-C₁₃ alkyl. In another embodiment of Formula (Ic), R¹ is C₅-C₉ alkyl and R² is C₅-C₉ alkyl. In another embodiment of Formula (Ic), R¹ is C₅-C₇ alkyl and R² is C₅-C₇ alkyl.

In one embodiment of Formula (Ic), R¹ is C₆-C₂₀ alkyl and R² is C₆-C₂₀ alkyl. In another embodiment of Formula (Ic), R¹ is C₆-C₈ alkyl and R² is C₆-C₈ alkyl.

In another embodiment of Formula (Ic), R¹ is C₇-C₂₀ alkyl and R² is C₇-C₂₀ alkyl.

In one embodiment of Formula (Ic), R¹ is C₈-C₂₀ alkyl and R² is C₈-C₂₀ alkyl. In another embodiment of Formula (Ic), R¹ is C₈-C₁₆ alkyl and R² is C₈-C₁₆ alkyl.

In another embodiment of Formula (Ic), R¹ is C₉-C₂₀ alkyl and R² is C₉-C₂₀ alkyl.

In one embodiment of Formula (Ic), R¹ is C₁₀-C₂₀ alkyl and R² is C₁₀-C₂₀ alkyl. In another embodiment of Formula (Ic), R¹ is C₁₀-C₁₆ alkyl and R² is C₁₀-C₁₆ alkyl.

In one embodiment of Formula (Ic), R¹ is C₁₂-C₂₀ alkyl and R² is C₁₂-C₂₀ alkyl. In another embodiment of Formula (Ic), R¹ is C₁₂-C₁₈ alkyl and R² is C₁₂-C₁₈ alkyl. In another embodiment of Formula (Ic), R¹ is C₁₂-C₁₆ alkyl and R² is C₁₂-C₁₆ alkyl.

In one embodiment of Formula (Ic), R¹ is C₁₄-C₂₀ alkyl and R² is C₁₄-C₂₀ alkyl. In another embodiment of Formula (Ic), R¹ is C₁₄-C₁₆ alkyl and R² is C₁₄-C₁₆ alkyl.

In one embodiment of Formula (Ic), R¹ is C₁₆-C₂₀ alkyl and R² is C₁₆-C₂₀ alkyl.

In one embodiment of Formula (Ic), R¹ is C₁₈-C₂₀ alkyl and R² is C₁₈-C₂₀ alkyl

In one embodiment of Formula (Ic), R¹ is C₁₂ alkyl and R² is C₁₂ alkyl.

In one embodiment of Formula (Ic), R¹ is C₁₃ alkyl and R² is C₁₃ alkyl.

In one embodiment of Formula (Ic), R¹ is C₁₄ alkyl and R² is C₁₄ alkyl.

In one embodiment of Formula (Ic), R¹ is C₁₅ alkyl and R² is C₁₅ alkyl.

In one embodiment of Formula (Ic), R¹ is C₁₆ alkyl and R² is C₁₆ alkyl.

In one embodiment of Formula (Ic), R¹ is C₁₇ alkyl and R² is C₁₇ alkyl.

In one embodiment of Formula (Ic), R¹ is C₁₈ alkyl and R² is C₁₈ alkyl.

In one embodiment of Formula (Ic), R¹ is C₁₉ alkyl and R² is C₁₉ alkyl.

In one embodiment of Formula (Ic), R¹ is C₂₀ alkyl and R² is C₂₀ alkyl.

In one embodiment, a compound of Formula (Id) is provided

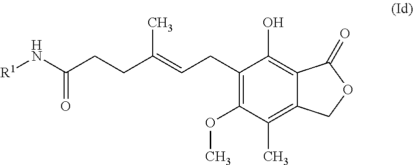

wherein R¹ is C₁-C₂₀ alkyl, or a pharmaceutically acceptable salt thereof.

In another embodiment, a compound of Formula (Ie) is provided

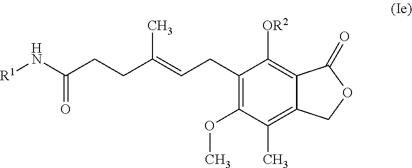

wherein R¹ is C₁-C₂₀ alkyl and R² is C₁-C₂₀ alkyl or C(O)C₁-C₁₉ alkyl, or a pharmaceutically acceptable salt thereof. In a further embodiment, R¹ is C₁-C₂₀ alkyl and R² is C₁-C₂₀ alkyl, or a pharmaceutically acceptable salt thereof. In a further embodiment, R¹ is C₁-C₂₀ alkyl and R² is C(O)C₁-C₁₉ alkyl, or a pharmaceutically acceptable salt thereof.

In one embodiment, a compound of Formula (If) is provided

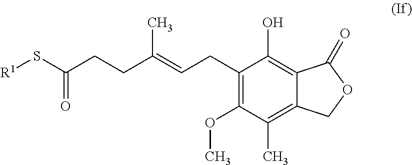

wherein R¹ is C₁-C₂₀ alkyl, or a pharmaceutically acceptable salt thereof.

In one embodiment, a compound of Formula (Ig) is provided

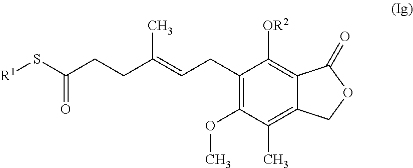

wherein R¹ is C₁-C₂₀ alkyl and R² is C₁-C₂₀ alkyl or C(O)C₁-C₁₉ alkyl, or a pharmaceutically acceptable salt thereof. In a further embodiment, R¹ is C₁-C₂₀ alkyl and R² is C₁-C₂₀ alkyl, or a pharmaceutically acceptable salt thereof. In a further embodiment, R¹ is C₁-C₂₀ alkyl and R² is C(O)C₁-C₁₉ alkyl, or a pharmaceutically acceptable salt thereof.

The invention also provides for a composition comprising a pharmaceutically effective amount of the compound of Formula I, or pharmaceutically acceptable salt of the compound of Formula I, and a pharmaceutically acceptable excipient.

The composition embodiments of the invention also is directed to each of the compound embodiments noted above regarding Formula (I), i.e., Formula Ia, Ib, Ic, Id, Ie, If or Ig.

Another embodiment of the invention is directed to a composition comprising an effective amount of a compound of Formula (I), wherein R¹ is C₁-C₂₀ alkyl, R² is C₁-C₂₀ alkyl, and R³ is O.

Another embodiment of the invention is directed to a composition comprising an effective amount of a compound of Formula (I), wherein R¹ is C₁₂ alkyl, R² is hydrogen and R³ is O.

Another embodiment of the invention is directed to a composition comprising an effective amount of a compound of Formula (I), wherein R¹ is C₁₆ alkyl, R² is hydrogen and R³ is O.

Another embodiment of the invention is directed to a composition comprising an effective amount of a compound of Formula (I), wherein R¹ is C₁₂ alkyl, R² is C₁₂ alkyl and R³ is O.

Another embodiment of the invention is directed to a composition comprising an effective amount of a compound of Formula (I), wherein R¹ is C₁₆ alkyl, R² is C₁₆ alkyl and R³ is O.

Another aspect of the present invention provides for a composition comprising an effective amount of a disease-modifying antisarcoid compound, a derivative thereof (e.g., a prodrug thereof), or a pharmaceutically acceptable salt thereof, and a pharmaceutically acceptable inhalation excipient.

In another aspect of the invention, a method of treating sarcoidosis is provided. The method comprises administering to a patient in need of sarcoidosis treatment, via inhalation, a composition comprising an effective amount of a disease modifying antisarcoid compound.

In one embodiment, the disease modifying antisarcoid compound is a compound of Formula I, as discussed above. In a further embodiment, the compound of Formula (I) is a compound of Formula Ia, Ib, Ic, Id, Ie, If or Ig, as discussed in greater detail above.

In one embodiment, the disease modifying antisarcoid compound is a compound of Formula II:

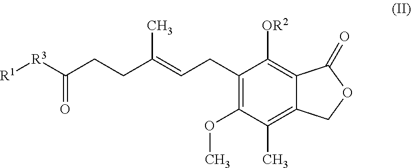

wherein:
R¹ is hydrogen or C₁-C₂₀ alkyl,
R² is hydrogen, C₁-C₂₀ alkyl, or C(O)C₁-C₁₉ alkyl, and
R³ is NH, O, or S;
or a pharmaceutically acceptable salt thereof.

In a further embodiment, the composition administered via the method provided herein comprises an effective amount of a compound of Formula (II), wherein and R² are hydrogen and R³ is O, or a pharmaceutically acceptable salt thereof.

In one embodiment, the composition administered via the method provided herein comprises an effective amount of a compound of Formula (II), wherein R¹ is C₁-C₂₀ alkyl, R² is hydrogen and R³ is O, or a pharmaceutically acceptable salt thereof.

In one embodiment, the composition administered via the method provided herein comprises an effective amount of a compound of Formula (II), wherein R¹ is C₁-C₁₅ alkyl, R² is hydrogen and R³ is O, or a pharmaceutically acceptable salt thereof.

In one embodiment, the composition administered via the method provided herein comprises an effective amount of a compound of Formula (II), wherein R¹ is C₁-C₁₃ alkyl, R² is hydrogen and R³ is O, or a pharmaceutically acceptable salt thereof.

In one embodiment, the composition administered via the method provided herein comprises an effective amount of a compound of Formula (II), wherein R¹ is C₁-C₁₁ alkyl, R² is hydrogen and R³ is O, or a pharmaceutically acceptable salt thereof.

In one embodiment, the composition administered via the method provided herein comprises an effective amount of a compound of Formula (II), wherein R¹ is C₁-C₉ alkyl, R² is hydrogen and R³ is O, or a pharmaceutically acceptable salt thereof.

In one embodiment, the composition administered via the method provided herein comprises an effective amount of a compound of Formula (II), wherein R¹ is C₁-C₇ alkyl, R² is hydrogen and R³ is O, or a pharmaceutically acceptable salt thereof.

In one embodiment, the composition administered via the method provided herein comprises an effective amount of a compound of Formula (II), wherein R¹ is C₁-C₅ alkyl, R² is hydrogen and R³ is O, or a pharmaceutically acceptable salt thereof.

In one embodiment, the composition administered via the method provided herein comprises an effective amount of a compound of Formula (II), wherein R¹ is C₁-C₃ alkyl, R² is hydrogen and R³ is O, or a pharmaceutically acceptable salt thereof.

In one embodiment, the composition administered via the method provided herein comprises an effective amount of a compound of Formula (II), wherein R¹ is C₃-C₁₄ alkyl, R² is hydrogen and R³ is O, or a pharmaceutically acceptable salt thereof.

In one embodiment, the composition administered via the method provided herein comprises an effective amount of a compound of Formula (II), wherein R¹ is C₃-C₉ alkyl, R² is hydrogen and R³ is O, or a pharmaceutically acceptable salt thereof.

In one embodiment, the composition administered via the method provided herein comprises an effective amount of a compound of Formula (II), wherein R¹ is C₃-C₅ alkyl, R² is hydrogen and R³ is O, or a pharmaceutically acceptable salt thereof.

In one embodiment, the composition administered via the method provided herein comprises an effective amount of a compound of Formula (II), wherein R¹ is C₅-C₁₃ alkyl, R² is hydrogen and R³ is O, or a pharmaceutically acceptable salt thereof.

In one embodiment, the composition administered via the method provided herein comprises an effective amount of a compound of Formula (II), wherein R¹ is C₅-C₉ alkyl, R² is hydrogen and R³ is O, or a pharmaceutically acceptable salt thereof.

In one embodiment, the composition administered via the method provided herein comprises an effective amount of a compound of Formula (II), wherein R¹ is C₅-C₇ alkyl, R² is hydrogen and R³ is O, or a pharmaceutically acceptable salt thereof.

In one embodiment, the composition administered via the method provided herein comprises an effective amount of a compound of Formula (II), wherein R¹ is C₆-C₂₀ alkyl, R² is hydrogen and R³ is O, or a pharmaceutically acceptable salt thereof.

In one embodiment, the composition administered via the method provided herein comprises an effective amount of a compound of Formula (II), wherein R¹ is C₆-C₈ alkyl, R² is hydrogen and R³ is O, or a pharmaceutically acceptable salt thereof.

In a one embodiment, the composition administered via the method provided herein comprises an effective amount of a compound of Formula (II), wherein R¹ is C₇-C₂₀ alkyl, R² is hydrogen and R³ is O, or a pharmaceutically acceptable salt thereof.

In one embodiment, the composition administered via the method provided herein comprises an effective amount of a compound of Formula (II), wherein R¹ is C₈-C₂₀ alkyl, R² is hydrogen and R³ is O, or a pharmaceutically acceptable salt thereof.

In one embodiment, the composition administered via the method provided herein comprises an effective amount of a compound of Formula (II), wherein R¹ is C₈-C₁₆ alkyl, R² is hydrogen and R³ is O, or a pharmaceutically acceptable salt thereof.

In another embodiment, the composition administered via the method provided herein comprises an effective amount of a compound of Formula (II), wherein R¹ is C₉-C₂₀ alkyl, R² is hydrogen and R³ is O, or a pharmaceutically acceptable salt thereof.

In one embodiment, the composition administered via the method provided herein comprises an effective amount of a compound of Formula (II), wherein R¹ is C₁₀-C₂₀ alkyl, R² is hydrogen and R³ is O, or a pharmaceutically acceptable salt thereof.

In one embodiment, the composition administered via the method provided herein comprises an effective amount of a compound of Formula (II), wherein R¹ is C₁₀-C₁₆ alkyl, R² is hydrogen and R³ is O, or a pharmaceutically acceptable salt thereof.

In yet another embodiment, the composition administered via the method provided herein comprises an effective amount of a compound of Formula (II), wherein R¹ is C₁₂-C₂₀ alkyl, R² is hydrogen and R³ is O, or a pharmaceutically acceptable salt thereof.

In one embodiment, the composition administered via the method provided herein comprises an effective amount of a compound of Formula (II), wherein R¹ is C₁₂-C₁₈ alkyl, R² is hydrogen and R³ is O, or a pharmaceutically acceptable salt thereof.

In one embodiment, the composition administered via the method provided herein comprises an effective amount of a compound of Formula (II), wherein R¹ is C₁₂-C₁₆ alkyl, R² is hydrogen and R³ is O, or a pharmaceutically acceptable salt thereof.

In one embodiment, the composition administered via the method provided herein comprises an effective amount of a compound of Formula (II), wherein R¹ is C₁₄-C₂₀ alkyl, R² is hydrogen and R³ is O, or a pharmaceutically acceptable salt thereof.

In one embodiment, the composition administered via the method provided herein comprises an effective amount of a compound of Formula (II), wherein R¹ is C₁₄-C₁₆ alkyl, R² is hydrogen and R³ is O, or a pharmaceutically acceptable salt thereof.

In one embodiment, the composition administered via the method provided herein comprises an effective amount of a compound of Formula (II), wherein R¹ is C₁₆-C₂₀ alkyl, R² is hydrogen and R³ is O, or a pharmaceutically acceptable salt thereof.

In one embodiment, the composition administered via the method provided herein comprises an effective amount of a compound of Formula (II), wherein R¹ is C₁₈-C₂₀ alkyl, R² is hydrogen and R³ is O, or a pharmaceutically acceptable salt thereof.

In a further embodiment, the composition administered via the method provided herein comprises an effective amount of a compound of Formula (II), wherein R¹ is C₁₂ alkyl, R² is hydrogen and R³ is O.

In a further embodiment, the composition administered via the method provided herein comprises an effective amount of a compound of Formula (II), wherein R¹ is C₁₃ alkyl, R² is hydrogen and R³ is O.

In a preferred embodiment, the composition administered via the method provided herein comprises an effective amount of a compound of Formula (II), wherein R¹ is C₁₄ alkyl, R² is hydrogen and R³ is O.

In a further embodiment, the composition administered via the method provided herein comprises an effective amount of a compound of Formula (II), wherein R¹ is C₁₅ alkyl, R² is hydrogen and R³ is O.

In a preferred embodiment, the composition administered via the method provided herein comprises an effective amount of a compound of Formula (II), wherein R¹ is C₁₆ alkyl, R² is hydrogen and R³ is O.

In a further embodiment, the composition administered via the method provided herein comprises an effective amount of a compound of Formula (II), wherein R¹ is C₁₇ alkyl, R² is hydrogen and R³ is O.

In a preferred embodiment, the composition administered via the method provided herein comprises an effective amount of a compound of Formula (II), wherein R¹ is C₁₈ alkyl, R² is hydrogen and R³ is O.

In a further embodiment, the composition administered via the method provided herein comprises an effective amount of a compound of Formula (II), wherein R¹ is C₁₉ alkyl, R² is hydrogen and R³ is O.

In a preferred embodiment, the composition administered via the method provided herein comprises an effective amount of a compound of Formula (II), wherein R¹ is C₂₀ alkyl, R² is hydrogen and R³ is O.

In a further embodiment, the composition administered via the method provided herein comprises an effective amount of a compound of Formula (II), wherein R¹ is hydrogen, R² is C₁-C₂₀ alkyl and R³ is O, or a pharmaceutically acceptable salt thereof.

In a preferred embodiment, the composition administered via the method provided herein comprises an effective amount of a compound of Formula (II), wherein R¹ is hydrogen, R² is C₇-C₂₀ alkyl and R³ is O, or a pharmaceutically acceptable salt thereof.

In a preferred embodiment, the composition administered via the method provided herein comprises an effective amount of a compound of Formula (II), wherein R¹ is hydrogen, R² is C₉-C₂₀ alkyl and R³ is O, or a pharmaceutically acceptable salt thereof.

In a preferred embodiment, the composition administered via the method provided herein comprises an effective amount of a compound of Formula (II), wherein R¹ is hydrogen, R² is C₁₂-C₂₀ alkyl and R³ is O, or a pharmaceutically acceptable salt thereof.

In a preferred embodiment, the composition administered via the method provided herein comprises an effective amount of a compound of Formula (II), wherein R¹ is hydrogen, R² is C₁₂ alkyl and R³ is O, or a pharmaceutically acceptable salt thereof.

In a further embodiment, the composition administered via the method provided herein comprises an effective amount of a compound of Formula (II), wherein R¹ is hydrogen, R² is C₁₃ alkyl and R³ is O, or a pharmaceutically acceptable salt thereof.

In a further embodiment, the composition administered via the method provided herein comprises an effective amount of a compound of Formula (II), wherein R¹ is hydrogen, R² is C₁₄ alkyl and R³ is O, or a pharmaceutically acceptable salt thereof.

In a further embodiment, the composition administered via the method provided herein comprises an effective amount of a compound of Formula (II), wherein R¹ is hydrogen, R² is C₁₅ alkyl and R³ is O, or a pharmaceutically acceptable salt thereof.

In a preferred embodiment, the composition administered via the method provided herein comprises an effective amount of a compound of Formula (II), wherein R¹ is hydrogen, R² is C₁₆ alkyl and R³ is O, or a pharmaceutically acceptable salt thereof.

In a further embodiment, the composition administered via the method provided herein comprises an effective amount of a compound of Formula (II), wherein R¹ is hydrogen, R² is C₁₇ alkyl and R³ is O, or a pharmaceutically acceptable salt thereof.

In a preferred embodiment, the composition administered via the method provided herein comprises an effective amount of a compound of Formula (II), wherein R¹ is hydrogen, R² is C₁₈ alkyl and R³ is O, or a pharmaceutically acceptable salt thereof.

In a further embodiment, the composition administered via the method provided herein comprises an effective amount of a compound of Formula (II), wherein R¹ is hydrogen, R² is C₁₉ alkyl and R³ is O, or a pharmaceutically acceptable salt thereof.

In a preferred embodiment, the composition administered via the method provided herein comprises an effective amount of a compound of Formula (II), wherein R¹ is hydrogen, R² is C₂₀ alkyl and R³ is O, or a pharmaceutically acceptable salt thereof.

In a preferred embodiment, the composition administered via the method provided herein comprises an effective amount of a compound of Formula (II), wherein R¹ is C₁-C₂₀ alkyl, R² is C₁-C₂₀ alkyl and R³ is O, or a pharmaceutically acceptable salt thereof.

In another embodiment, the composition administered via the method provided herein comprises an effective amount of a compound of Formula (II), wherein R¹ is C₁-C₁₅ alkyl, R² is C₁-C₁₅ alkyl and R³ is O, or a pharmaceutically acceptable salt thereof.

In another embodiment, the composition administered via the method provided herein comprises an effective amount of a compound of Formula (II), wherein R¹ is C₁-C₁₃ alkyl, R² is C₁-C₁₃ alkyl and R³ is O, or a pharmaceutically acceptable salt thereof.

In another embodiment, the composition administered via the method provided herein comprises an effective amount of a compound of Formula (II), wherein R¹ is C₁-C₁₁ alkyl, R² is C₁-C₁₁ alkyl and R³ is O, or a pharmaceutically acceptable salt thereof.

In another embodiment, the composition administered via the method provided herein comprises an effective amount of a compound of Formula (II), wherein R¹ is C₁-C₉ alkyl, R² is C₁-C₉ alkyl and R³ is O, or a pharmaceutically acceptable salt thereof.

In another embodiment, the composition administered via the method provided herein comprises an effective amount of a compound of Formula (II), wherein R¹ is C₁-C₇ alkyl, R² is C₁-C₇ alkyl and R³ is O, or a pharmaceutically acceptable salt thereof.

In another embodiment, the composition administered via the method provided herein comprises an effective amount of a compound of Formula (II), wherein R¹ is C₁-C₅ alkyl, R² is C₁-C₅ alkyl and R³ is O, or a pharmaceutically acceptable salt thereof.

In another embodiment, the composition administered via the method provided herein comprises an effective amount of a compound of Formula (II), wherein R¹ is C₁-C₃ alkyl, R² is C₁-C₃ alkyl and R³ is O, or a pharmaceutically acceptable salt thereof.

In another embodiment, the composition administered via the method provided herein comprises an effective amount of a compound of Formula (II), wherein R¹ is C₃-C₁₄ alkyl, R² is C₃-C₁₄ alkyl and R³ is O, or a pharmaceutically acceptable salt thereof.

In another embodiment, the composition administered via the method provided herein comprises an effective amount of a compound of Formula (II), wherein R¹ is C₃-C₉ alkyl, R² is C₃-C₉ alkyl and R³ is O, or a pharmaceutically acceptable salt thereof.

In another embodiment, the composition administered via the method provided herein comprises an effective amount of a compound of Formula (II), wherein R¹ is C₃-C₅ alkyl, R² is C₃-C₅ alkyl and R³ is O, or a pharmaceutically acceptable salt thereof.

In another embodiment, the composition administered via the method provided herein comprises an effective amount of a compound of Formula (II), wherein R¹ is C₅-C₁₃ alkyl, R² is C₅-C₁₃ alkyl and R³ is O, or a pharmaceutically acceptable salt thereof.

In another embodiment, the composition administered via the method provided herein comprises an effective amount of a compound of Formula (II), wherein R¹ is C₅-C₉ alkyl, R² is C₅-C₉ alkyl and R³ is O, or a pharmaceutically acceptable salt thereof.

In another embodiment, the composition administered via the method provided herein comprises an effective amount of a compound of Formula (II), wherein R¹ is C₅-C₇ alkyl, R² is C₅-C₇ alkyl and R³ is O, or a pharmaceutically acceptable salt thereof.

In another embodiment, the composition administered via the method provided herein comprises an effective amount of a compound of Formula (II), wherein R¹ is C₆-C₂₀ alkyl, R² is C₆-C₂₀ alkyl and R³ is O, or a pharmaceutically acceptable salt thereof.

In another embodiment, the composition administered via the method provided herein comprises an effective amount of a compound of Formula (II), wherein R¹ is C₆-C₈ alkyl, R² is C₆-C₈ alkyl and R³ is O, or a pharmaceutically acceptable salt thereof.

In a further embodiment, the composition administered via the method provided herein comprises an effective amount of a compound of Formula (II), wherein R¹ is C₇-C₂₀ alkyl, R² is C₇-C₂₀ alkyl and R³ is O.

In a preferred embodiment, the composition administered via the method provided herein comprises an effective amount of a compound of Formula (II), wherein R¹ is C₈-C₂₀ alkyl, R² is C₈-C₂₀ alkyl and R³ is O.

In a preferred embodiment, the composition administered via the method provided herein comprises an effective amount of a compound of Formula (II), wherein R¹ is C₈-C₁₆ alkyl, R² is C₈-C₁₆ alkyl and R³ is O.

In a further embodiment, the composition administered via the method provided herein comprises an effective amount of a compound of Formula (II), wherein R¹ is C₉-C₂₀ alkyl, R² is C₉-C₂₀ alkyl and R³ is O.

In a preferred embodiment, the composition administered via the method provided herein comprises an effective amount of a compound of Formula (II), wherein R¹ is C₁₀-C₂₀ alkyl, R² is C₁₀-C₂₀ alkyl and R³ is O.

In a preferred embodiment, the composition administered via the method provided herein comprises an effective amount of a compound of Formula (II), wherein R¹ is C₁₀-C₁₆ alkyl, R² is C₁₀-C₁₆ alkyl and R³ is O.

In a preferred embodiment, the composition administered via the method provided herein comprises an effective amount of a compound of Formula (II), wherein R¹ is C₁₂-C₂₀ alkyl, R² is C₁₂-C₂₀ alkyl and R³ is O.

In another embodiment, the composition administered via the method provided herein comprises an effective amount of a compound of Formula (II), wherein R¹ is C₁₂-C₁₈ alkyl, R² is C₁₂-C₁₈ alkyl and R³ is O.

In another embodiment, the composition administered via the method provided herein comprises an effective amount of a compound of Formula (II), wherein R¹ is C₁₂-C₁₆ alkyl, R² is C₁₂-C₁₆ alkyl and R³ is O.

In another embodiment, the composition administered via the method provided herein comprises an effective amount of a compound of Formula (II), wherein R¹ is C₁₄-C₂₀ alkyl, R² is C₁₄-C₂₀ alkyl and R³ is O.

In another embodiment, the composition administered via the method provided herein comprises an effective amount of a compound of Formula (II), wherein R¹ is C₁₄-C₁₆ alkyl, R² is C₁₄-C₁₆ alkyl and R³ is O.

In another embodiment, the composition administered via the method provided herein comprises an effective amount of a compound of Formula (II), wherein R¹ is C₁₈-C₂₀ alkyl, R² is C₁₈-C₂₀ alkyl and R³ is O.

In another embodiment, the composition administered via the method provided herein comprises an effective amount of a compound of Formula (II), wherein R¹ is C₁₆-C₂₀ alkyl, R² is C₁₆-C₂₀ alkyl and R³ is O.

In a preferred embodiment, the composition administered via the method provided herein comprises an effective amount of a compound of Formula (II), wherein R¹ is C₁₂ alkyl, R² is C₁₂ alkyl and R³ is O.

In a further embodiment, the composition administered via the method provided herein comprises an effective amount of a compound of Formula (II), wherein R¹ is C₁₃ alkyl, R² is C₁₃ alkyl and R³ is O.

In a preferred embodiment, the composition administered via the method provided herein comprises an effective amount of a compound of Formula (II), wherein R¹ is C₁₄ alkyl, R² is C₁₄ alkyl and R³ is O.

In a further embodiment, the composition administered via the method provided herein comprises an effective amount of a compound of Formula (II), wherein R¹ is C₁₅ alkyl, R² is C₁₅ alkyl and R³ is O.

In a further embodiment, the composition administered via the method provided herein comprises an effective amount of a compound of Formula (II), wherein R¹ is C₁₆ alkyl, R² is C₁₆ alkyl and R³ is O.

In a further embodiment, the composition administered via the method provided herein comprises an effective amount of a compound of Formula (II), wherein R¹ is C₁₇ alkyl, R² is C₁₇ alkyl and R³ is O.

In a preferred embodiment, the composition administered via the method provided herein comprises an effective amount of a compound of Formula (II), wherein R¹ is C₁₈ alkyl, R² is C₁₈ alkyl and R³ is O.

In a further embodiment, the composition administered via the method provided herein comprises an effective amount of a compound of Formula (II), wherein R¹ is C₁₉ alkyl, R² is C₁₉ alkyl and R³ is O.

In a preferred embodiment, the composition administered via the method provided herein comprises an effective amount of a compound of Formula (II), wherein R¹ is C₂₀ alkyl, R² is C₂₀ alkyl and R³ is O.

In a preferred embodiment, the composition administered via the method provided herein comprises an effective amount of a compound of Formula (II), wherein R¹ is C₁₂ alkyl, R² is C₁₂ alkyl and R³ is O.

In a one embodiment, the composition administered via the method provided herein comprises an effective amount of a compound of Formula (II), wherein R¹ is C₁₆ alkyl, R² is C₁₆ alkyl and R³ is O.

In one embodiment, the disease-modifying antisarcoid compound administered via the methods provided herein is an effective amount of mycophenolate sodium.

In yet another embodiment, the disease-modifying antisarcoid compound administered via the methods provided herein is an effective amount of mycophenolate mofetil.

Granulomatous inflammation in sarcoidosis requires antigen-specific CD4+ T-lymphocytes (Sahoo et al. (2011). Eur. Respir. J. 38, pp. 1145-1150, incorporated by reference herein in its entirety for all purposes). Accordingly, in certain embodiments, the disease-modifying antisarcoid compound administered via the methods provided herein inhibits enzyme dihydro-orotate dehydrogenase, the rate limiting step in de novo synthesis of pyrimidines and progression of the cell cycle in different cell lines, mainly activated T lymphocytes. In one embodiment, the inhibition is inhibition of the de novo synthesis of deoxyuridine monophosphate (dUMP). In the absence of dUMP, p53-mediated apoptosis is triggered in activated, but not resting lymphocytes (Sahoo et al. (2011). Eur. Respir. J. 38, pp. 1145-1150, incorporated by reference herein in its entirety for all purposes). In one embodiment, the dUMP synthesis inhibitor is leflunomide.

Sarcoidosis is a granulomatous disease characterized by enhanced lymphocyte and macrophage activity, and therefore is associated with a number of immune responses. Accordingly, in certain embodiments, the disease-modifying antisarcoid compound administered via the inhalation methods described herein is an immunomodulator that targets one of these responses, for example, the compound targets TNF-α release by alveolar macrophages by inhibiting production of TNF-α, or inhibiting TNF-α binding to one of its receptors. Immunomodulators are diverse compounds, medications, or biologic agents that modify the immune response. They can include cytokines; chemokines; interleukins; synthetic cytosine phosphate-guanosine (CpG) oligodeoxynucleotides; glucans; antibodies; immune effector cells such as lymphocytes, macrophages, dendritic cells, natural killer cells, cytotoxic T lymphocytes; attenuated live bacteria; glucocorticoids; antihistamines; and helminths.

A disease-modifying antisarcoid compound administered via the methods provided herein in one embodiment, is an immunomodulating agent (e.g., an immunosuppressive agent) or a cytotoxic agent. The disease-modifying antisarcoid compound, in one embodiment, is an immunomodulating agent. In a further embodiment, the immunomodulating agent is an immunosuppressive agent. Immunosuppressive agents such as methotrexate, mycophenolate, azathioprine (AZA), cyclosporine, chlorambucil, cyclophosphamide, hydroxychloroquine, indomethacin, pentoxifylline, thalidomide, leflunomide. In another embodiment, the disease-modifying antisarcoid compound is a cytotoxic agent. In a further embodiment, the cytotoxic agent is a natural product, or a derivative thereof. In even a further embodiment, the natural product is colchicine.

In one embodiment, the disease-modifying antisarcoid compound administered via the methods provided herein is a tumor necrosis factor alpha (TNF-α) antagonist. In a further embodiment, the TNF-α antagonist is certolizumab pegol, etanercept, adalimumab, infliximab, azathioprine, golimumab.

Particular compounds that are classified as disease-modifying antisarcoid compounds, and amenable for use in the methods provided herein include, but are not limited to, methotrexate (MTX), azathioprine (AZA), leflunomide, mycophenolate mofetil, mycophenolate acid, mycophenolate sodium, chloroquine, hydroxychloroquine, cyclosporine, chlorambucil, thalidomide, cyclophosphamide, pentoxifylline, a derivative thereof, or a pharmaceutically acceptable salt thereof. In one embodiment, the disease-modifying antisarcoid compound is provided as a prodrug. For example, in one embodiment, the prodrug is an ester, amide or carbamate prodrug. In a further particular embodiment, the prodrug is an alkyl ester. In even a further embodiment, the alkyl ester is an alkyl ester of mycophenolate, or a pharmaceutically acceptable salt thereof.

In one embodiment, the disease-modifying antisarcoid compound administered via the methods provided herein is methotrexate (MTX), a derivative thereof (e.g., a prodrug or conjugate of methotrexate), or a pharmaceutically acceptable salt thereof. Methotrexate is one of the most commonly used corticosteroid-sparing therapies for sarcoidosis, due to its effectiveness, low cost and, at the dosages used to treat sarcoidosis, relatively low risk of side effects compared to other cytotoxic agents. In one embodiment, the MTX is a conjugate, for example, a conjugate described by Abolmaali et al. (Abolmalli et al. (2013). Cancer Chemother. Pharmacol. 71, pp. 1115-1130, incorporated by reference herein in its entirety for all purposes). In one embodiment, the MTX conjugate is a human serum albumin (HSA), dextran, polyethylene glycol, hyaluronic acid, poly(lactic-co-glycolic) acid, gelatin, poly-L-lactic acid, poly-L-lysine, poly(aminodoamine), chitosan or albumin conjugate. In another one embodiment, folic acid supplementation is used when using MTX to reduce toxicity of MTX. In this embodiment, folic acid can be in the same or different composition as MTX. An embodiment of the invention is the use MTX administered via inhalation to a patient to treat pulmonary sarcoidosis. In one embodiment of the invention, it comprises an effective amount of azathioprine (AZA or Imuran®), a derivative thereof or a pharmaceutically acceptable salt thereof, is provided in one of the compositions described herein, for example, to deliver via inhalation to a patient in need of pulmonary sarcoidosis treatment. Azathioprine is a purine analog, and is converted to its active form, 6-mercaptopurine in vivo. In a particular embodiment, a composition comprising an effective amount of azathioprine, a derivative thereof or a pharmaceutically acceptable salt thereof that is administered to a patient with stage (III) or stage (IV) pulmonary sarcoidosis. In a further embodiment, the patient is a candidate for lung transplantation. In yet another embodiment, the patient is administered an AZA composition when there is a contraindication to methotrexate, for example, renal or hepatic function impairment.

In another embodiment of the invention, dosages of methotrexate, a derivative thereof or a pharmaceutically acceptable salt thereof can be adjusted by the prescribing physician. In one embodiment, the dosage is from about 5 mg to about 20 mg weekly, in multiple dosing sessions per week or a single dosing session (e.g., daily, every other day or weekly). In one embodiment, MTX is administered daily, every other day, weekly or monthly. Dosage adjustment may be needed or an alternative corticosteroid-sparing drug may be considered in those with renal insufficiency, e.g., a patient having a serum creatinine>1.5 (gfr<50 ml/min).

Mycophenolic acid ((4E)-6-(4-hydroxy-6-methoxy-7-methyl-3-oxo-1, 3-dihydro-2-benzofuran-5-yl)-4methylhex-4-enoic acid) is an immunosuppressant drug that blocks the biosynthesis of guanine nucleotides via inhibition of inosine 5′-monophosphate dehydrogenase which suppresses the production of proinflammatory cytokines, nitric oxide and LDH in mononuclear phagocytes such macrophages. It has also been shown to inhibit IL-2-dependent T cell proliferation. Accordingly, it can be used in the compositions and methods described herein for the treatment of pulmonary sarcoidosis, for example, by targeting the formation and/or growth of sarcoid granulomas in the lung via inhalation administration.

In one embodiment, mycophenolic acid an effective amount of mycophenolic acid is provided as a pharmaceutically acceptable salt, e.g., mycophenolate sodium, as the free acid, or in prodrug form, for example, as mycophenolate mofetil in one of the compositions described herein.

In another embodiment, an effective amount of a mycophenolic acid is provided as a pharmaceutically acceptable salt, e.g., mycophenolate sodium, as the free acid, or in prodrug form, for example, as mycophenolate mofetil in a composition for delivery to a pulmonary sarcoidosis patient via inhalation.

In one embodiment, an effective amount of mycophenolate sodium is provided in a composition for delivery to a pulmonary sarcoidosis patient via inhalation.

In another embodiment, an effective amount of an immunophilin is provided in a composition, for delivery to a pulmonary sarcoidosis patient via inhalation. In a further embodiment, the immunophilin is the fungal peptide cyclosporine, which has been shown to have T-cell inhibitory effects.

In one embodiment, the disease-modifying antisarcoid compound administered via the methods provided herein is an antimalarial drug. In a further embodiment, the antimalarial drug is chloroquine or hydroxychorloquine, a derivative thereof (e.g., a prodrug) or a pharmaceutically acceptable salt thereof. Both chloroquine and hydroxychorloquine are lysosomotropic basic amines that have been shown to alter the pH in cell vesicles, and have also been shown to inhibit the degradation of proteins by acidic hydrolases within lysomomes, as well as to inhibit the assembly of MHC-peptide complexes and their transport to the cell surface (Moller (2003). Journal of Internal Medicine 253, pp. 31-40, incorporated by reference herein in its entirety for all purposes). Accordingly, and without wishing to be bound by theory, it is thought that these drugs can interfere in the development of pulmonary granulomatous inflammation that is indicative of sarcoidosis.

In one embodiment, the disease-modifying antisarcoid compound administered via the methods provided herein is chloroquine, a derivative thereof (e.g., a prodrug thereof), or a pharmaceutically acceptable salt thereof. Chloroquine is a 4-aminoquinoline drug that has been used previously to treat malaria and some autoimmune disorders such as rheumatoid arthritis and lupus erythematosus. It has also been studied in a limited manner in pulmonary sarcoidosis patients (Judson (2012). Respiratory Medicine 106, pp. 1351-1361, incorporated by reference herein in its entirety for all purposes). Chloroquine derivatives are also amenable for use with the present invention, for example, derivatives that are substituted at the amine groups with an amino acid, peptide or alkyl (straight or branched). Chloroquine dosages amenable for use with the compositions and methods described herein include those described by previously, and can be adjusted according to the knowledge of those of ordinary skill in the art. (Judson (2012). Respiratory Medicine 106, pp. 1351-1361; Balzen et al. (1999). American Journal of Respiratory and Critical Care Medicine 160, pp. 192-197, each incorporated by reference herein in its entirety for all purposes).

In another embodiment, the disease-modifying antisarcoid compound administered via the methods provided herein is hydroxychloroquine, sold under the trade name of Pladquenil®. Hydroxychloroquine differs from chloroquine by the presence of a hydroxyl group at the end of the side chain: The N-ethyl substituent is beta-hydroxylated. Hydroxychloroquine derivatives are also amenable for use with the present invention, for example, derivatives that are substituted at the hydroxyl or amine groups with an amino acid, peptide or alkyl (straight or branched).

In yet another embodiment, the disease-modifying antisarcoid compound administered via the methods provided herein is chlorambucin (Leukeran®, 4-[bis(2-chlorethyl)amino]benzenebutanoic acid).

As provided above, the disease-modifying antisarcoid compound can be a cytotoxic agent. The cytotoxic agent can be a synthetic agent or a natural product, or a derivative thereof.

For example, in one embodiment, the cytotoxic agent provided in one of the compositions described herein is the natural product colchicine ((S)N-(5, 6, 7, 9-tetrahydro-1, 2, 3, 10-tetramethoxy-9-oxobenzo-α-heptalen-7-yl) acetamide).

Another cytotoxic agent amenable for use as a disease-modifying antisarcoid compound is cyclophosphamide ((RS)—N,N-bis(2-chloroethyl)-1,3,2-oxazaphosphinan-2-amine 2-oxide), a nitrogen mustard alkylating agent. Without wishing to be bound by theory, it is thought that cyclophosphamide acts by reducing the number and function of lymphocytes (Jara-Palomares et al. (2011). Updated Guidelines for the Treatment of Pulmonary Sarcoidosis, Sarcoidosis Diagnosis and Management, Prof. Mohammad Hosein Kalantar Motamedi (Ed.), ISBN: 978-953-307-414-6, incorporated by reference herein in its entirety for all purposes). Accordingly, in the methods provided herein, without wishing to be bound by theory, cyclophosphamide is provided in a composition. In one embodiment, the composition is administered to a patient in need thereof to reduce the number of activated T-lymphocytes at the sites of sarcoid granulomas, or the development of sarcoid granulomas in the lung. In another embodiment, the composition is administered and targeted for uptake by a monoculear phagocyte such a macrophage or monocyte at the site of a sarcoid granuloma in the lung.

The composition in one embodiment comprises an effective amount of a TNF-α antagonist as the disease-modifying antisarcoid compound. Two distinct receptors for TNF-α (TNFRs), a 55 kilodalton protein (p55) and a 75 kilodalton protein (p75), exist naturally as monomeric molecules on cell surfaces and in soluble forms. Biological activity of TNF-α is dependent upon binding to either cell surface TNFR. The TNF-α antagonists described herein can inhibit or block endogenous TNF-α from binding one or both of its receptors, either by binding TNF-α directly, or by binding one or both of its receptors. In another embodiment, the TNF-α antagonist inhibits TNF-α production, e.g., production by mononuclear cells, e.g., alveolar macrophages.

In one embodiment, the disease-modifying antisarcoid compound administered via the methods provided herein is a TNF-α antagonist. In a further embodiment, the compound is pentoxifylline or thalidomide. In even a further embodiment, the disease-modifying antisarcoid compound is pentoxifylline, which is a methylxanthine derivative and a nonselective phosphodiesterase inhibitor.

In another embodiment, the disease-modifying antisarcoid compound is thalidomide.

In one embodiment, the TNF-α antagonist is a monoclonal antibody against TNF-α or one of its receptors, a fragment thereof (e.g., a Fab′ fragment), or a TNF-α receptor fusion protein. The monoclonal antibody in one embodiment is a recombinant humanized antibody, or a recombinant humanized Fab′ fragment. In a further embodiment, the TNF-α antagonist is certolizumab pegol, etanercept, adalimumab, infliximab, golimumab.

In one embodiment, the TNF-α antagonist is a monoclonal antibody, e.g., a humanized monoclonal antibody or fragment thereof. In a further embodiment, the TNF-α antagonist is adalimumab (Humirag) or a biosimilar version thereof (e.g., Exemptia marketed by Cadila Healthcare Ltd.).

In another embodiment, the TNF-α antagonist is infliximab (Remicade®), a chimeric monoclonal antibody against TNF-α, a fragment thereof, or a biosimilar version thereof. Infliximab, without wishing to be bound by theory, attenuates the biological activity of TNF-α by binding with high affinity to the soluble and transmembrane forms of TNF-α and inhibits binding of TNF-α with its receptors.

In one embodiment, the TNF-α antagonist is certolizumab pegol, an Fc-free, PEGylated (40 kDa PEG moiety) monoclonal antibody. It has been shown to inhibit signaling in vitro through both the p55 and p75 TNF-α receptors. In one embodiment where certolizumab pegol is administered, it is administered in combination with MTX either in the same or a different composition.

In yet another embodiment, the TNF-α antagonist is golimumab, a human monoclonal antibody which targets TNF-α. In another embodiment, the TNF-α antagonist is an antigen biding portion of golimumab, or a biosimilar version of golimumab (or a fragment thereof).

A composition in one embodiment comprises an effective amount of etanercept (Enbrel®). Etanercept is a fusion protein produced by recombinant DNA technology, and fuses the TNF receptor to the constant end of the IgG1 antibody. Etanercept is a dimeric soluble form of the p75 TNF receptor that can bind TNF-α molecules.

As provided in further detail below, a composition provided herein, in some embodiments, comprise a disease-modifying antisarcoid compound complexed to or encapsulated by a lipid component.

Pulmonary sarcoidosis has been classified in different stages according to chest radiography, and the methods provided herein can be used to treat a patient at any stage of the disease. Stage (O): no intrathoracic involvement; stage (I): bilateral hilar lymphadenopathy; stage (II): bilateral hilar lymphadenopathy and reticulonodular infiltrates; stage (III): pulmonary infiltrates with fibrosis; and stage (IV): end-stage lung disease with pulmonary fibrosis and honeycombing. The present invention is amenable for use for the treatment of a subject with stage (O), stage (I), stage (II), stage (III) and/or stage (IV) pulmonary sarcoidosis.

Without wishing to be bound by theory, it is thought that the present invention provides more direct and effective pulmonary sarcoidosis treatment methods by delivering a disease-modifying antisarcoid compound directly to the sites of sarcoid granulomas in the lung and to the sites of granuloma formation. Additionally, delivery of a disease-modifying antisarcoid compound directly to the site of the sarcoidosis infection without wishing to be bound by theory allows for pulmonary fibrosis to be attenuated and/or prevented in treated patients.

In one embodiment, the pulmonary sarcoidosis treatable by the methods, compositions and kits provided herein is necrotising sarcoid granulomatosis (NSG), which is characterized by sarcoid like granuloma formation, vasculitis and variable degrees of necrosis.

In another embodiment, the patient has been diagnosed with alveolar sarcoidosis. Alveolar sarcoidosis, without wishing to be bound by a theory, is thought to result from aggregation of large numbers of interstitial granulomas rather than representing a true alveolar process. In patients with alveolar sarcoidosis, there can be large areas of pulmonary opacification ranging in diameter from 1 to 4 cm. These can be rounded or elongated in shape, have irregular edges and blurred margins with or without air bronchograms. They are typically found either along the bronchovascular bundles or in the lung periphery adjacent to the pleural surface. Small nodules can be often visible around these large opacities, which is often termed the galaxy sign. Another pattern of alveolar sarcoidosis is an appearance termed “fairy ring” which refers to circumferentially organized opacities.

In yet another embodiment, the subject has been diagnosed with cavitatory pulmonary sarcoidosis. Cavitatory pulmonary sarcoidosis is usually reported in those with severe and active disease and its reported prevalence is around 2% of all pulmonary sarcoidosis (Hours et al. Medicine (Baltimore) 87, pp. 142-151, incorporated by reference herein in its entirety for all purposes).

In one embodiment, a composition of the present invention is administered to a patient via inhalation, wherein the patient has pulmonary sarcoidosis resistant to steroid treatment. In another embodiment, the patient was non-responsive to previous sarcoidosis treatment, or experienced adverse effects from a previous sarcoidosis treatment.

In one embodiment, the patient has cutaneous sarcoidosis in addition to pulmonary sarcoidosis.

As provided above, in one embodiment, the subject is a human. The human subject can be a child (i.e., ≦eighteen years old) or adult (i.e., ≧eighteen years old).

The majority of reported childhood sarcoidosis cases have occurred in patients aged 13-15 years old (Shetty and Gedalia (2008). Pediatric Rheumatology 6:16 DOI: 10.1186/1546-0096-6-16, incorporated by reference herein in its entirety for all purposes). However, in one study of childhood sarcoidosis associated with joint involvement, the mean age at onset was 10.6 years (range, 0.1-16 years) (Lindsley and Petty (2000). Curr. Reheumatol. Rep. 2, pp. 343-348, incorporated by reference herein in its entirety for all purposes). Importantly, the methods provided herein are not limited to a particular age of a subject. For example, in one embodiment, the methods provided herein are amenable for use with teen-aged patients, e.g., from about 13 years old to about 18 years old. In another embodiment, the subject is from about 5 years old to about 13 years old, for example from about 5 years old to about 12 years old, or about 10 years old.

In one embodiment, the subject treated with the methods, compositions and kits provided herein is from about 25 years old to about 40 years old.

In another embodiment, the subject is from about 1 month to about 6 months old, from about 6 months to about 12 months old, from about 1 year old to about 5 years old from about, from about 5 to about 10 years old, from about 10 to about 15 years old, from about 15 to 20 years old, from about 20 to 25 years old, 25 to about 30 years old at the onset of treatment, from about 30 to about 35 years old, from about 35 to about 40 years old at the onset of treatment, from about 40 to about 45 years old, from about 45 to about 50 years old at the onset of treatment, from about 50 to about 55 years old, from about 55 to about 60 years old at the onset of treatment, from about 60 to about 65 years old, from about 65 to about 70 years old at the onset of treatment, from about 70 to about 75 years old at the onset of treatment, from about 75 to about 80 years old, from about 80 to about 85 years old, from about 85 to about 90 years old, from about 90 to 95 years old, or from about 95 to 100 years old.

In one embodiment, the pulmonary sarcoidosis patient treated with the methods provided herein has a pre-existing, simultaneous or subsequent malignancy. In a further embodiment, the malignancy comprises a lymphoma, a leukemia, lung cancer, uterine cancer, thyroid cancer, laryngeal cancer, pharyngeal cancer, skin cancer, liver cancer, breast cancer, prostate cancer and colon cancer.

Administration of a composition comprising an effective amount of one or more of the compounds provided herein occurs through pulmonary delivery to the lungs of a patient, for example via a nebulizer, soft mist inhaler, dry powder inhaler (DPI), or a metered dose inhaler (MDI). In some embodiments, a composition comprising an effective amount of one of the compounds provided herein is administered via a nebulizer to a patient in need of pulmonary sarcoidosis treatment. In some embodiments a compound described herein is suspended in a propellant and delivered to a patient via an MDI.

The methods provided herein also include the administration of an effective amount of a metabolite of a disease-modifying antisarcoid compound, for example, a metabolite of one of the compounds described herein. Metabolites from chemical compounds, whether a natural product or pharmaceutical metabolite, can be administered to a patient in need of sarcoidosis treatment, and can be present in one or more of the compositions described herein.

The compounds of the present disclosure can also be present in a composition of a prodrug. In one embodiment, an ester prodrug, for example, pharmaceutically acceptable ester prodrug is present in one of the compositions provided herein and delivered to a patient in need of sarcoidosis treatment via inhalation. For example, a carboxylic acid function group in a compound can be converted to its corresponding ester, e.g., a straight chain or branched alkyl ester. In another embodiment, an alcohol or hydroxyl functional group in a disease-modifying antisarcoid compound is converted to an ester, for example, a straight chain or branched chain alkyl ester, according to the a method known to one of ordinary skill in the art.

In one embodiment, upon in vivo administration, a prodrug of the disease-modifying antisarcoid compound is chemically converted to the biologically, pharmaceutically or therapeutically more active form. Without wishing to be bound by theory, administering a prodrug provides one or more advantages over the administration of the corresponding active form. For example, in certain instances, a prodrug is more bioavailable than the corresponding active form. In one embodiment, a prodrug has improved solubility compared to the corresponding active form. In another embodiment, the prodrug is less water soluble than the corresponding active form.

In one embodiment, the prodrug of the disease-modifying antisarcoid compound comprises a short peptide (e.g., from about 2 to about 9 amino acids) or a single amino acid bound to an acid group of the compound. In such embodiments, the peptide or amino acid is cleaved upon administration to form the corresponding active form.

In one embodiment, a patient is administered a composition comprising an effective amount of a disease-modifying antisarcoid compound via inhalation for the treatment of pulmonary sarcoidosis once daily, twice daily or three times daily. In another embodiment, administration of the composition occurs every other day or once per week.

In one embodiment, the effective amount of the compound in the composition is from about 0.10 mg to about 0.75 mg. In another embodiment, the effective amount is from about 0.20 mg to about 0.60 mg. In another embodiment, the effective amount is from about 0.25 mg to about 0.50 mg. In a further embodiment, the effective amount of the compound is about 0.50 mg.

The dosage regimen utilizing the compounds is selected in accordance with a variety of factors including type, species, age, weight, sex and medical condition of the patient; the severity of the condition to be treated; the renal and hepatic function of the patient; and the particular compound or salt thereof employed. An ordinarily skilled physician or veterinarian can determine and prescribe the effective amount of the drug required to prevent, counter or arrest the progress of the condition.

As provided above, the disease-modifying antisarcoid compounds, derivatives thereof, or pharmaceutically acceptable salts thereof, of the present invention can be delivered to a patient in need thereof via a pulmonary route. With respect to the pulmonary route, the disease-modifying antisarcoid compound, derivative thereof, or a pharmaceutically acceptable salt thereof, of the present invention may be used in any dosage dispensing device adapted for such administration. The inhalation delivery device can be a nebulizer, dry powder inhaler, or a metered dose inhaler (MDI), or any other suitable inhalation delivery device known to one of ordinary skill in the art. The device can contain and be used to deliver a single dose of the disease-modifying antisarcoid compound or composition or the device can contain and be used to deliver multi-doses of the disease-modifying antisarcoid compound or composition of the present invention. The device, in one embodiment, is constructed to ascertain optimum metering accuracy and compatibility of its constructive elements, such as container, valve and actuator with the formulation and could be based on a mechanical pump system. For example, inhalation delivery devices include a jet nebulizer, electronic nebulizer, a soft mist inhaler, and a capsule-based dry powder inhaler. In one embodiment, where compound delivery is via a nebulizer, the compound is provided to the patient as a composition comprising a lipid component.

In one embodiment, a metered dose inhalator (MDI) is employed as the inhalation delivery device for the compositions of the present invention. In a further embodiment, the disease-modifying antisarcoid compound or composition of the invention is suspended in a propellant (e.g., hydrofluorocarbon) prior to loading into the MDI. The basic structure of the MDI comprises a metering valve, an actuator and a container. A propellant is used to discharge the formulation from the device. The composition may consist of particles of a defined size suspended in the pressurized propellant(s) liquid, or the composition can be in a solution or suspension of pressurized liquid propellant(s). The propellants used are primarily atmospheric friendly hydrofluoroalkanes (HFAs) such as 134a and 227. The device of the inhalation system may deliver a single dose via, e.g., a blister pack, or it may be multi dose in design. The pressurized metered dose inhalator of the inhalation system can be breath actuated to deliver an accurate dose of the lipid-containing formulation. To ensure accuracy of dosing, the delivery of the formulation may be programmed via a microprocessor to occur at a certain point in the inhalation cycle. The MDI may be portable and hand held.

In one embodiment, a compound of the present invention, or a composition comprising the same, is administered via a metered dose inhaler (MDI) to a patient in need of sarcoidosis treatment. The patient, in one embodiment, is administered the disease-modifying antisarcoid compound or composition of the invention once daily or twice daily. In one embodiment, the administration is with food. In one embodiment, each administration comprises 1 to 5 doses (puffs) from an MDI, for example 1 dose (1 puff), 2 dose (2 puffs), 3 doses (3 puffs), 4 doses (4 puffs) or 5 doses (5 puffs). The MDI, in one embodiment, is small and transportable by the patient.

In another embodiment, the disease-modifying antisarcoid compound is administered via a nebulizer to a patient in need of sarcoidosis treatment. The administration occurs, in one embodiment, once daily or twice daily, or once weekly, twice weekly or three times weekly.

In one embodiment, a composition or compound of the present invention is administered to a patient in need thereof via a dry powder inhaler (DPI) to a patient in need of pulmonary sarcoidosis treatment. The patient, in one embodiment, is administered the disease-modifying antisarcoid compound or composition of the invention once daily or twice daily. In one embodiment, the administration is with food. In one embodiment, each administration comprises 1 to 5 doses (puffs) from a DPI, for example 1 dose (1 puff), 2 dose (2 puffs), 3 doses (3 puffs), 4 doses (4 puffs) or 5 doses (5 puffs). The DPI, in one embodiment, is small and transportable by the patient.

The compositions of the present invention may be used in any dosage dispensing device adapted for pulmonary administration. Accordingly, in one aspect, the present invention provides systems comprising one or more of the compositions described herein and an inhalation delivery device. The device, in one embodiment, is constructed to ascertain optimum metering accuracy and compatibility of its constructive elements, such as container, valve and actuator with the composition and could be based on a mechanical pump system, e.g., that of a metered-dose nebulizer, dry powder inhaler, metered dose inhaler (MDI), soft mist inhaler, or a nebulizer. For example, pulmonary delivery devices include a jet nebulizer, electronic nebulizer, a soft mist inhaler, and a capsule-based dry powder inhaler, all of which are amenable for use with the compositions of the present invention.

The composition, in one embodiment, is administered via a nebulizer, which provides an aerosol mist of the composition for delivery to the lungs of a subject in need of treatment. A nebulizer type inhalation delivery device can contain the compositions of the present invention as an aqueous solution or a suspension. In generating the nebulized spray of the compositions for inhalation, the nebulizer type delivery device may be driven ultrasonically, by compressed air, by other gases, electronically or mechanically. The ultrasonic nebulizer device usually works by imposing a rapidly oscillating waveform onto the liquid film of the composition via an electrochemical vibrating surface. At a given amplitude the waveform becomes unstable, whereby it disintegrates the liquids film, and it produces small droplets of the composition. The nebulizer device driven by air or other gases operates on the basis that a high pressure gas stream produces a local pressure drop that draws the liquid composition into the stream of gases via capillary action. This fine liquid stream is then disintegrated by shear forces.

A nebulizer type inhalation delivery device can contain the compositions of the present invention as a solution, usually aqueous, or a suspension. For example, the composition can be suspended in saline and loaded into the inhalation delivery device. In generating the nebulized spray of the compositions for inhalation, the nebulizer delivery device may be driven ultrasonically, by compressed air, by other gases, electronically or mechanically (e.g., vibrating mesh or aperture plate). Vibrating mesh nebulizers generate fine particle, low velocity aerosol, and nebulize therapeutic solutions and suspensions at a faster rate than conventional jet or ultrasonic nebulizers. Accordingly, the duration of treatment can be shortened with a vibrating mesh nebulizer, as compared to a jet or ultrasonic nebulizer. Vibrating mesh nebulizers amenable for use with the methods described herein include the Philips Respironics I-Neb®, the Omron MicroAir, the Nektar Aeroneb®, and the PARI eFlow®. Other devices that can be used with the compositions described herein include jet nebulizers (e.g., PARI LC Star, AKITA), soft mist inhalers, and capsule-based dry powder inhalers (e.g., PH&T Turbospin).

The nebulizer may be portable and hand held in design, and may be equipped with a self-contained electrical unit. The nebulizer device may comprise a nozzle that has two coincident outlet channels of defined aperture size through which the liquid composition can be accelerated. This results in impaction of the two streams and atomization of the composition. The nebulizer may use a mechanical actuator to force the liquid composition through a multiorifice nozzle of defined aperture size(s) to produce an aerosol of the composition for inhalation. In the design of single dose nebulizers, blister packs containing single doses of the composition may be employed.

The device can contain, and be used to deliver, a single dose of the compositions of the invention, or the device can contain, and be used to deliver, multi-doses of the compositions of the invention.

In the present invention the nebulizer may be employed to ensure the sizing of particles is optimal for positioning of the particle within, for example, the pulmonary membrane.

A metered dose inhalator (MDI) may be employed as the inhalation delivery device for the compositions of the present invention. This device is pressurized (pMDI) and its basic structure comprises a metering valve, an actuator and a container. A propellant is used to discharge the composition from the device. Suitable propellants, e.g., for MDI delivery, may be selected among such gases as fluorocarbons, chlorofluorocarbons (CFCs), hydrocarbons, hydrofluorocarbons, hydrofluoroalkane propellants (e.g., HFA-134a and HFA-227), nitrogen and dinitrogen oxide or mixtures thereof.

In one embodiment, a propellant is present in a composition intended for MDI delivery, and is selected from a fluorocarbon, chlorofluorocarbon (CFC), hydrocarbons, hydrofluoroalkane propellants (e.g., HFA-134a and HFA-227), nitrogen and dinitrogen oxide or mixtures thereof. In embodiments of the present invention, the propellant is CFC-12 or an ozone-friendly, non-CFC propellant, such as 1,1,1,2-tetrafluoroethane (HFC 134a), 1,1,1,2,3,3,3-heptafluoropropane (HFA-227), HFA-152 (difluoroethane and isobutene), trans-1,3,3,3,-tetrafluoropro-1-ene (HFO 1234ze) and 2,3,3,3,-tetrafluoroprop-1-ene (HFO 1234yf), or combinations thereof.

The composition may consist of particles of a defined size suspended in the pressurized propellant(s) liquid, or the composition can be in a solution or suspension of pressurized liquid propellant(s). The propellants used are primarily atmospheric friendly hydroflourocarbons (HFCs) such as 134a and 227. The inhalation delivery device, in one embodiment, delivers a single dose via, e.g., a blister pack, or it may be multi dose in design. The pressurized metered dose inhalator of the inhalation system can be breath actuated to deliver an accurate dose of the composition. To insure accuracy of dosing, the delivery of the composition may be programmed via a microprocessor to occur at a certain point in the inhalation cycle. The MDI may be portable and hand held.

For MDI delivery, in one embodiment, the disease-modifying antisarcoid compound is reduced in particle size prior to formulating in a composition. Particle size reduction can be achieved by milling, spray drying or using supercritical fluids. Milling can include cryo milling, ball milling, fluid-energy milling and cryogenic continuous bead milling. Ball mills and fluid-energy mills (such as jet mills) are the primary modes of milling powders to achieve particles with diameters of 1 to 5 μm. Ball mills use balls that grind the drug as the balls tumble inside the mill. Jet milling reduces particle size of coarse powders by high velocity particle-particle collisions. Alternatively, spray drying may be used to reduce particle size. Spray drying converts a solution or liquid dispersion (also known as “feed”) to dried particulates by the process of atomizing a spray of the liquid containing the drug followed by quickly drying the droplets, which yields solid particles. Compared to milling, spray drying often produces relatively spherical, amorphous particles. Finally, supercritical fluids may also be utilized to manufacture particles for inhalation. A supercritical fluid is any substance at a temperature and pressure above its critical point, the point where both the liquid and gas phases have the same density. The drug is dissolved in the supercritical fluid, at high pressure and temperature, followed by decrease in pressure and/or temperature which yields a reduction in the density of the solution, thereby decreasing the solvation power of the supercritical fluid, leading to precipitation of the drug. Supercritical fluids can be used in multiple ways to micronize drug particles. They may be used to micronize drug material through rapid expansion of supercritical solutions, using supercritical fluid as an antisolvent and precipitation of particles from gas saturated solutions. Particle size reduction can also be done by an emulsion template process (Dugas et al., 2013, International Journal of Pharmaceutics 441: 19-29, incorporated by reference herein in its entirety for all purposes).

In one embodiment, an effective amount of a disease-modifying antisarcoid compound, a derivative thereof, or a pharmaceutically acceptable salt thereof, is reduced in particle size. In another embodiment, the particle size is reduced by milling, spray drying, using supercritical fluids, and/or by an emulsion template process. In a further embodiment, the compound is passed through a sieve. In yet another embodiment, the sieve size is about 5 μm.

Yet another aspect of the invention relates to the compositions described above in aerosolized form. Upon nebulization or aerosolization, the aerosolized composition is in the form of aerosolized particles. The aerosolized composition can be characterized by the particle size of the aerosol, for example, by measuring the “mass median aerodynamic diameter” or “fine particle fraction” associated with the aerosolized composition. “Mass median aerodynamic diameter” or “MMAD” is normalized regarding the aerodynamic separation of aqua aerosol droplets and is determined by impactor measurements, e.g., the Anderson Cascade Impactor (ACI) or the Next Generation Impactor (NGI). The gas flow rate, in one embodiment, is 28 Liter per minute for the ACI and 15 liter per minute for the NGI.

“Geometric standard deviation” or “GSD” is a measure of the spread of an aerodynamic particle size distribution. Low GSDs characterize a narrow droplet size distribution (homogeneously sized droplets), which is advantageous for targeting aerosol to the respiratory system. The average droplet size of the nebulized composition provided herein, in one embodiment is less than 5 μm or about 1 μm to about 5 μm, and has a GSD in a range of 1.0 to 2.2, or about 1.0 to about 2.2, or 1.5 to 2.2, or about 1.5 to about 2.2.

“Fine particle fraction” or “FPF,” as used herein, refers to the fraction of the aerosol having a particle size less than 5 μm in diameter, as measured by cascade impaction. FPF is usually expressed as a percentage

In the present invention the nebulizer may be employed to ensure the sizing of particles is optimal for positioning of the particle within, for example, the pulmonary membrane.

In one embodiment, the mass median aerodynamic diameter (MMAD) of the aerosol particles is about 1 μm to about 5 μm, or about 1 μm to about 4 μm, or about 1 μm to about 3 μm, or about 2 μm to about 3 μm, or about 1 μm to about 2 μm, as measured by cascade impaction, for example, by the ACI or NGT.

In another embodiment, the MMAD of the aerosol particles is about 5 μm or less, about 4 μm or less, about 3 μm or less, about 2 μm or less, or about 1 μm or less, as measured by cascade impaction, for example, by the ACI or NGT.

“Geometric standard deviation” or “GSD” is a measure of the spread of an aerodynamic particle size distribution. Low GSDs characterize a narrow droplet size distribution (homogeneously sized droplets), which is advantageous for targeting aerosol to the respiratory system. The average droplet size of the aerosolized composition provided herein, in one embodiment is less than 5 μm or about 1 μm to about 5 μm, and has a GSD in a range of from about 1.0 to about 2.2, or from about 1.5 to about 2.2, as measured by the ACI or NGT.

“Respirable mass” or “RM”, as used herein, is usually expressed as μg/shot and is the total amount of emitted drug product that exits the metered dose inhaler upon actuation.

In one embodiment, the respirable mass of the aerosol particles is about 1 μg/shot to about 100 μg/shot, or about 1 μg/shot to about 50 μg/shot, or about 1 μg/shot to about 40 μg/shot, or about 1 μg/shot to about 30 μg/shot, or about 3 μg/shot to about 80 μg/shot, or about 3 μg/shot to about 70 μg/shot, or about 3 μg/shot to about 60 μg/shot, about 3 μg/shot to about 50 μg/shot, about 3 μg/shot to about 40 μg/shot, about 3 μg/shot to about 30 μg/shot, as measured by the ACI or NGT.

“Fine particle fraction” or “FPF”, as used herein, refers to the fraction of the aerosol having a particle size less than 5 μm in diameter, as measured by cascade impaction. FPF is usually expressed as a percentage.

In one embodiment, the fine particle fraction (FPF) of the aerosol particles is greater is greater than or equal to about 40%, is greater than or equal to about 50%, is greater than or equal to about 60%, is greater than or equal to about 70%, is greater than or equal to about 80%, greater than or equal to about 85%, greater than or equal to about 90%, or greater than or equal to about 95%, as measured by the ACI or NGT.

In another embodiment, the FPF of the aerosol particles is about 40% to about 99%, is about 50% to about 99%, is about 60% to about 99%, is about 70% to about 99%, is about 75% to about 99%, is about 80% to about 99%, is about 80% to about 95%, is about 80% to about 90%, or is about 85% to about 90%, or is about 85% to about 95%, as measured by the ACI or NGT.

“Percent throat deposition” or “PTD” is the amount of drug deposited on the throat of the cascade impactor and is expressed as a percentage.

In one embodiment, the percent throat deposition is less than or equal to about 60%, less than or equal to about 50%, less than or equal to about 40%, less than or equal to about 30%, less than or equal to about 25%, as measured by the ACI or NGT.

In one embodiment, a dry powder inhaler (DPI) is employed as the inhalation delivery device for the compositions of the present invention. In one embodiment, the DPI generates particles having an MMAD of from about 1 μm to about 10 μm, or about 1 μm to about 9 μm, or about 1 μm to about 8 μm, or about 1 μm to about 7 μm, or about 1 μm to about 6 μm, or about 1 μm to about 5 μm, or about 1 μm to about 4 μm, or about 1 μm to about 3 μm, or about 1 μm to about 2 μm in diameter, as measured by the NGT or ACI. In another embodiment, the DPI generates a particles having an MMAD of from about 1 μm to about 10 μm, or about 2 μm to about 10 μm, or about 3 μm to about 10 μm, or about 4 μm to about 10 μm, or about 5 μm to about 10 μm, or about 6 μm to about 10 μm, or about 7 μm to about 10 μm, or about 8 μm to about 10 μm, or about 9 μm to about 10 μm, as measured by the NGT or ACI.

In one embodiment, the MMAD of the particles generated by the DPI is about 10 μm or less, about 9 μm or less, about 8 μm or less, about 7 μm or less, about 6 μm or less, about 5 μm or less, about 4 μm or less, about 3 μm or less, about 2 μm or less, or about 1 μm or less, as measured by the NGI or ACI.

In one embodiment, the MMAD of the particles generated by the DPI is less than about 9.9 μm, less than about 9.5 μm, less than about 9.3 μm, less than about 9.2 μm, less than about 9.1 μm, less than about 9.0 μm, less than about 8.5 μm, less than about 8.3 μm, less than about 8.2 μm, less than about 8.1 μm, less than about 8.0 μm, less than about 7.5 μm, less than about 7.3 μm, less than about 7.2 μm, less than about 7.1 μm, less than about 7.0 μm, less than about 6.5 μm, less than about 6.3 μm, less than about 6.2 μm, less than about 6.1 μm, less than about 6.0 μm, less than about 5.5 μm, less than about 5.3 μm, less than about 5.2 μm, less than about 5.1 μm, less than about 5.0 μm, less than about 4.5 μm, less than about 4.3 μm, less than about 4.2 μm, less than about 4.1 μm, less than about 4.0 μm or less than about 3.5 μm, as measured by the NGI or ACI.

In one embodiment, the MMAD of the particles generated by the DPI is from about 1.0 μm to about 10.0 μm, from about 2.0 μm to about 9.5 μm, from about 2.5 μm to about 9.0 μm, from about 3.0 μm to about 9.0 μm, from about 3.5 μm to about 8.5 μm or from about 4.0 μm to about 8.0 μm.

In one embodiment, the FPF of the disease-modifying antisarcoid particulate composition generated by the DPI is greater than or equal to about 40%, greater than or equal to about 50%, greater than or equal to about 60%, or greater than or equal to about 70%, as measured by the ACI or NGI. In another embodiment, the FPF of the aerosolized composition is about 80% to about 99%, about 80% to about 95%, about 80% to about 90%, or about 85% to about 90%, or about 85% to about 95%, as measured by the NGI or ACI.

Symptoms of pulmonary sarcoidosis include dry cough, fatigue, shortness of breath, weight loss, tender reddish bumps or patches on the skin, inflammation of the eyes, swollen and painful joints, enlarged and tender lymph glands in the neck, armpits, and groin, enlarged lymph glands in the chest and around the lungs, hoarse voice, pain in the hands, feet, or other bony areas due to the formation of cysts (an abnormal sac-like growth) in bones, kidney stone formation, enlarged liver, development of abnormal or missed heart beats (arrhythmias), inflammation of the covering of the heart (pericarditis), or heart failure, nervous system effects, including hearing loss, meningitis, seizures, or psychiatric disorders (for example, dementia, depression, psychosis).

In one aspect of the invention, inhalation administration of one of the compositions provided herein to a patient in need of pulmonary sarcoidosis treatment results in a decreased number of pulmonary sarcoidosis symptoms experienced by the patient, or a decreased severity of one or more symptoms experienced by the patient, as compared to the number of symptoms or severity of the one or more symptoms experienced by the patient prior to administration of the composition.

Lofgren's syndrome is a classic set of signs and symptoms that is typical in some people who have sarcoidosis. Lofgren's syndrome may cause fever, enlarged lymph nodes, arthritis (usually in the ankles), and/or erythema nodosum, a rash of red or reddish-purple bumps on ankles and shins. The present invention, in one embodiment, serves to decrease one or more symptoms of Lofgren's syndrome in a patient via inhalation of one of the compositions provided herein, as compared to the number or severity of the one or more symptoms prior to administration of the composition.

In another embodiment, the inhalation administration of one of the compositions provided herein results in a decreased number of sarcoidosis symptoms experienced by the patient, or a decreased severity of one or more symptoms experienced by the patient, as compared to the number of symptoms or severity of the one or more symptoms experienced by the patient when administered the same antisarcoid compound present in the composition (or a derivative or pharmaceutically acceptable salt thereof) via a non-inhalation route of administration. In a further embodiment, the non-inhalation route of administration is subcutaneous, intravenous or oral.

In another embodiment, the administration of the effective amount of one of the compositions provided herein results in a decreased number of sarcoidosis symptoms experienced by the patient, or a decreased severity of the one or more symptoms experienced by the patient, as compared to the number of symptoms or severity of the one or more symptoms experienced by the patient when administered a corticosteroid compound, a derivative thereof, or pharmaceutically acceptable salt thereof, via oral or inhaled administration. In one embodiment, the corticosteroid compound is prednisone, prednisolone, flunisolide, fluticasone furoate, fluticasone propionate, triamcinolone acetonide, beclomethasone dipropionate and/or budesonide.

In one embodiment, the one or more symptoms is dry cough, fatigue, shortness of breath, weight loss, tender reddish bumps or patches on the skin, inflammation of the eyes, swollen and painful joints, enlarged and tender lymph glands in the neck, armpits, and groin, enlarged lymph glands in the chest and around the lungs, hoarse voice, pain in the hands, feet, or other bony areas due to the formation of cysts (an abnormal sac-like growth) in bones, kidney stone formation, enlarged liver, development of abnormal or missed heart beats (arrhythmias), inflammation of the covering of the heart (pericarditis), or heart failure, nervous system effects, including hearing loss, meningitis, seizures, or psychiatric disorders (for example, dementia, depression, psychosis).

Fatigue is very often manifested in sarcoidosis patients. A 10-item Fatigue Assessment Scale (FAS) has been developed to measure fatigue in sarcoidosis patients and to assess progress in combating fatigue during treatment (Michielson et al. 2004, Eur. J. Psychological Assessment 20(1): 39-48, incorporated by reference in its entirety herein for all purposes). The scale indicates both physical and psychological fatigue. Each item has a five-point rating scale and FAS scores range from 10 to 50. FAS scores<22 indicate nonfatigued persons, scores of 22-34 indicate fatigued persons and scores of ≧35 indicate extremely fatigued persons. The psychometric properties of the FAS are also good in sarcoidosis.

In one embodiment, administration of one of the compositions provided herein results in decreased severity of fatigue. In another embodiment, the decreased severity of fatigue is measured by the Fatigue Assessment Scale (FAS). In one embodiment the severity of fatigue decreases at least about 1 point, by at least about 2 points, by at least about 3 points, by at least about 4 points, by at least about 5 points, by at least about 6 points, by at least about 7 points, by at least about 8 points, by at least about 9 points, by at least about 10 points, by at least about 11 points, by at least about 12 points, by at least about 13 points, by at least about 14 points, by at least about 15 points, by at least about 16 points, by at least about 17 points, by at least about 18 points, by at least about 19 points, by at least about 20 points, by at least about 21 points, by at least about 22 points, by at least about 23 points, by at least about 24 points, by at least about 25 points, by at least about 26 points, by at least about 27 points, by at least about 28 points, by at least about 29 points, by at least about 30 points, by at least about 31 points, by at least about 32 points, by at least about 33 points, by at least about 34 points, by at least about 35 points, by at least about 36 points, by at least about 37 points, by at least about 38 points, by at least about 39 points or by at least about 40 points, as measured by the FAS.

In one embodiment, administration of a composition of the present invention for the treatment of pulmonary sarcoidosis via inhalation results in reduced inflammation in the patient, as compared to the inflammation experienced by the patient prior to administration of the composition.

In one embodiment, administration of one of the compositions provided herein to a patient in need of pulmonary sarcoidosis treatment via inhalation results in reduced inflammation experienced by the patient, as compared to the inflammation experienced by the patient when administered the same disease-modifying antisarcoid compound, a derivative thereof, or pharmaceutically acceptable salt thereof, via a different route of administration, e.g., an oral, subcutaneous or intravenous route of administration.

In one embodiment, administration of one of the compositions provided herein to a patient in need of pulmonary sarcoidosis treatment via inhalation results in reduced inflammation experienced by the patient, as compared to the inflammation experienced by the patient when administered a corticosteroid compound, a derivative thereof, or pharmaceutically acceptable salt thereof, via oral or inhaled administration. In a further embodiment, the corticosteroid compound is prednisone, prednisolone, flunisolide, fluticasone furoate, fluticasone propionate, triamcinolone acetonide, beclomethasone dipropionate and/or budesonide.

Patients can be evaluated by chest radiographs (X-rays), CT scan of chest, positron emission tomography scan, CT-guided biopsy, mediastinoscopy, open lung biopsy, bronchoscopy with biopsy, endobronchial ultrasound, and endoscopic ultrasound with fine needle aspiration of mediastinal lymph nodes to determine whether they are in need of treatment and whether treatment is effective.

Pulmonary function tests are used routinely in evaluation and follow-up of pulmonary sarcoidosis patients. “Forced vital capacity” (FVC) denotes the volume of gas which is exhaled during a forced expiration starting from a position of full inspiration and ending at complete expiration and is one measure of treatment efficacy. “Forced expiratory volume in one second” (FEV₁) is another measure of treatment efficacy and is the volume of gas exhaled in a specified time (typically 1 second) from the start of the forced vital capacity maneuver (Quanjer et al. (1993). Eur. Respir. J. 6, Suppl. 16, pp. 5-40, incorporated by reference herein in its entirety for all purposes). FVC and FEV₁ are measured with a pneumotachograph and are usually expressed as a percentage predicted (FVC %, FEV₁%).

The diffusing capacity of the lung for carbon monoxide (DLCO) is the extent to which oxygen passes from the air sacs of the lungs into the blood. The DLCO test involves measuring the partial pressure difference between inspired and expired carbon monoxide. It relies on the strong affinity and large absorption capacity of erythrocytes for carbon monoxide and thus demonstrates gas uptake by the capillaries that are less dependent on cardiac output. FVC %, FEV₁% and DLCO are decreased in sarcoidosis patients. In one embodiment, an increase in one or more of these measurements denotes an effective treatment.

In one embodiment, administration of one of the compositions provided herein via inhalation results in improved percentage predicted forced vital capacity (FVC %), percentage predicted forced expiratory volume in one second (FEV₁%), and/or chest radiograph of the patient, as compared to a FVC %, FEV₁% and/or a chest radiograph of the patient prior to treatment, or as compared to a FVC %, FEV₁% and/or a chest radiograph improvement experienced by a pulmonary sarcoidosis patient undergoing corticosteroid treatment.

In one embodiment, the FVC % of a patient administered a composition of the present invention via inhalation is greater by about 1%, greater by about 2%, greater by about 3%, greater by about 4%, greater by about 5%, greater by about 6%, greater by about 7%, greater by about 8%, greater by about 9%, greater by about 10%, greater by about 11%, greater by about 12%, greater by about 13%, greater by about 14%, greater by about 15%, greater by about 16%, greater by about 17%, greater by about 18%, greater by about 19%, greater by about 20%, greater by about 25%, greater by about 30%, greater by about 35%, greater by about 40%, greater by about 45%, greater by about 50%, greater by about 55%, greater by about 60%, greater by about 65%, greater by about 70%, greater by about 75%, greater by about 80%, greater by about 85%, greater by about 90%, and all values in between compared to a FVC % of the patient prior to treatment.

In another embodiment, the FEV₁% of a patient administered a disease-modifying antisarcoid compound or composition of the present invention via inhalation is greater by about 1%, greater by about 2%, greater by about 3%, greater by about 4%, greater by about 5%, greater by about 6%, greater by about 7%, greater by about 8%, greater by about 9%, greater by about 10%, greater by about 11%, greater by about 12%, greater by about 13%, greater by about 14%, greater by about 15%, greater by about 16%, greater by about 17%, greater by about 18%, greater by about 19%, greater by about 20%, greater by about 25%, greater by about 30%, greater by about 35%, greater by about 40%, greater by about 45%, greater by about 50%, greater by about 55%, greater by about 60%, greater by about 65%, greater by about 70%, greater by about 75%, greater by about 80%, greater by about 85%, greater by about 90%, and all values in between compared to a FEV₁% of the patient prior to treatment.

In one embodiment, the stage of the chest radiograph of a patient administered a composition of the present invention via inhalation improves from stage 4 to stage 3, from stage 4 to stage 2, from stage 4 to stage 1, from stage 3 to stage 2, from stage 3 to stage 1, from stage 2 to stage 1, compared to the stage of a chest radiograph of the patient prior to treatment.

The skin is the second most affected organ in sarcoidosis, occurring in about 25% to 30% of cases. The most common lesions include erythema nodosum, plaques, maculopapular eruptions, subcutaneous nodules and lupus pernio. Some lesions spontaneously resolve within a few weeks. Skin lesions can be evaluated by a number of scoring systems for chronic facial lesions such as: the Sarcoidosis Activity and Severity Index (SASI) (Baughman et al. (2008) Am. J. of Clinical Dermatology 9, pp. 155-161, incorporated by reference herein in its entirety for all purposes), the Lupus Pernio Activity and Severity Index (LuPASI), a scoring system specific for this skin condition (Baughman et al. (2004) Chest Journal 126 (4_Meeting Abstracts): 891S, incorporated by reference herein in its entirety for all purposes) and the Cutaneous Sarcoidosis Activity and Morphology Instrument (CSAMI) (Rosenbach et al. 2013, JAMA Dermatology 149(5): 550-556, incorporated by reference herein in its entirety for all purposes).

SASI evaluates the following four features for each of the four facial quadrants and the nose: erythema, induration, and desquamation, each ranging from 0 (none) to 4 (very severe), and an area score ranging from 0 (0%) to 6 (90%400%). Thus, SASI produces 5 separate sets of scores per patient. The Facial SASI score weighs these SASI components to provide a composite index for the face. SASI can be modified and incorporated into clinical trials. For example, the sums of the erythema, induration, and desquamation scores for each quadrant of the face and the nose can be multiplied by their respective area scores and then averaged with equal weight on all 5 regions. The maximal range of the modified Facial SASI scores is 0 to 72.

The LuPASI is specific for scoring lupus pernio and is based on the psoriasis activity and severity index. The face is divided into specific areas and each area is separately scored on a five point scale for erythema (E), induration (I), and desquamation (D). The total amount of the area (A) involved is also assessed on a 7 point scale. The divisions are the four quadrants of the face, with the division of upper and lower being through the mid eye, and the nose is scored separately.

The CSAMI consists of 2 scores measuring disease activity and damage done by the disease. The Activity and Damage scales are considered separately to aid the instrument in detecting changes in disease activity, rather than remaining stable as a single conglomerate outcome as inflammatory activity subsides and chronic damage develops. Activity is scored based on inflammation, induration and/or depression, surface changes, such as scaling and ulceration, and area of involvement. Damage is scored based on dyspigmentation and scarring. Clinical signs are documented according to the worst affected lesion within each anatomical area and summed, with maximal score ranges of 0 to 165 for the Activity scale and 0 to 22 for the Damage scale. In addition, CSAMI assesses morphologic types of cutaneous sarcoidosis lesions, documenting a predominant type and all other types present. The instrument also examines the presence of lesion types that connote specific significance when present, including lupus pernio and erythema nodosum.

In one embodiment, the patient has cutaneous sarcoidosis in addition to pulmonary sarcoidosis. In another embodiment, administration of the effective amount of the disease-modifying antisarcoid compound, a derivative thereof, or pharmaceutically acceptable salt thereof, results in improved Sarcoidosis Activity and Severity Index (SASI), Lupus Pernio Activity and Severity Index (LuPASI) or Cutaneous Sarcoidosis Activity and Morphology Instrument (CSAMI) of the patient, as compared to the patient's SASI, LuPASI or CSAMI prior to treatment.

In one embodiment, the patient's SASI, LuPASI or CSAMI score improves by less than 1 point, by about 1 point, by about 2 points, by about 3 points, by about 4 points, by about 5 points, by about 6 points, by about 7 points, by about 8 points, by about 9 points, by about 10 points, or more, as compared to the patient's SASI, LuPASI or CSAMI score prior to treatment.

The compositions provided herein may also be used in combination with an enhancer agent and/or with a second active ingredient. In certain embodiments, the compounds are administered in combination in the same composition, or administered serially. Such other therapeutic agents include those known for treatment, prevention, or amelioration of one or more symptoms associated with sarcoidosis.

A compound of the present disclosure, or a pharmaceutically acceptable salt, prodrug, metabolite, analog or derivative thereof, may be administered in combination with a second disease-modifying antisarcoid compound. For example, as noted above, the compositions of the present disclosure may include the compounds as described above in combination with one or more (e.g., 1, 2, 3) additional active agents such as described in this section in analogous manner as known in the art.

In one embodiment, the additional disease-modifying antisarcoid compound includes, but is not limited to, a steroid compound. In one embodiment, the steroid compound is a corticosteroid compound. In a further embodiment, the corticosteroid compound is prednisone, prednisolone, flunisolide, fluticasone furoate, fluticasone propionate, triamcinolone acetonide, beclomethasone dipropionate, budesonide, dexamethasone, hydrocortisone, alcometasone, betamethasone, ciclesonide, clobetasol, deflazacort, diflucortolone, fludrocortisone, fluocinolone, fluometholone, fluticasone, mometasone, methylprednisolone, nandrolone decanoate, neomycin sulphate, rimexolone, triamcinolone, or a combination thereof.

In one embodiment, the additional disease-modifying antisarcoid active agent that may be used with the compounds of the present disclosure in carrying out the present invention includes, is not limited to, anti-interleukin neutralizing antbody and/or anti-interferon neutralizing antibody. An example of an anti-interleukin antibody includes ustekinumab.

The composition comprising a disease-modifying antisarcoid compound, derivative thereof, or pharmaceutically acceptable salt thereof, in one aspect of the invention, is packaged as a kit that further includes an inhalation device. The inhalation device may be disposable, single-use or a multiple-use device.

In another embodiment of the invention, the disease-modifying antisarcoid compound, derivative thereof, or pharmaceutically acceptable salt thereof used for treatment comprises an effective amount of methotrexate (MTX), azathioprine (AZA), leflunomide, mycophenolate mofetil, mycophenolic acid, chloroquine, hydroxychloroquine, cyclosporine, chlorambucil, thalidomide, cyclophosphamide and pentoxifylline, a derivative thereof (e.g., a prodrug thereof), or a pharmaceutically acceptable salt thereof.

In another embodiment, the inhalation device comprises a metered dose inhaler (MDI), a dry powder inhaler (DPI), soft mist inhaler or a nebulizer.

Any of the compounds discussed herein can be provided as a component of a composition. The composition, for example, can be administered to a patient in need of sarcoidosis treatment via inhalation.

The compositions described herein in one embodiment, are useful in methods for treating a patient for pulmonary sarcoidosis via inhalation delivery. As described throughout, the method entails administering to the lungs of a patient in need thereof a composition comprising an effective amount of a disease-modifying antisarcoid compound and a pharmaceutically acceptable inhalation excipient.

Compositions provided herein in one embodiment include one or more excipients, e.g., one or more pharmaceutically acceptable inhalation carriers or excipients together with a disease modifying antisarcoid compound (e.g., a compound of Formula I or II). The term “excipient” refers to a natural or synthetic substance formulated alongside the active ingredient of a medication, included for the purpose of bulking-up formulations that contain potent active ingredients (thus often referred to as “bulking agents,” “fillers,” or “diluents”), or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. The selection of appropriate excipients also depends upon the route of administration and the dosage form, as well as the active ingredient and other factors. Though excipients were at one time considered to be “inactive” ingredients, they are now understood to be integral to dosage form performance.

As used herein, “pharmaceutically acceptable inhalation carrier” may include any and all solvents, diluents, or other liquid vehicle, dispersion or suspension aids, surface active agents, isotonic agents, thickening or emulsifying agents, preservatives, solid binders, lubricants and the like, as suited to the inhalation dosage form provided herein. Remington's Pharmaceutical Sciences, Sixteenth Edition, E. W. Martin (Mack Publishing Co., Easton, Pa., 1980), incorporated by reference herein in its entirety for all purposes, discloses various carriers used in formulating pharmaceutical compositions and known techniques for the preparation thereof. Except insofar as any conventional carrier medium is incompatible with the compounds such as by producing any undesirable biological effect or otherwise interacting in a deleterious manner with any other component(s) of the pharmaceutical composition, its use is contemplated to be within the scope of this disclosure. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. “Pharmaceutically acceptable excipient or carrier” also relates to an excipient or carrier that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

Such formulations may include an antioxidant, such as acetone sodium bisulfate, ascorbic acid; preservatives, such as ammonia, benzalkonium chloride, cetylpyridinium chloride, chlorobutanol, glycerin, methylparaben, propylparaben, propylene glycol, sodium metabisulfite, sodium sulfite; wetting, emulsification, dispersion, solubilization agents, suspension aids and valve lubricants such as benzalkonium chloride, lecithin (soya), magnesium stearate, oleic acid, polysorbate 80, polyvinylpyrrolidone K25, sorbitan trioleate (Span 85), Thymol, Pluronic® F-77, Pluronic® F-68, Pluronic® L-92, Pluronic® L-121, polyethylene glycol, diethylene glycol monoethyl ether, polyoxyethylene sorbitan monolaurate, polyoxyethylene sorbitan monooleate, propoxylated polyethylene glycol, and polyoxyethylene lauryl ether, methyl polyethylene glycol (f-mPEG), oligolactic acid (OLA), hydrophobic counterions (e.g., lauric acid, lauroyl sarcosine and lauroyl lactylate) and hydrophilic counterions (e.g., functionalized polyethers), acetylated cyclodextrins; flavorings, such as citric acid (anhydrous), menthol, saccharin, saccharin sodium dehydrate, sodium citrate; chelating agents, such as edetate sodium/edetate disodium, sodium citrate; cosolvents, such as ethanol, dehydrated alcohol, alcohol, glycerin, propylene glycol, water; humectants, such as glycerin; tonicity agents, such as glycerin, sodium chloride, sodium sulfate (anhydrous); buffering agents, such as glycine, lysine monohydrate, sodium citrate, tromethamine; drug stabilizers, such as glycine, lysine monohydrate; pH adjustors, such as hydrochloric acid, nitric acid, sodium bisulfate, sodium hydroxide, sulfuric acid.

In one embodiment, the pharmaceutically acceptable inhalation carrier or excipient is used to facilitate delivery by a particular inhalation delivery device. For example, in the case of a composition delivered via a metered dose inhaler, a propellant is provided in the composition. In a further embodiment, the composition comprises an effective amount of a disease-modifying antisarcoid compound, a derivative thereof, or a pharmaceutically acceptable salt thereof, and a chlorofluorocarbon free propellant. In one embodiment, the chlorofluorocarbon free propellant is hydrofluoroalkane (HFA)-134a, HFA-227, HFA-152 (difluoroethane and isobutene), trans-1,3,3,3,-tetrafluoropro-1-ene (HFO 1234ze), 2,3,3,3,-tetrafluoroprop-1-ene (HFO 1234yf), or combinations thereof.

In another embodiment, the composition comprises an effective amount of a disease-modifying antisarcoid compound, a derivative thereof (e.g., a prodrug thereof), or a pharmaceutically acceptable salt thereof and a solubilization agent. In a further embodiment, the solubilization agent is Pluronic® F-77, Pluronic® F-68, Pluronic® L-92, Pluronic® L-121, polyethylene glycol, diethylene glycol monoethyl ether, polyoxyethylene sorbitan monolaurate, polyoxyethylene sorbitan monooleate, propoxylated polyethylene glycol, polyoxyethylene lauryl ether, methyl polyethylene glycol (f-mPEG), oligolactic acid (OLA), hydrophobic counterions, hydrophilic counterions, acetylated cyclodextrins, or combinations thereof.

In yet another embodiment, the composition comprises an effective amount of a disease-modifying antisarcoid compound, a derivative thereof, or a pharmaceutically acceptable salt thereof and a suspension aid. In another embodiment, the suspension aid is oleic acid, polysorbate 80, polyvinylpyrrolidone K25, or combinations thereof.

Compositions provided herein can be formulated as dry powders, solutions and suspensions.

In one aspect of the invention, the disease-modifying antisarcoid compound described herein, e.g., a compound of Formula I or II, is provided in a composition comprises a disease-modifying antisarcoid compound, derivative thereof, or pharmaceutically acceptable salt thereof complexed to or encapsulated by a lipid component. The composition comprising the lipid component and the disease-modifying antisarcoid compound in one embodiment is administered to a subject in need of sarcoidosis treatment via one of the inhalation delivery methods described herein. In one embodiment, the lipid component is present in solid lipid nanoparticles. The complex, in one embodiment, is formed by one or more electrostatic interactions, hydrophobic interactions, hydrogen bonds or by the encapsulation of the disease-modifying antisarcoid compound by the lipid, e.g., in a micelle or liposome. In another embodiment, the lipid component comprises liposomes. For example, the lipid-complexed composition, in one embodiment, comprises liposomes, and the disease-modifying antisarcoid compound may be in the aqueous phase (encapsulated by the liposome), the hydrophobic bilayer phase, at the interfacial headgroup region of the liposomal bilayer or a combination thereof.

The lipid component can comprise a homogeneous population of lipid or a heterogeneous population of lipid. That is, different lipids can be employed in the same composition, if desired. The lipid component is complexed to a disease-modifying antisarcoid compound. The complex, in one embodiment, is a microparticle, nanoparticle, micelle or liposome, or a combination thereof. In a further embodiment, the composition comprises a cationic lipid, or a mixture of different cationic lipids complexed to a disease-modifying antisarcoid compound.

In one embodiment, the lipid complex is a liposome or liposomes, and the disease-modifying antisarcoid compound is associated within the liposome surface or present in the aqueous interior of the liposome (or liposomes). Liposomes are completely closed lipid bilayer membranes containing an entrapped aqueous volume. Liposomes may be unilamellar vesicles (possessing a single membrane bilayer) or multilamellar vesicles (onion-like structures characterized by multiple membrane bilayers, each separated from the next by an aqueous layer) or a combination thereof. The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “heads” orient towards the aqueous phase.

In one embodiment, when formulated together, the disease-modifying antisarcoid compound and lipid component is present in lipid particles (e.g., microparticles or nanoparticles). In one embodiment, the lipid component is a cationic lipid, a PEGylated lipid, a surfactant or a block copolymer.

In some embodiments, the lipid component of the composition comprises a lipid selected from the group consisting of: a cationic lipid, an anionic lipid, a neutral lipid, a conjugated lipid, and mixtures thereof. For example, in one embodiment, the lipid component comprises a mixture of one or more cationic lipids and one or more neutral lipids. In another embodiment, the lipid component comprises a mixture of one or more cationic lipids, one or more neutral lipids, and one or more conjugated lipids. In yet another embodiment, the lipid component comprises a mixture of one or more cationic lipids, one or more anionic lipids, one or more neutral lipids, and one or more conjugated lipids.

In one embodiment, the neutral lipid present in the compositions of the invention comprises a mixture of two or more neutral lipids. Neutral lipids include, but are not limited to, phospholipids such as phosphatidylcholines and phosphatidylethanolamines, ceramide, sphingomyelin, cephalin, sterols such as cholesterol or derivatives thereof, tocopherols (e.g. methylated phenols many of which have vitamin E activity) or derivatives thereof, cerebrosides, and diacylglycerols.

In a particular embodiment, the lipid component of the composition comprises a conjugated lipid. In another particular embodiment, the lipid component of the composition consists of a conjugated lipid.

In one embodiment, the lipid component of the compositions of the invention comprises or consists of a conjugated lipid. The term “conjugated lipid” refers to a lipid that is coupled to a non-lipid moiety. Such conjugated lipids include, but are not limited to, polyethylene glycol (PEG)-lipid conjugates and methoxypolyethylene glycol (MPEG)-lipid conjugates, i.e., conjugated lipid is a PEGylated lipid or MPEGylated lipid. PEG or MPEG can be conjugated directly to the lipid or may be linked to the lipid via a linker moiety. Any linker moiety suitable for coupling the PEG or MPEG to a lipid can be used including, e.g., non-ester containing linker moieties and ester-containing linker moieties. The general formula for PEG is: H—(OCH₂CH₂)ₙ—OH and the general formula for MPEG is: CH₃—(OCH₂CH₂)ₙ—OH where “n” is the average number of repeating oxyethylene groups.

In one embodiment, the conjugated lipid is a PEGylated lipid. The PEGylated lipid, in one embodiment, comprises PEG400-PEG5000. For example, the PEGylated lipid can comprise PEG400, PEG500, PEG1000, PEG2000, PEG3000, PEG4000, or PEG5000. In a further embodiment the lipid component of the PEGylated lipid comprises cholesterol, dimyristoyl phosphatidylethanolamine (DMPE), dipalmitoyl phosphoethanolamine (DPPE), distearoylphosphatidylethanolamine (DSPE), dimyristoylglycerol glycerol (DMG), diphosphatidylglycerol (DPG) or disteraroylglycerol (DSG). In some embodiments, the PEGylated lipid is DMG-PEG2000, cholesterol-PEG2000 or DSPE-PEG2000.

Exemplary PEG-lipid conjugates include PEG coupled to dialkyloxypropyls, PEG coupled to diacylglycerols, PEG coupled to cholesterol, PEG coupled to phosphatidylethanolamines, PEG conjugated to ceramides (see, e.g., U.S. Pat. No. 5,885,613, the disclosure of which is herein incorporated by reference in its entirety for all purposes), cationic PEG lipids, and mixtures thereof.

In another embodiment, the conjugated lipid is a MPEGylated lipid. The MPEGylated lipid, in one embodiment, comprises MPEG400-MPEG5000. For example, the MPEGylated lipid can comprise MPEG400, MPEG500, MPEG1000, MPEG2000, MPEG3000, MPEG4000, or MPEG5000. In a further embodiment the lipid component of the MPEGylated lipid comprises cholesterol, dimyristoyl phosphatidylethanolamine (DMPE), dipalmitoyl phosphoethanolamine (DPPE), distearoylphosphatidylethanolamine (DSPE), dimyristoylglycerol glycerol (DMG), diphosphatidylglycerol (DPG) or disteraroylglycerol (DSG). In some embodiments, the MPEGylated lipid is DMG-MPEG2000, cholesterol-MPEG2000 or DSPE-MPEG2000.

Exemplary MPEG-lipid conjugates include MPEG coupled to dialkyloxypropyls, MPEG coupled to diacylglycerols, MPEG coupled to cholesterol, MPEG coupled to phosphatidylethanolamines, MPEG conjugated to ceramides, cationic MPEG lipids, and mixtures thereof. In an exemplary embodiment, the conjugated lipid is DMG-MPEG2000.

The conjugated lipid, for example the PEGylated lipid or MEPGylated lipid, can have a net-charge (e.g., cationic or anionic), or can be net-neutral. The lipids used in the PEGylated/MPEGylated lipid component of the present invention can be synthetic, semi-synthetic or naturally-occurring lipid, including a phospholipid, a sphingolipid, a glycolipid, a ceramide, a tocopherol, a sterol, a fatty acid, or a glycoprotein such as albumin. In one embodiment, the lipid is a sterol. In a further embodiment, the sterol is cholesterol. In another embodiment, the lipid is a phospholipid described herein. In various embodiments, the PEGylated/MEPGylated lipid of the composition provided herein comprises distearoylphosphoethanolamine (DSPE), dipalmitoylphosphatidylcholine (DPPC), dioleoylphosphatidylcholine (DOPC) dimyristoyl phosphatidylethanolamine (DMPE), dipalmitoylphosphoethanolamine (DPPE), distearoylphosphatidylethanolamine (DSPE), dimyristoylglycerol (DMG), diphosphatidylglycerol (DPG) or disteraroylglycerol (DSG).

In various embodiments, the conjugated lipid comprises about 95, 90, 85, 80, 75, 70, 65, 60, 55, 50, 45, 40, 35, 30, 25, 20, 15, 10, or 5 mol % or w/w %, including values and subranges therebetween, of the total lipid present in the composition. In some embodiments, the conjugated lipid comprises about 50-90, 60-90, 70-90, 80-90 mol % or w/w %, including values and subranges therebetween, of the total lipid present in the composition. In an exemplary embodiment, the conjugated lipid is DMG-MPEG2000 and comprises about 80-90 mol % of the total lipid of the composition.

The lipid component can comprise a negatively charged lipid, a positively charged lipid, a net neutral lipid, or a combination thereof. For example, in one embodiment of a composition described herein, the lipid component is an electrically neutral lipid selected from the group consisting of egg phosphatidylcholine (EPC), phosphatidylethanolamine (EPE), phosphatidic acid (EPA), soy phosphatidylcholine (SPC), soy phosphatidylethanolamine (SPE), hydrogenated egg phosphatidylcholine (HEPC), hydrogenated phosphatidylethanolamine (HEPE), hydrogenated soy phosphatidylcholine (HSPC), hydrogenated soy phosphatidylethanolamine (HSPE), dipalmitoylphosphatidylcholine (DPPC), dimyristoylphosphatidylcholine (DMPC), distearoylphosphatidylcholine (DSPC), 1,2-Oleoyl-sn-glycero-3-phosphocholine (DOPC), dioleylphosphatidyl-ethanolamine (DOPE), palmitoylstearoylphosphatidyl-choline (P SPC), mono-oleoyl-phosphatidylethanolamine (MOPE) and tocopherol. In another embodiment, the lipid component comprises a phosphatidylcholine, a sterol, a phospholipid, a tocopherol, a fatty acid, a synthetic lipid, a semi-synthetic lipid, or a mixture thereof.

In some embodiments, the lipid component of the composition comprises a lipid selected from the group consisting of: a cationic lipid, an anionic lipid, a phospholipid, a sterol, a tocopherol, a conjugated lipid, and mixtures thereof. For example, in one embodiment, the lipid component comprises a mixture of one or more cationic lipids and one or more phospholipids. In another embodiment, the lipid component comprises a mixture of one or more cationic lipids, one or more phospholipids, and a sterol or a derivative thereof and optionally comprises a conjugated lipid. In yet another embodiment, the lipid component comprises a mixture of one or more cationic lipids, one or more phospholipids, and a tocopherol or a derivative thereof and optionally comprises a conjugated lipid.

In yet another embodiment, the lipid component of the composition comprises a mixture of one or more cationic lipids, one or more anionic lipids, and one or more phospholipids and optionally comprises a conjugated lipid. In yet another embodiment, the lipid component comprises a mixture of one or more cationic lipids, one or more anionic lipids, one or more phospholipids, and a sterol or a derivative thereof and optionally comprises a conjugated lipid. In yet another embodiment, the lipid component comprises a mixture of one or more cationic lipids, one or more anionic lipids, one or more phospholipids, and a tocopherol or a derivative thereof and optionally comprises a conjugated lipid.

In some embodiments, the lipid component of the composition comprises a mixture of one or more phospholipids and a sterol or a derivative thereof and optionally comprises a conjugated lipid. For example, in one embodiment, the lipid component of the composition comprises a phophatidylcholine (e.g. DPPC, DMPC, DOPC, DSPC, and PSPC) and cholesterol or a derivative thereof and optionally comprises a conjugated lipid. In another embodiment, the lipid component of the composition consists of a phophatidylcholine (e.g. DPPC, DMPC, DOPC, DSPC, and PSPC) and cholesterol or a derivative thereof.

In some embodiments, the lipid component of the composition comprises a mixture of one or more phospholipids and a tocopherol or a derivative thereof and optionally comprises a conjugated lipid. For example, in one embodiment, the lipid component of the composition comprises a phophatidylcholine (e.g. DPPC, DMPC, DOPC, DSPC, and PSPC) and tocopherol or a derivative thereof and optionally comprises a conjugated lipid. In another embodiment, the lipid component of the composition consists of a phophatidylcholine (e.g. DPPC, DMPC, DOPC, DSPC, and PSPC) and tocopherol or a derivative thereof.

In some embodiments, the compositions and/or lipid particles of the invention are free of anionic lipids (negatively charged lipid). However, if an anionic lipid is present, such lipids include phosphatidyl-glycerols (PGs), phosphatidic acids (PAs), phosphatidylinositols (Pis) and the phosphatidyl serines (PSs). Examples include DMPG, DPPG, DSPG, DMPA, DPPA, DSPA, DMPI, DPPI, DSPI, DMPS, DPPS and DSPS.

As provided above, in one embodiment, a cationic lipid is provided in the composition described herein together with a disease-modifying antisarcoid compound. In various embodiments, the cationic lipid comprises about 5, 10, 15, 20, 25, 30, 35, 40, 45, 50, 55, 60, 65, 70, 75, 80, 85, 90, or 95 mol % or w/w %, including values and subranges therebetween, of the total lipid present in the composition. For example, in certain embodiments, the cationic lipid comprises about 5-95, about 10-90, about 15-85, about 20-80, about 25-75, about 30-70, about 35-65, about 40-60, about 5-80, about 5-70, about 5-60, about 5-50, about 5-40, about 5-30, about 5-20, about 10-80, about 10-70, about 10-60, about 10-50, about 10-40, about 10-30, about 20-80, about 20-70, about 20-60, about 20-50, about 20-40, about 30-80, about 30-70, about 30-60, about 30-50, about 40-80, about 40-70, about 40-60, about 50-80, or about 50-70 mol % or w/w %, including values and subranges therebetween, of the total lipid present in the composition.

As provided above, in one embodiment, a cationic lipid is provided in the composition described herein together with a disease-modifying antisarcoid compound. The cationic lipid, in one embodiment, includes ammonium salts of fatty acids, phospholipids and glycerides. The fatty acids include fatty acids of carbon chain lengths of 12 to 26 carbon atoms that are either saturated or unsaturated. Some specific examples include: myristylamine, palmitylamine, laurylamine and stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadec enyl oxy)-prop-1-yl-N,N,N-trimethylammonium chloride (DOTMA), dioleylphosphatidylethanolamine (DOPE) and 1,2-bis(oleoyloxy)-3-(trimethylammonio) propane (DOTAP).

In one embodiment, the lipid component is designed to target a mononuclear phagocyte, for example a monocyte or macrophage. In a further embodiment, the mononuclear phagocyte is a macrophage. The lipid component, for example, comprises a negatively charged lipid, for example, a negatively charged phospholipid. In one embodiment, the negatively charged phospholipid is a phosphatidylserine (PS) and/or phosphatidylglycerol (PG). The phosatidylserine and/or phosphatidylglycerol can be any phosphatidylserine known to those of ordinary skill in the art. For example, the PS in one embodiment is egg phosphatidylserine (EPS), dilauroyl-phosphoserine (DLPS), dimyristoylphosphoserine (DMPS), dioleoyl-phosphoserine (DOPS), dipalmitoyl-phosphoserine (DPPS), distearoyl-phosphoserine (DSPS) or a combination thereof. The PG, in one embodiment, is egg phosphatidylglycerol (EPG), dipalmitoylphosphatidylglycerol (DPPG), dioleoyl-glycero-phosphatidylglycerol (DOPG), dimyristoylphosphatidylglycerol (DMPG), distearoylphosphatidylglycerol (DSPG), palmitoyl-oleoyl-phosphatidylglycoerol (POPG), or a combination thereof.

As provided above, combinations of negatively charged lipids can also be employed. Without wishing to be bound by theory, it is thought that the negatively charged lipid (or combination thereof) of the lipid component targets a mononuclear phagocyte (e.g., monocyte or macrophage) by interaction with scavenger receptors on the mononuclear phagocyte's cell surface.

In one embodiment, the lipid component comprises one or more negatively charged lipids and one or more net neutral lipids, for example, a net neutral phospholipid, cholesterol or a combination thereof. The net neutral phospholipid in one embodiment is a phosphatidylcholine. In a further embodiment, the phosphatidylcholine is egg phosphatidylcholine, dipalmitoylphosphatidylcholine (DPPC), distearoylphosphatidylcholine (DSPC), 1,2-Oleoyl-sn-glycero-3-phosphocholine (DOPC), dimyristoylphosphatidylcholine (DMPC), lysolecithin or a combination thereof.

In one embodiment, the lipid component comprises a negatively charged lipid and a glycerol based phospholipid and/or a glycosphingolipid. In a further embodiment, the glycerol based phospholipid is a phosphatidate (or the acid form as a phosphatidic acid). In one embodiment the glycosphingolipid is a ganglioside.

Liposomes targeting mononuclear phagocytes have been investigated previously and are amenable for use with the invention described herein. For example, the lipids and liposomes described by the following references, each of which is incorporated by reference herein in their entireties, can be employed as the lipid component of the present invention: Fidler et al. (1980). Cancer Res. 40, pp. 4460-4466; Schroit and Fidler (1982). Cancer Res. 42, pp. 161-167; Bakker-Woudenberg et al. (1988). Antimicrobial Agents and Chemotherapy 32, pp. 1560-1564; Fidler (1988). Advanced Drug Delivery Reviews 2, pp. 69-106; Oussoren et al. (1997). Biochimica et Biophysica Acta 1328, pp. 261-272; Kelly et al. (2011). Journal of Drug Delivery 2011, Article 727241, doi: 10.1155/2011/727241. Combinations of lipids for use in certain embodiments of the invention are provided in Table 1 below.

TABLE 1

| Lipid component | | Lipid component | |
|---|---|---|---|
| 1. | Negatively charged phospholipid | 2. | Negatively charged phospholipid/net neutral |
| | | | lipid (e.g., cholesterol or net neutral |
| | | | phosphatidylcholine such as DPPC, DSPC, |
| | | | DOPC and/or DMPC) |
| 3. | Phosphatidylgyclerol (PG) | 4. | Phosphatidylgyclerol (PG)/net neutral lipid (e.g., |
| | | | cholesterol or net neutral phosphatidylcholine |
| | | | such as DPPC, DSPC, DOPC and/or DMPC) |
| 5. | egg phosphatidylglycerol (EPG) | 6. | egg phosphatidylglycerol (EPG)/net neutral lipid |
| | | | (e.g., cholesterol or net neutral |
| | | | phosphatidylcholine such as DPPC, DSPC, |
| | | | DOPC and/or DMPC) |
| 7. | dipalmitoylphosphatidylglycerol | 8. | dipalmitoylphosphatidylglycerol (DPPG)/net |
| | (DPPG) | | neutral lipid (e.g., cholesterol or net neutral |
| | | | phosphatidylcholine such as DPPC, DSPC, |
| | | | DOPC and/or DMPC) |
| 9. | dioleoyl-glycero- | 10. | dioleoyl-glycero-phosphatidylglycerol (DOPG)/ |
| | phosphatidylglycerol (DOPG) | | net neutral lipid (e.g., cholesterol or net neutral |
| | | | phosphatidylcholine such as DPPC, DSPC, |
| | | | DOPC and/or DMPC) |
| 11. | dimyristoylphosphatidylglycerol | 12. | dimyristoylphosphatidylglycerol (DMPG)/net |
| | (DMPG) | | neutral lipid (e.g., cholesterol or net neutral |
| | | | phosphatidylcholine such as DPPC, DSPC, |
| | | | DOPC and/or DMPC) |
| 13. | distearoylphosphatidylglycerol | 14. | distearoylphosphatidylglycerol (DSPG)/net |
| | (DSPG), | | neutral lipid (e.g., cholesterol or net neutral |
| | | | phosphatidylcholine such as DPPC, DSPC), |
| | | | DOPC and/or DMPC) |
| 15. | palmitoyl-oleoyl- | 16. | palmitoyl-oleoyl-phosphatidylglycoerol (POPG)/ |
| | phosphatidylglycoerol (POPG) | | net neutral lipid (e.g., cholesterol or net neutral |
| | | | phosphatidylcholine such as DPPC, DSPC, |
| | | | DOPC and/or DMPC) |
| 17. | phosphatidylserine (PS) | 18. | phosphatidylserine (PS)/net neutral lipid (e.g., |
| | | | cholesterol or net neutral phosphatidylcholine |
| | | | such as DPPC, DSPC, DOPC and/or DMPC) |
| 19. | egg phosphatidylserine (EPS) | 20. | egg phosphatidylserine (EPS)/net neutral lipid |
| | | | (e.g., cholesterol or net neutral |
| | | | phosphatidylcholine such as DPPC, DSPC, |
| | | | DOPC and/or DMPC) |
| 21. | dilauroyl-phosphoserine (DLPS) | 22. | dilauroyl-phosphoserine (DLPS)/net neutral |
| | | | lipid (e.g., cholesterol or net neutral |
| | | | phosphatidylcholine such as DPPC, DSPC, |
| | | | DOPC and/or DMPC) |
| 23. | dimyristoylphosphoserine (DMPS) | 24. | dimyristoylphosphoserine (DMPS)/net neutral |
| | | | lipid (e.g., cholesterol or net neutral |
| | | | phosphatidylcholine such as DPPC, DSPC, |
| | | | DOPC and/or DMPC) |
| 25. | dioleoyl-phosphoserine (DOPS) | 26. | dioleoyl-phosphoserine (DOPS)/net neutral lipid |
| | | | (e.g., cholesterol or net neutral |
| | | | phosphatidylcholine such as DPPC, DSPC, |
| | | | DOPC and/or DMPC) |
| 27. | dipalmitoyl-phosphoserine (DPPS) | 28. | dipalmitoyl-phosphoserine (DPPS)/net neutral |
| | | | lipid (e.g., cholesterol or net neutral |
| | | | phosphatidylcholine such as DPPC, DSPC, |
| | | | DOPC and/or DMPC) |
| 29. | PS/cholesterol/phosphatidylcholine | 30. | PS/chol/DPPC, DSPC, DOPC and/or DMPC |
| 31. | PG/cholesterol/phosphatidylcholine | 32. | PG/chol/DPPC, DSPC, DOPC and/or DMPC |
| 33. | PS/PG/phosphatidylcholine | 34. | PS/PG/chol/DPPC, DSPC, DOPC and/or DMPC |

Other examples of lipids for use in lipid components provided herein (PEGylated or non-PEGylated) include dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG) dioleylphosphatidylethanolamine (DOPE), and mixed phospholipids such as palmitoylstearoylphosphatidylcholine (PSPC) and palmitoylstearoylphosphatidylglycerol (PSPG), triacylglycerol, diacylglycerol, ceramide, sphingosine, sphingomyelin and single acylated phospholipids such as mono-oleoyl-phosphatidylethanolamine (MOPE). In another embodiment lipid component of the composition comprises an ammonium salt of a fatty acid, a phospholipid, a glyceride, a phospholipid and glyceride, a sterol (e.g., cholesterol), phosphatidylglycerol (PG), phosphatidic acid (PA), a phosphatidylcholine (PC), a phosphatidylinositol (PI), a phosphatidylserine (PS), or a combination thereof. The fatty acid, in one embodiment, comprises fatty acids of carbon chain lengths of 12 to 26 carbon atoms that are either saturated or unsaturated. Some specific examples include: myristylamine, palmitylamine, laurylamine and stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9(Z)-octadecenyl oxy)-prop-1-yl-N,N,N-trimethylammonium chloride (DOTMA) and 1,2-bis(oleoyloxy)-3-(trimethylammonio)propane (DOTAP). Examples of sterols for use in the lipid particle compositions provided herein include cholesterol and ergosterol. Examples of PGs, PAs, PIs, PCs and PSs for use in the compositions provided herein include DMPG, DPPG, DSPG, DMPA, DPPA, DSPA, DMPI, DPPI, DSPI, DMPS, DPPS and DSPS, DSPC, DPPG, DMPC, DOPC, egg PC and soya PC.

In yet another embodiment, two or more of the disease-modifying antisarcoid compounds, a lipid component (e.g., a cationic lipid, PEGylated lipid, MEGylated lipid, a phospholipid, a sterol, or combination thereof) and a hydrophobic additive are provided in a composition, for example, a composition comprising microparticles or nanoparticles of disease-modifying antisarcoid compound complexed to the lipid component.

In one lipid particle embodiment, the disease-modifying antisarcoid compound is present in the composition at 5 mol %-99 mol %. In a further embodiment, the compound is present in the composition at 40 mol %-95 mol %. In a further embodiment, the disease-modifying antisarcoid compound is present in the composition at 40 mol %-60 mol %. In one embodiment, the disease-modifying antisarcoid compound present in the composition at about 40 mol % or about 45 mol %.

In some embodiments, the compositions, systems and methods provided herein comprise a lipid complexed (e.g., liposomal encapsulated) disease-modifying antisarcoid compound. The lipids used in the pharmaceutical compositions of the present invention as provided throughout can be synthetic, semi-synthetic or naturally-occurring lipids, including phospholipids, tocopherols, sterols, fatty acids, negatively-charged lipids and cationic lipids. As provided above, where disease-modifying antisarcoid compounds are employed, cationic lipids or anionic lipids can be complexed thereto via electrostatic interactions.

In one embodiment, at least one phospholipid is present in the composition. In a further embodiment, the composition comprises liposomes or lipid particles comprising a lipid complexed disease-modifying antisarcoid compound. In one embodiment, the phospholipid is selected from: phosphatidylcholine (EPC), phosphatidylglycerol (PG), phosphatidylinositol (PI), phosphatidylserine (PS), phosphatidylethanolamine (PE), and phosphatidic acid (PA); the soya counterparts, soy phosphatidylcholine (SPC); SPG, SPS, SPI, SPE, and SPA; the hydrogenated egg and soya counterparts (e.g., HEPC, HSPC), phospholipids made up of ester linkages of fatty acids in the 2 and 3 of glycerol positions containing chains of 12 to 26 carbon atoms and different head groups in the 1 position of glycerol that include choline, glycerol, inositol, serine, ethanolamine, as well as the corresponding phosphatidic acids. The carbon chains on these fatty acids can be saturated or unsaturated, and the phospholipid may be made up of fatty acids of different chain lengths and different degrees of unsaturation.

In one embodiment, the composition includes dipalmitoylphosphatidylcholine (DPPC), a major constituent of naturally-occurring lung surfactant. In one embodiment, the lipid component of the composition comprises DPPC and cholesterol, or consists essentially of DPPC and cholesterol, or consists of DPPC and cholesterol. In a further embodiment, the DPPC and cholesterol have a mole ratio in the range of from about 19:1 to about 1:1, or about 9:1 to about 1:1, or about 4:1 to about 1:1, or about 2:1 to about 1:1, or about 1.86:1 to about 1:1. In even a further embodiment, the DPPC and cholesterol have a mole ratio of about 2:1 or about 1:1.

Without wishing to be bound by theory, phosphatidylcholines, such as DPPC, aid in the uptake of the antisarcoid compound by the cells in the lung (e.g., the alveolar macrophages) and helps to maintain the antisarcoid compound in the lung. The negatively charged lipids such as the PGs, PAs, PSs and PIs, in addition to reducing particle aggregation, are thought to play a role in the sustained activity characteristics of the inhalation formulation as well as in the transport of the formulation across the lung (transcytosis) for systemic uptake. The sterol compounds, without wishing to be bound by theory, are thought to affect the release characteristics of the formulation.

Other examples of lipids for use with the lipid complexed (e.g., liposomal, micelle, lipid particle) compositions described herein include but are not limited to, dimyristoylphosphatidycholine (DMPC), dimyristoylphosphatidylglycerol (DMPG), dipalmitoylphosphatidcholine (DPPC), dipalmitoylphosphatidylglycerol (DPPG), distearoylphosphatidylcholine (DSPC), distearoylphosphatidylglycerol (DSPG), dioleylphosphatidyl-ethanolamine (DOPE), mixed phospholipids such as palmitoylstearoylphosphatidyl-choline (PSPC), and single acylated phospholipids, for example, mono-oleoyl-phosphatidylethanolamine (MOPE). The one or more lipids, as described above, can be PEGylated.

In various embodiments, the phospholipid comprises about 5, 10, 15, 20, 25, 30, 35, 40, 45, 50, 55, 60, 65, 70, 75, 80, 85, 90, or 95 mol % or w/w %, including values and subranges therebetween, of the total lipid present in the composition. For example, in certain embodiments, the phospholipid comprises about 5-95, about 10-90, about 15-85, about 20-80, about 25-75, about 30-70, about 35-65, about 40-60, about 5-80, about 5-70, about 5-60, about 5-50, about 5-40, about 5-30, about 5-20, about 10-80, about 10-70, about 10-60, about 10-50, about 10-40, about 10-30, about 20-80, about 20-70, about 20-60, about 20-50, about 20-40, about 30-80, about 30-70, about 30-60, about 30-50, about 40-80, about 40-70, about 40-60, about 50-80, or about 50-70 mol % or w/w %, including values and subranges therebetween, of the total lipid present in the composition.

In one embodiment, the lipid component of the liposomal or lipid particle composition comprises a sterol. In a further embodiment, the lipid component of the liposomal composition comprises a sterol and a phospholipid, or consists essentially of a sterol and a phospholipid, or consists of a sterol and a phospholipid. Sterols for use with the invention include, but are not limited to, cholesterol, esters of cholesterol including cholesterol hemi-succinate, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate and lanosterol sulfate.

In some embodiments, the lipid component of the invention includes methylated phenols, such as tocopherols. In one embodiment, the lipid component includes methylated phenols with vitamin E activity, e.g. α-tocopherol. The tocopherols for use with the invention include tocopherols, esters of tocopherols including tocopherol hemi-succinates (e.g. α-tocopherol hemi-succinate), salts of tocopherols including tocopherol hydrogen sulfates and tocopherol sulfates. PCT Publication No. WO 85/00968, incorporated by reference in its entirety, describes a method for reducing the toxicity of drugs by encapsulating them in liposomes comprising α-tocopherol and certain derivatives thereof. Also, a variety of tocopherols and their water soluble derivatives have been used to form liposomes, see PCT Publication No. 87/02219, incorporated by reference in its entirety. The methods described in these publications are amenable for use herein.

Liposomes can be produced by a variety of methods and the present invention is not limited to a particular type of liposomal manufacturing method. In one embodiment, one or more of the methods described in U.S. Patent Application Publication No. 2008/0089927 or WO 2013/177226 are used herein to produce the disease-modifying antisarcoid compound encapsulated lipid compositions (liposomal dispersion). The disclosures of U.S. Patent Application Publication No. 2008/0089927 and PCT publication no. 2013/177226 are incorporated by reference in their entireties for all purposes.

In one embodiment, the liposomal composition is formed by dissolving one or more lipids in an organic solvent forming a lipid solution, and the disease-modifying antisarcoid compound coacervate forms from mixing an aqueous solution of the disease-modifying antisarcoid compound with the lipid solution. In a further embodiment, the organic solvent is ethanol. In even a further embodiment, the one or more lipids comprise a phospholipid and a sterol. The phospholipid, in one embodiment is net neutral or net cationic.

In one embodiment, liposomes are produces by sonication, extrusion, homogenization, swelling, electroformation, inverted emulsion or a reverse evaporation method. Bangham's procedure (J. Mol. Biol. (1965)) produces ordinary multilamellar vesicles (MLVs). Lenk et al. (U.S. Pat. Nos. 4,522,803, 5,030,453 and 5,169,637, each incorporated by reference in their entireties for all purposes), Fountain et al. (U.S. Pat. No. 4,588,578, incorporated by reference in its entirety) and Cullis et al. (U.S. Pat. No. 4,975,282, incorporated by reference in its entirety) disclose methods for producing multilamellar liposomes having substantially equal interlamellar solute distribution in each of their aqueous compartments. U.S. Pat. No. 4,235,871, incorporated by reference in its entirety, discloses preparation of oligolamellar liposomes by reverse phase evaporation. Each of the methods is amenable for use with the present invention.

Unilamellar vesicles can be produced from MLVs by a number of techniques, for example, the extrusion techniques of U.S. Pat. No. 5,008,050 and U.S. Pat. No. 5,059,421, the disclosure of each of which is incorporated by reference herein for all purposes. Sonication and homogenization cab be so used to produce smaller unilamellar liposomes from larger liposomes (see, for example, Paphadjopoulos et al. (1968); Deamer and Uster (1983); and Chapman et al. (1968), each of which is incorporated by reference in its entirety for all purposes).

The liposome preparation of Bangham et al. (J. Mol. Biol. 13, 1965, pp. 238-252, incorporated by reference herein in its entirety) involves suspending phospholipids in an organic solvent which is then evaporated to dryness leaving a phospholipid film on the reaction vessel. Next, an appropriate amount of aqueous phase is added, the 60 mixture is allowed to “swell,” and the resulting liposomes which consist of multilamellar vesicles (MLVs) are dispersed by mechanical means. This preparation provides the basis for the development of the small sonicated unilamellar vesicles described by Papahadjopoulos et al. (Biochim. Biophys. Acta. 135, 1967, pp. 624-638, incorporated by reference herein in its entirety), and large unilamellar vesicles.

Techniques for producing large unilamellar vesicles (LUVs), such as, reverse phase evaporation, infusion procedures, and detergent dilution, can be used to produce liposomes for use in the pharmaceutical compositions provided herein. A review of these and other methods for producing liposomes may be found in the text Liposomes, Marc Ostro, ed., Marcel Dekker, Inc., New York, 1983, Chapter 1, which is incorporated herein by reference. See also, Szoka, Jr. et al., (Ann. Rev. Biophys. Bioeng. 9, 1980, p. 467), which is also incorporated herein by reference in its entirety for all purposes.

Other techniques for making liposomes amenable for making the compositions described herein include those that form reverse-phase evaporation vesicles (REV), see, e.g., U.S. Pat. No. 4,235,871, incorporated by reference in its entirety. Another class of liposomes that may be used is characterized as having substantially equal lamellar solute distribution. This class of liposomes is denominated as stable plurilamellar vesicles (SPLV) as defined in U.S. Pat. No. 4,522,803, incorporated by reference in its entirety, and includes monophasic vesicles as described in U.S. Pat. No. 4,588,578, incorporated by reference in its entirety, and frozen and thawed multilamellar vesicles (FATMLV) as described above.

In one embodiment of the invention, the composition comprises lipid nanoparticles having a mean diameter of from about 20 nm to about 1000 nm, from about 50 nm to about 1000 nm, from 100 nm to about 1000 nm, from 200 nm to about 1000 nm, from 300 nm to about 1000 nm, from 400 nm to about 1000 nm, from 500 nm to about 1000 nm, from 600 nm to about 1000 nm, from 700 nm to about 1000 nm.

In a further embodiment, the mean diameter of the particles is from about 20 nm to about 2 μm, for example about 50 nm to about 1 μm, about 200 nm to about 1 μm, about 100 nm to about 800 nm, about 100 nm to about 600 nm or about 100 nm to about 500 nm.

The composition, in one embodiment comprises lipid particles with a mean diameter that is measured by a light scattering method, of approximately 0.005 microns to approximately 3.0 microns, for example, in the range about 0.1 μm to about 1.0 μm. In one embodiment, the mean diameter of the lipid particles in the composition is about 50 nm to about 2 μm, about 50 nm to about 1.5 μm, about 50 nm to about 1 μm, 50 nm to about 900 nm, about 50 nm to about 800 nm, about 50 nm to about 700 nm, about 50 nm to about 600 nm, about 50 nm to about 500 nm. In another embodiment, the mean diameter of the lipid particles in the composition is from about 200 nm to about 1.8 μm, from about 200 nm to about 1.7 μm, from about 200 nm to about 1.6 μm, from about 200 nm to about 1.5 μm, from about 200 nm to about 1.4 μm, from about 200 nm to about 1.3 μm, from about 200 nm to about 1.2 μm or from about 200 nm to about 1.1 μm.

In another embodiment, the composition comprises liposomes having a mean diameter of from about 20 nm to about 2 μm, from about 100 nm to about 2 μm, from about 100 nm to about 1.5 μm, from about 100 nm to about 1.3 μm, from about 100 nm to about 1.1 μm or from about 100 nm to about 900 nm.

The lipid particles, in one embodiment, comprise liposomes. In one embodiment, the liposomes have a mean diameter that is measured by a light scattering method, of approximately 0.01 microns to approximately 3.0 microns, for example, in the range about 0.2 to about 1.0 microns. In one embodiment, the mean diameter of the liposomes in the composition is about 150 nm to about 2 μm, about 200 nm to about 1.9 μm, about 200 nm to about 1.8 μm, about 200 nm to about 1.7 μm, about 200 nm to about 1.6 μm, about 200 nm to about 1.5 μm, about 200 nm to about 1.4 μm, about 200 nm to about 1.3 μm, about 200 nm to about 1.2 μm, about 200 nm to about 1.1 μm, about 200 nm to about 1 μm, 200 nm to about 900 nm, about 200 nm to about 800 nm, about 200 nm to about 700 nm, about 200 nm to about 600 nm, about 200 nm to about 500 nm.

In order to minimize dose volume and reduce patient dosing time, in one embodiment, it is important that liposomal entrapment or complexing of the lipid component to the disease-modifying antisarcoid compound be highly efficient and that the lipid-to disease-modifying antisarcoid compound ratio be at as low a value as possible. In one embodiment, the weight ratio of the lipid component to disease-modifying antisarcoid compound is 2 to 1 (“lipid component to disease-modifying antisarcoid compound” or “lipid component: disease-modifying antisarcoid compound”) or less (e.g., from about 2:1.0 to about 0.01:1.0, or from about 2:1.0 to about 0.1:1.0). In another embodiment, the weight ratio of the lipid component to disease-modifying antisarcoid compound is 1.5 to 1.0 (“lipid component to disease-modifying antisarcoid compound” or “lipid component:disease-modifying antisarcoid compound”) or less (e.g., from about 1.5:1.0 to about 0.01:1.0, or from about 1.5:1 to about 0.1:1.0). In another embodiment, the weight ratio of the lipid component to disease-modifying antisarcoid compound is 1.0 to 1.0 (“lipid component to disease-modifying antisarcoid compound” or “lipid component:disease-modifying antisarcoid compound”) or less (e.g., from about 1.0:1.0 to about 0.01:1.0, or from about 1.0:1.0 to about 0.1:1.0), or from about 1.0:1.0 to about 0.5:1.0.

In one embodiment, the pharmaceutical composition provided herein comprises at least one disease-modifying antisarcoid compound a phospholipid and a sterol (e.g., cholesterol). In a further embodiment, the pharmaceutical composition comprises a disease-modifying antisarcoid compound, DPPC and cholesterol.

The lipid complex, e.g., liposome, micelle, lipid microparticle, lipid nanoparticle in one embodiment, is further complexed to a targeting moiety. The targeting moiety is a moiety that targets a specific cell type, for example a mononuclear phagocyte such as a monocyte or macrophage. The targeting moiety in one embodiment is an antibody or antigen binding portion thereof, a lectin, a peptide or an additional anionic lipid complexed to the surface of the lipid complex. Various targeting moieties are provided in Kelly et al. (2011). Journal of Drug Delivery 2011, Article 727241, doi: 10.1155/2011/727241, the contents of which are incorporated by reference herein in its entirety, see for example Table 1 of Kelly. Peptides such as muramyl tripeptide, ARG-Gly-Asp, antibodies or antigen binding portions of anti-VCAM-1, anti-CC52, anti-CC531, anti-CD11c/DEC-205, lectins such as Mann-C4-chol, Man₂DOG, aminophenyl-α-D-mannopyranoside, Man3-DPPE are all amenable for use with the lipid complexes provided herein. Other ligands such as maleylated bovine serum albumin (MBSA), O-steroly amylopectin (O-SAP), fibronectin and galactosyl can also be employed at the surface of a lipid complex to target a mononuclear phagocyte.

As described above, the composition in one embodiment includes lipid microparticles, lipid nanoparticles, liposomes or a combination thereof. The composition in one embodiment comprises lipid microparticles or nanoparticles comprising one or more of the disease-modifying antisarcoid compounds as described herein complexed to a lipid component, and a hydrophobic additive. In one embodiment, the hydrophobic additive (e.g., an additive that is at least partially hydrophobic) is a hydrocarbon, a terpene compound or a hydrophobic lipid (e.g., tocopherol, tocopherol acetate, sterol, sterol ester, alkyl ester, vitamin A acetate, a triglyceride, a phospholipid). The hydrocarbon can be aromatic, an alkane, alkene, cycloalkane or an alkyne. In one embodiment, the hydrocarbon is an alkane (i.e., a saturated hydrocarbon). In another embodiment, the hydrocarbon is a C₁₅-C₅₀ hydrocarbon. In a further embodiment, the hydrocarbon is a C₁₅, C₂₀, C₂₅, C₃₀, C₃₅, C₄₀, C₄₅ or C₅₀ hydrocarbon. In yet another embodiment, the hydrophobic additive is a C₁₅-C₂₅ hydrocarbon, C₁₅-C₃₅ hydrocarbon, C₁₅-C₄₅ hydrocarbon, C₁₅-C₂₀ hydrocarbon, C₂₀-C₂₅ hydrocarbon, C₂₅-C₃₀ hydrocarbon, C₃₀-C₃₅ hydrocarbon, C₃₅-C₄₀ hydrocarbon, C₄₀-C₄₅ hydrocarbon or a C₄₅-C₅₀ hydrocarbon.

The hydrophobic additive, when present in the composition, in one embodiment, is present at 25 mol %-50 mol %, for example, 30 mol %-50 mol %, 35 mol %-45 mol %. In even a further embodiment, the hydrophobic additive is present in the composition at about 40 mol % or about 45 mol %.

In one embodiment, a composition comprising a disease-modifying antisarcoid compound, a lipid component, and a terpene compound (e.g., the hydrophobic additive) is provided. The composition, in a further embodiment, comprises a cationic lipid, e.g., a PEGylated cationic lipid, as the lipid component. The terpene compound (hydrophobic additive), in one embodiment, is a hydrocarbon (e.g., isoprene, squalane or squalene). In another embodiment, the terpene compound is a hemiterpene (C₅H₈), monoterpene (C₁₀H₁₆), sesquiterpene (C₁₅H₂₄), diterpene (C₂₀H₃₂) (e.g., cafestol, kahweol, cembrene, taxadiene), sesterterpene (C₂₅H₄₀), triterpene (C₃₀H₄₈), sesquaterpene (C₃₅H₅₆), tetraterpene (C₄₀H₆₄), polyterpene (e.g., a polyisoprene with trans double bonds) or a norisoprenoid (e.g., 3-oxo-α-ionol, 7,8-dihydroionone derivatives). The terpene compound, in another embodiment, is selected from one of the compounds provided in Table 2, below. In one embodiment, the hydrophobic additive is squalane.

The composition provided herein, in one embodiment, comprises an antisarcoid compound and one or more PEGylated lipids. In a further embodiment, the composition comprises a hydrophobic additive, as described above. In one embodiment, the composition provided herein comprises an antisarcoid compound, a hydrophobic additive and a PEGylated lipid. In a further embodiment, the hydrophobic additive comprises a hydrocarbon e.g, a terpene compound.

TABLE 2
Terpene hydrophobic additives amenable for use in the compositions of the present invention.

| Name | Formula |
|---|---|
| Isoprene | 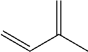 |
| Limonene | 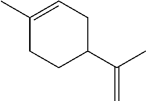 |
| humulene | 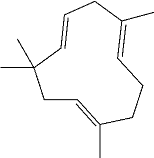 |
| farnasene | 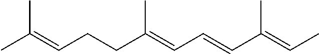 |
| squalene | 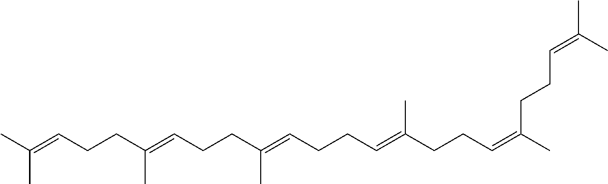 [SMILES: CC(C)=CCC/C(C)=C\CC/C(C)=C/CC/C=C(\C)CC/C=C(\C)CCC=C(C)C] |
| squalane | 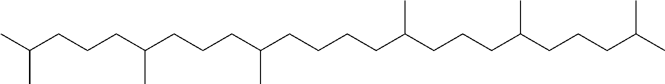 [SMILES: CC(C)CCCC(C)CCCC(C)CCCCC(C)CCCC(C)CCCC(C)C] |

Yet another aspect of the invention is directed to a kit comprising a composition comprising a pharmaceutically effective amount of the compound of Formula I, or pharmaceutically acceptable salt of the compound of Formula I, and a pharmaceutically acceptable excipient, and an inhalation delivery device.

The devices and/or compositions described here may be packaged and/or distributed (e.g., to hospitals, clinics, physicians, and/or patients) in an administration kit. Such kits may comprise one or more inhalation devices (e.g., MDI, DPI or nebulizer), and one or more containers (e.g., unit doses or multi-dose containers) of the composition. In one embodiment, the inhalation delivery device is a dry powder inhaler (DPI), metered dose inhaler (MDI), soft mist inhaler, or a nebulizer. In some variations, the kit may include one or more devices that are already loaded with the composition. For example, a device may comprise a reservoir that is pre-filled with the composition. Certain variations of kits may include multiple different compositions, and/or multiple different dosages of the same composition. The kit may additionally comprise a carrier or diluent, a case, and/or instructions for operating the appropriate device.

### EXAMPLES

The present invention is further illustrated by reference to the following Examples. However, it is noted that these Examples, like the embodiments described above, are illustrative and are not to be construed as restricting the scope of the invention in any way.

“EDC” means N-(3-dimethylaminopropyl)-N′-ethylcarbodiimide hydrochloride. “DCC” means N,N′-dicyclohexylcarbodiimide. “DIC” means N,N′-diisopropylcarbodiimide. “PyBOP” means (benzotriazol-1-yloxy)tripyrrolidinophosphonium hexafluorophosphate. “HATU” means 1-[bis(dimethylamino)methylene]-1H-1,2,3-triazolo[4,5-b]pyridinium 3-oxid hexafluorophosphate. “HOBt” means 1-hydroxybenzotriazole. “HOAt” means 1-hydroxy-7-azabenzotriazole.

### Example 1

### Synthesis of the Compounds of Formula (I)

The compounds of Formula (I) may be prepared according to methods known to those skilled in the art. The following examples disclose methods suitable for preparing compounds of Formula (Ia), (Ib), (Ic), (Id), (Ie), (If) and (Ig).

### Example 1(a)

### Synthesis of Compounds of Formula (Ia)

### Esterfication of MPA Via an Acid Chloride:

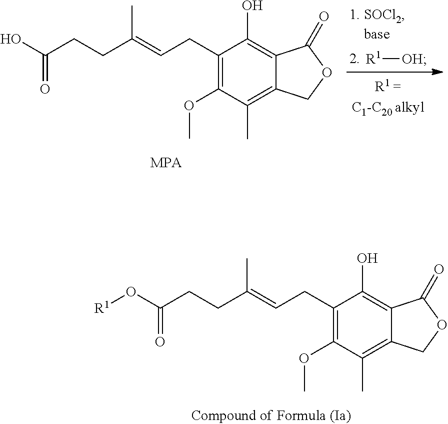

Compounds of Formula (Ia) may be prepared using esterification methods that are known to those skilled in the art. For example, in a first step, mycophenolic acid (MPA) may be converted to the corresponding acid chloride by treatment with thionyl chloride (SOCl₂) and a base, such as trimethylamine (TEA). In a second step, the resulting acid chloride so-formed may be reacted with a C₁-C₂₀ alkyl alcohol to provide the compound of Formula (Ia).

### Alkylation of MPA:

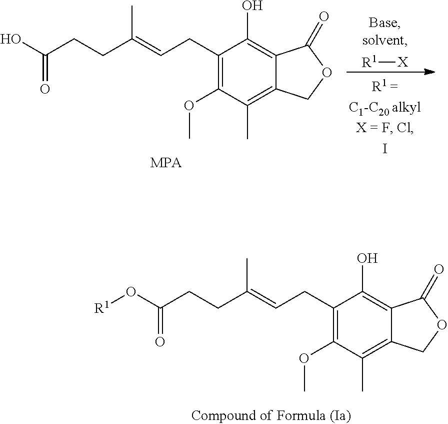

Compounds of Formula (Ia) may be prepared by alkylation methods known to those skilled in the art. For example, MPA may be treated with a C₁-C₂₀ alkyl halide in a suitable solvent (such as dimethylformamide) in the presence of a base to provide the compound of Formula (Ia).

### Alkylation of MPS:

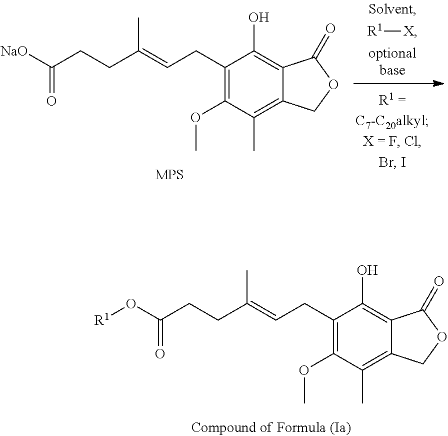

In another example, compounds of Formula (Ia) may be prepared by the direct alkylation of sodium mycophenolate (MPS) with an C₁-C₂₀ alkyl halide in a suitable solvent (such as dimethylformamide), optionally in the presence of a base to provide the compound of Formula (Ia).

### Esterification of MPA Using an Ester Coupling Reagent:

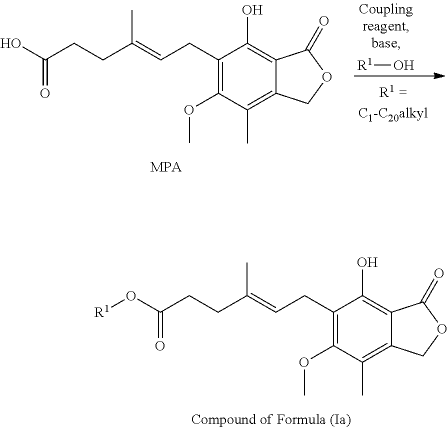

In another example, mycophenolic acid (or a salt thereof) may be treated with a suitable ester coupling reagent and, optionally, a suitable ester coupling additive in the presence of a C₁-C₂₀ alkyl alcohol to provide a compound of Formula (Ia). Suitable ester coupling agents are known to those skilled in the art and include EDC, DCC, DIC, PyBOP, HATU, etc. Suitable ester coupling additives are known to those skilled in the art and include HOBt, HOAt, etc.

### Example 1(b)

### Synthesis of Compounds of Formula (Ib)

Synthesis of Compounds of Formula (Ib), Wherein R¹ is C₁-C₂₀ Alkyl:

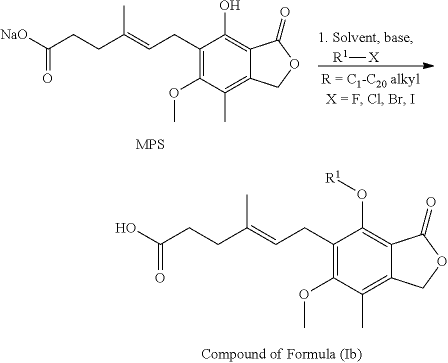

Compounds of Formula (Ib), wherein R¹ is C₁-C₂₀ alkyl, may be prepared by etherfication methods known to those skilled in the art. For example, MPS may be treated with a C₁-C₂₀ alkyl halide in a suitable solvent (such as dimethylformamide) in the presence of a base to provide the compound of Formula (Ib).

Synthesis of Compounds of Formula (Ib), Wherein R¹ is C(O)C₁-C₁₉ Alkyl:

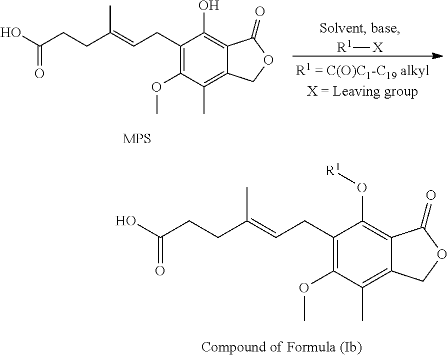

Compounds of Formula (Ib), wherein R¹ is C(O)C₁-C₁₉ alkyl, may be prepared by esterification methods known to those skilled in the art. For example, MPA may be treated with an activated carboxylic acid derivative of formula X—C(O)C₁-C₁₉ alkyl, wherein X is a leaving group, in a suitable solvent and in the presence of a base. Activated carboxylic acid derivatives are known in the art and include, for example, acid anhydrides, acid halides, etc. or carboxylic acid salts in the presence of a suitable ester coupling agent and suitable ester coupling additive.

### Example 1(c)

### Synthesis of Compounds of Formula (Ic)

Synthesis of Compounds of Formula (Ic), Wherein R² is C(O)C₁-C₁₉ Alkyl:

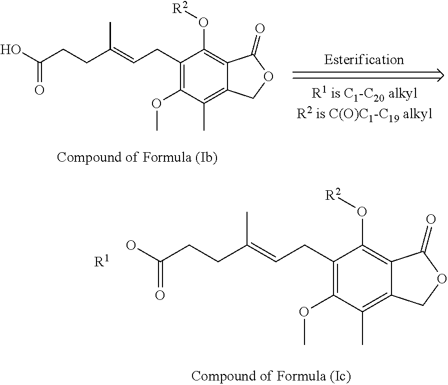

In one example, compounds of Formula (Ic), wherein R² is C(O)C₁-C₁₉ alkyl, may be prepared by esterification of the carboxylic acid group present in the compounds of Formula (Ib). Such esterification methods are known to those skilled in the art.

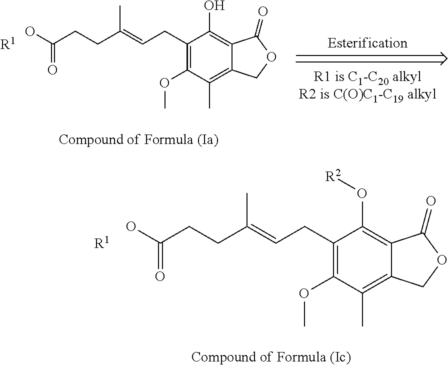

In one example, compounds of Formula (Ic), wherein R² is C(O)C₁-C₁₉ alkyl, may be prepared by esterification of the phenol group present in the compounds of Formula (Ia). Such esterification methods are known to those skilled in the art.

Synthesis of Compounds of Formula (Ic), Wherein R² is C₁-C₂₀ Alkyl:

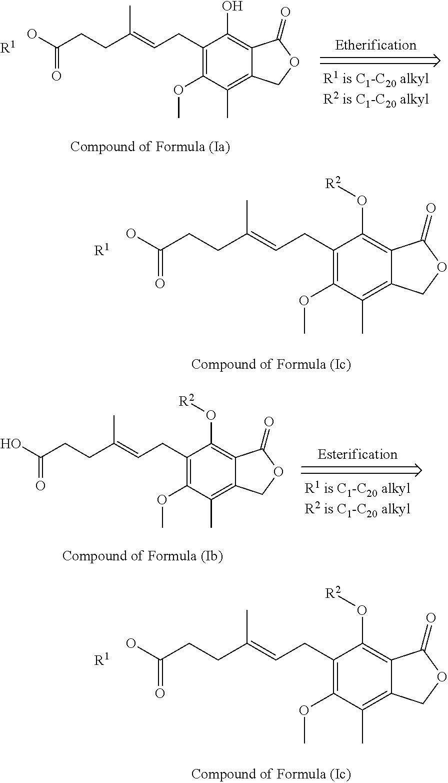

Compounds of Formula (Ic), wherein R² is C₁-C₂₀ alkyl, may be prepared either by esterification of a compound of Formula (Ia), wherein R¹ is C₁-C₂₀ alkyl, or by etherification of a compound of Formula (Ib), wherein R² is s C₁-C₂₀ alkyl. Such esterification and etherfication may be conducted using methods known to those skilled in the art.

### Example 1(d)

### Synthesis of Compounds of Formula (Id)

### Amidation of MPA Via an Acid Chloride:

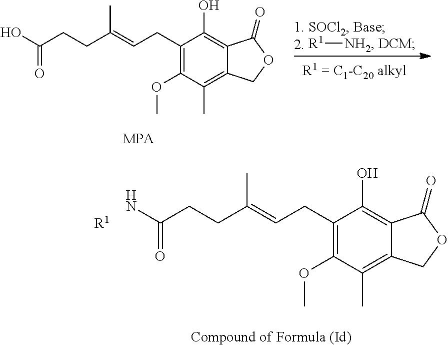

Compounds of Formula (Id) may be prepared using amide-forming methods known to those skilled in the art. For example, in a first step, MPA may be converted to the corresponding acid chloride by treatment with thionyl chloride and a base, such as triethylamine. In a second step, the acid chloride so-formed may be reacted with an C₁-C₂₀ alkyl amine to provide a compound of Formula (Id).

### Amidation of MPS Using an Amide Coupling Reagent:

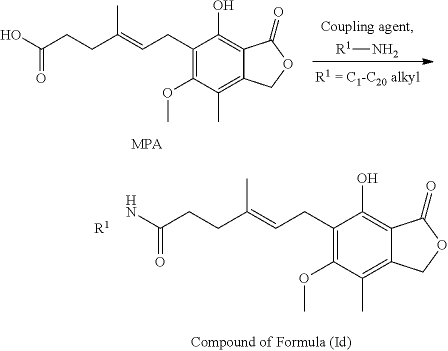

Compounds of Formula (Id) may be prepared using direct amide-forming methods known to those skilled in the art. For example, MPA (or a suitable salt thereof) may be treated with a suitable amide coupling reagent and, optionally, a suitable amide coupling additives in the presence of a C₁-C₂₀ alkyl amine to provide a compound of Formula (Id). Suitable amide coupling reagents are known in the art and include EDC, DCC, DIC, PyBOP, HATU, etc. Suitable amide coupling additives are known in the art and include HOBt, HOAt, etc.

### Example 1(e)

### Synthesis of Compounds of Formula (Ie)

Synthesis of Compounds of Formula (Ie), Wherein R² is C(O)C₁-C₁₉ Alkyl:

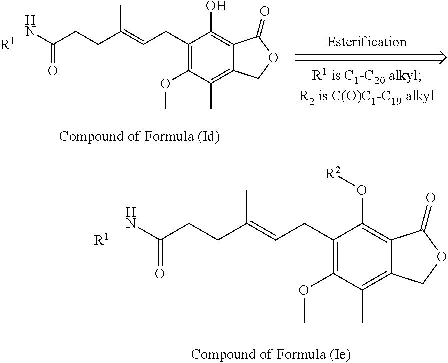

Compounds of Formula (Ie), wherein R² is C(O)C₁-C₁₉ alkyl, may be prepared by esterification of the phenol group present in a compound of Formula (Id). For example, Formula (Id) may be treated with an activated carboxylic acid derivative of formula X—C(O)C₁-C₁₉ alkyl, wherein X is a leaving group, in a suitable solvent and in the presence of a base. Activated carboxylic acid derivatives are known in the art and include acid anhydrides, acyl chlorides, etc. or carboxylic acid salts in the presence of a suitable ester coupling agent and suitable ester coupling additive.

Synthesis of Compounds of Formula (Ie), Wherein R² is C₁-C₂₀ Alkyl:

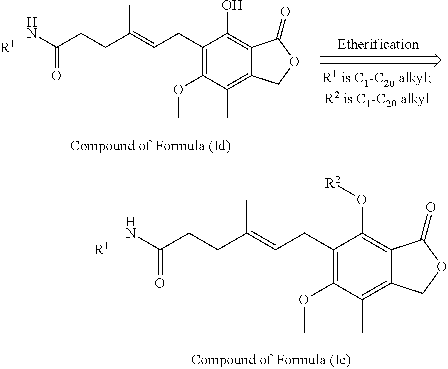

In one example, compounds of Formula (Ie), wherein R² is C₁-C₂₀ alkyl, may be prepared by alkylation of the phenol group present in the compound of Formula (Id). Such alkylation methods are known to those skilled in the art and include treating a compound of Formula (Id) with a C₁-C₂₀ alkyl halide and base.

### Example 1(f)

### Synthesis of Compounds of Formula (If)

### Thioesterification of MPA Using an Ester Coupling Reagent:

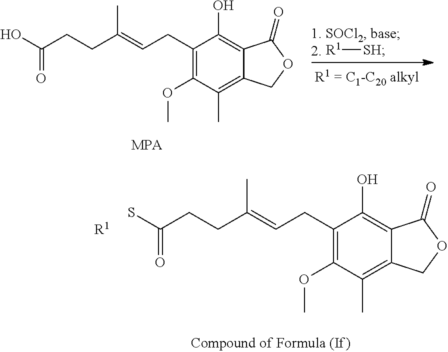

Compounds of Formula (If) may be prepared using thioesterification methods known to those skilled in the art. For example, MPA may be converted to the corresponding acid chloride by treatment with thionyl chloride and a base, such as triethylamine. The activated acid chloride may then be react with a C₁-C₂₀ alkyl thiol to provide a compound of Formula (If).

### Thioesterification of MPA Using an Ester Coupling Reagent:

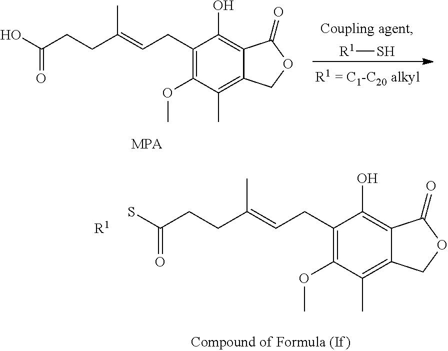

In another example, MPA (or a suitable salt thereof) may be treated with a suitable ester coupling reagents and, optionally, a suitable ester coupling additive in the presence of a C₁-C₂₀ alkyl thio to provide a compound of Formula (If). Suitable ester coupling agents are known to those skilled in the art and include EDC, DCC, DIC, PyBOP, HATU, etc. Suitable ester coupling additives are known to those skilled in the art and include HOBt, HOAt, etc.

### Example 1(g)

### Synthesis of Compounds of Formula (Ig)

Synthesis of Compounds of Formula (Ig), Wherein R² is C(O)C₁-C₁₉ Alkyl:

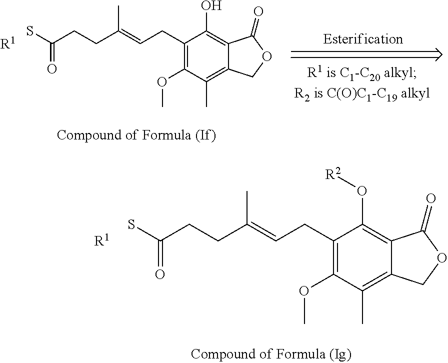

Compounds of Formula (Ig), wherein R² is C(O)C₁-C₁₉ alkyl, may be prepared by esterification of the phenol group present in a compound of Formula (If) using methods known to those skilled in the art. For example, a compound of Formula (If) may be treated with an activated carboxylic acid derivative of formula X—C(O)C₁-C₁₉ alkyl, wherein X is a leaving group, in a suitable solvent and in the presence of a base. Activated carboxylic acid derivatives are known in the art and include acid anhydrides, acyl chlorides, etc. or carboxylic acid salts in the presence of a suitable ester coupling agent and suitable ester coupling additive.

Synthesis of Compounds of Formula (Ig), Wherein R² is C₁-C₂₀ Alkyl:

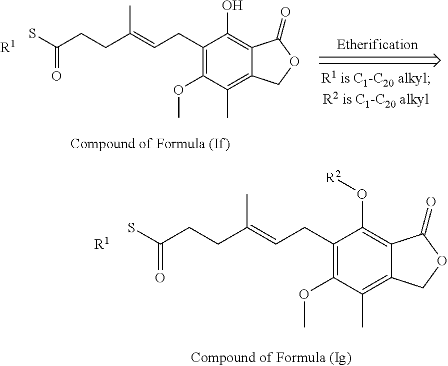

Compounds of Formula (Ig), wherein R² is C₁-C₂₀ alkyl, may be prepared by alkylation of the phenol group present in a compound of Formula (If) using etherfication methods known to those skilled in the art. For example, a compound of Formula (If) may be treated with a C₁-C₂₀ alkyl halide and base to provide a compound of Formula (Ig).

### Example 1(h)

### Synthesis of Mono-C₁₂MP and Bis-C₁₂MP

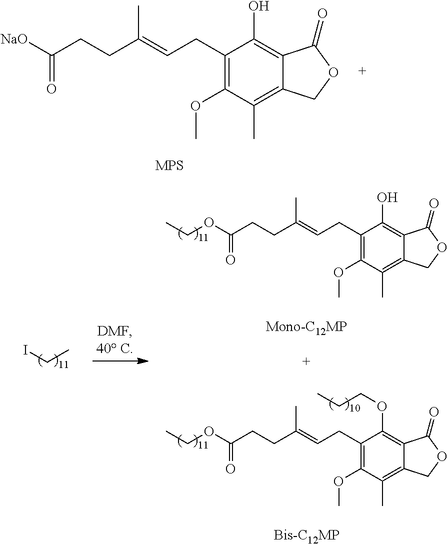

To a 100 mL round bottom flask equipped with a stir bar was added MPS (250.1 mg, 0.73 mmol) and DMF (20 mL). The reaction mixture was heated to 40° C. to fully dissolve the solid MPS, at and a single aliquot of 1-iodododecane (180.3 μL, 0.73 mmol, 1 eq.) was added to the reaction mixture. The reaction mixture was allowed to stir at 40° C. for three hours. Solvent was removed under reduced pressure to yield a yellow solid that was dissolved in ethyl acetate (50 mL) and washed with DI H2O (3×25 mL) and 0.01M NaOH (2×25 mL) The organic layer was dried over anhydrous Na₂SO₄, filtered, and evaporated to dryness. The crude material was purified using preparatory HPLC (100×21.2 mm ACE CN 5 μm column) to isolate Mono-C12MP as a white flaky solid and Bis-C12MP as a thick off-white oil.

Mono-C₁₂MP: ¹H NMR (500 MHz, CDCl₃) δ=0.88 (t, J=8 Hz, 3H), 1.25-1.30 (m, 18H), 1.54-1.60 (m, 2H), 1.80 (s, 3H), 2.15 (s, 3H), 2.25-2.33 (m, 2H), 2.36-2.40 (m, 2H), 3.39 (d, J=7 Hz, 2H), 3.76 (s, 3H), 4.01 (t, J=8 Hz, 2H), 5.20 (s, 2H), 5.24 (t, J=8 Hz, 1H), 7.67 (s, 1H) ppm; HRMS (ESI, 2:2:1 MeCN:MeOH:H₂O): m/z=489.3204 ([M+H]⁺).

Bis-C₁₂MP: ¹H NMR (500 MHz, CDCl₃) δ=0.88 (t, J=8 Hz, 6H), 1.25-1.34 (m, 36H), 1.41-1.47 (m, 2H), 1.54-1.60 (m, 2H), 1.79 s, 3H), 1.82-1.84 (m, 2H), 2.17 (s, 3H), 2.27-2.30 (m, 2H), 2.36-2.39 (m, 2H), 3.41 (d, J=7 Hz, 2H), 3.78 (s, 3H), 4.00 (t, J=7 Hz, 2H), 4.20 (t, J=7 Hz, 2H), 5.11 (s, 2H), 5.18 (t, J=7 Hz, 1H) ppm, HRMS (ESI, 2:2:1 MeCN:MeOH:H₂O): m/z=657.5081 ([M+H]⁺).

### Example 1(j)

### Synthesis of Mono-C₁₆MP and Bis-C₁₆MP

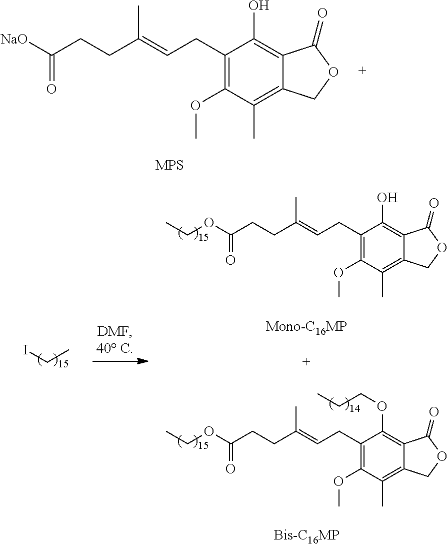

To a 100 mL round bottom flask equipped with a stir bar was added MPS (276.4 mg, 0.80 mmol) and DMF (25 mL). The reaction mixture was heated to 40° C. to fully dissolve the MPS and a single aliquot of 1-iodohexadecane (507.8 μL, 1.61 mmol, 2 eq) was added. The reaction mixture was allowed to stir at 40° C. for two hours at which point solvent was removed under reduced pressure to yield a yellow solid. The crude material was dissolved in ethyl acetate (200 mL) and washed with deionized H₂O (2×100 mL) and 0.01 M NaOH (2×100 mL). The organic layer was dried over anhydrous Na₂SO₄, filtered, and evaporated to dryness. The crude material was purified using preparatory HPLC (250×10.0 mm ACE C18 5 μm column) to isolate Mono-C₁₆MP as a white flaky solid and Bis-C₁₆MP as a thick off-white oil.

Mono-C₁₆MP: ¹H NMR (500 MHz, CDCl₃) δ=0.88 (t, J=8 Hz, 3H), 1.25-1.31 (m, 26H), 1.54-1.60 (m, 2H), 1.80 (s, 3H), 2.15 (s, 3H), 2.25-2.31 (m, 2H), 2.37-2.40 (m, 2H), 3.39 (d, J=7 Hz, 2H), 3.76 (s, 3H), 4.00 (t, J=8 Hz, 2H), 5.20 (s, 2H), 5.24 (t, J=8 Hz, 1H), 7.67 (s, 1H); ¹³C NMR (126 MHz, CDCl₃) δ=11.1, 13.6, 15.6, 22.1, 22.2, 25.4, 28.1, 28.8, 28.9, 29.0, 29.1, 29.2, 31.4, 32.6, 34.2, 60.5, 64.0, 69.5, 105.9, 116.2, 121.7, 122.1, 133.8, 143.5, 153.1669, 163.2, 172.4, 172.9 ppm. HRMS (ESI, 2:2:1 MeCN:MeOH:H₂O): m/z=545.3828 ([M+H]⁺).

Bis-C₁₆MP: ¹H NMR (500 MHz, CDCl₃) δ=0.88 (t, J=7 Hz, 6H), 1.26-1.33 (m, 54H), 1.41-1.47 (m, 2H), 1.55-1.58 (m, 2H), 1.79 (s, 3H), 1.82-1.84 (m, 2H), 2.17 (s, 3H), 2.27-2.32 (m, 2H), 2.36-2.39 (m, 2H), 3.41 (d, J=7 Hz, 2H), 3.76 (s, 3H), 4.00 (t, J=7 Hz, 2H), 4.20 (t, J=7 Hz, 2H), 5.11 (s, 2H), 5.18 (t, J=7 Hz, 1H) ppm. HRMS (ESI, 2:2:1 MeCN:MeOH:H₂O): m/z=769.6336 ([M+H]⁺).

### Example 2

### Preparation of Micelle Compositions

To test the uptake and activity of the compounds of Formula (I) complexed with or encapsulated by lipid components, formulations 1-5 were prepared. These formulations are summarized in Table 3.

Micellar formulations were prepared by rapidly injecting acetone solutions of methoxypolyethylene glycol PEG 2000 (DMG-PEG2000) and a compound of the invention into phosphate buffered saline (PBS) with vortexing. The micelle dispersions were washed by tangential flow filtration with 5 volumes of PBS to remove the organic solvent. Finally, the formulations were filtered through 0.2 μm PVDF syringe filters providing the compositions listed in Table 3. The relative concentration of each component listed in Table 3 is nominal, since PEG2000 was not measured after processing.

TABLE 3
Summary of micelle formulation

| | DMG- | | | |
|---|---|---|---|---|
| | PEG2000 | Prodrug | Peak Diameter | Polydispersity |
| | (molar %) | (molar %) | (nm) | (nm) |
| Control | 100 | — | 12.5 | 1.4 |
| m-C₁₂MP | 90 | 10 | 11.6 | 0.2 |
| bis-C₁₂MP | 90 | 10 | 14.7 | 3.9 |
| m-C₁₆MP | 40 | 60 | 26.5 | 13.1 |
| m-C₁₆MP | 90 | 10 | 15.0 | 2.4 |

m-C₁₂MP: dodecyl mycophenolate, or a compound of Formula (Ia) wherein R¹ is C₁₂ alkyl and R² is hydrogen; bis-C₁₂MP: bis-dodecyl mycophenolate, or a compound of Formula (Ic) wherein R¹ and R² are C₁₂ alkyl; m-C₁₆MP: hexadecyl mycophenolate, or a compound of Formula (Ia) wherein R¹ is C₁₆ alkyl and R² is hydrogen; bis-C₁₆MP: bis-hexadecyl mycophenolate, or a compound of Formula (Ic) wherein R¹ and R² are C₁₆ alkyl; DMG- PEG2000: methoxypolyethylene glycol PEG 2000.

### Example 3

### Preparation of Liposome Compositions

To test the uptake and activity of the compounds of Formula (I) complexed with or encapsulated by liposome compositions, formulations 5-11 were prepared. These formulations are summarized in Table 4.

Liposomal formulations were prepared by rapidly injecting organic solutions of the lipids and a compound of the invention into phosphate buffered saline (PBS) with vortexing. The liposome dispersions were washed by tangential flow filtration with 5 volumes of PBS to remove the organic solvent. Finally, the liposomes were filtered through 0.2 μm PVDF syringe filters providing the compositions listed in Table 4.

TABLE 4
Summary of liposome formulations:

| | | | | | | Average | |
|---|---|---|---|---|---|---|---|
| | DPPC | DPPG | Cholesterol | CHEMS | Prodrug | Diameter | Polydispersity |
| | (molar %) | (molar %) | (molar %) | (molar %) | (molar %) | (nm) | (%) |
| Control | 63 | 5 | 32 | — | — | 116.4 | 18.7 |
| m-C₁₂MP | 38 | 3 | 18 | — | 41 | 143.5 | 19.0 |
| bis-C₁₂MP | 48 | 3 | 17 | — | 32 | 111.9 | 10.4 |
| m-C₁₆MP | 45 | 4 | 24 | — | 27 | 120.7 | 9.0 |
| bis-C₁₆MP | 41 | 3 | 21 | — | 35 | 171.0 | 5.8 |
| Control | 73 | — | — | 27 | — | 131.8 | 13.8 |
| m-C₁₆MP | 48 | — | — | 16 | 36 | 90.2 | 15.8 |

m-C₁₂MP: dodecyl mycophenolate, or a compound of Formula (Ia) wherein R¹ is C₁₂ alkyl and R² is hydrogen; bis-C₁₂MP: bis-dodecyl mycophenolate, or a compound of Formula (Ic) wherein R¹ and R² are C₁₂ alkyl, m-C₁₆MP: hexadecyl mycophenolate, or a compound of Formula (Ia) wherein R¹ is C₁₆ alkyl and R₂ is hydrogen; bis-C₁₆MP: bis-hexadecyl mycophenolate, or a compound of Formula (Ic) wherein R¹ and R² are C₁₆ alkyl; DPPC: dipalmitoylphosphatidylcholine; DPPG: dipalmitoylphosphatidylglycerol; CHEMS: cholesterol hemisuccinate.

dipalmitoylphosphatidylcholine; DPPG: dipalmitoylphosphatidylglycerol; CHEMS: cholesterol hemi succinate.

### Example 4

### Inhibition of TNF-α Production in Rat and Human Macrophages

TNF-α is pro-inflammatory cytokine involved in systemic inflammation and contributes to the acute phase of immune response. Although many cells produce TNF-α, macrophages are the major producers of TNF-α and are also highly responsive to TNF-α. Dysregulation of TNF-α production is associated with a variety of human diseases. TNF-α promotes the inflammatory response and in turn causes pathogenesis associated with inflammation.

The ability of the mycophenolate sodium to decrease lipopolysaccharide (LPS)-stimulated TNF-α production in macrophages was studied.

### Rat Macrophages:

Rat alveolar macrophages (NR8383) were co-treated with 25 ng/mL lipopolysaccharide (LPS) and mycophenolate sodium at concentrations ranging from 0.15 to about 15 μm for a period of 20 h. After this period, the cell supernatants were collected for TNF measurement and the remaining macrophages were assessed for cytotoxicity. TNF concentrations were assayed using a rat TNF ELISA kit available from ThermoFisher. Cytotoxicity was determined using the CellTox green assay available from Promega.

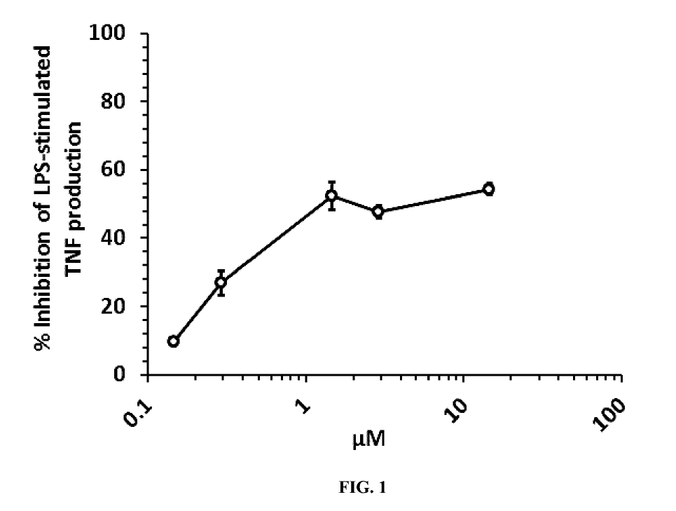

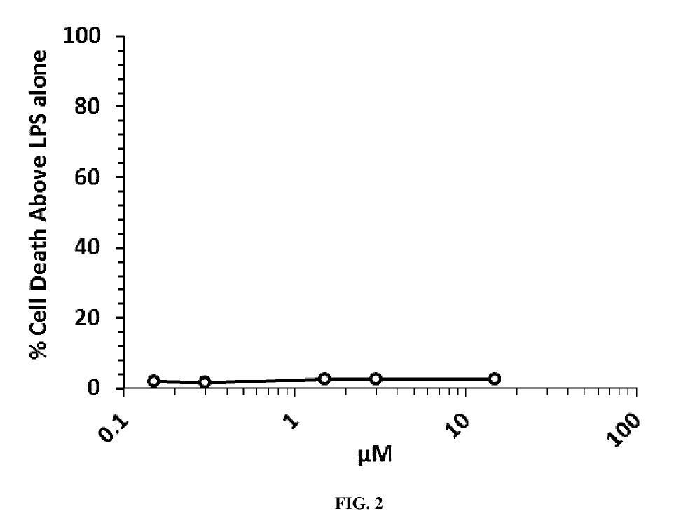

Inhibition of TNF-α production was calculated as a percentage of LPS-stimulated TNF production in the absence of mycophenolate sodium. The calculated IC₅₀ was 1.5 μM and the Eₘₐₓ, was 54%. As shown in FIG. 1, a mycophenolate sodium concentration dependent reduction in TNF production was observed. As shown in FIG. 2, there was no significant cytotoxicity at any of the mycophenolate concentrations tested.

### Human Macrophages:

THP-1 cells were cultured in media with 50 ng/mL phorbol myristate acetate (PMA) for 24 h and then in fresh media without PMA for an additional 24 h. The PMA treatment resulted in THP-1 derived human macrophages, which were seeded into 96-well plates. The well plates were treated with mycophenolate sodium at concentrations ranging from 0.03 to 10 μm and incubated for a period for 4 h. After this period, LPS was added to each well plate at a concentration of 100 pg/mL and the LPS-, mycophenolate-treated well plates were incubated for 16 h. After 16 h, the cell supernatants were collected for TNF measurement and the remaining macrophages were assessed for cytotoxicity. TNF concentrations were assayed using a human TNF ELISA kit available from ThermoFisher. Cytotoxicity was determined using the CellTox green assay available from Promega.

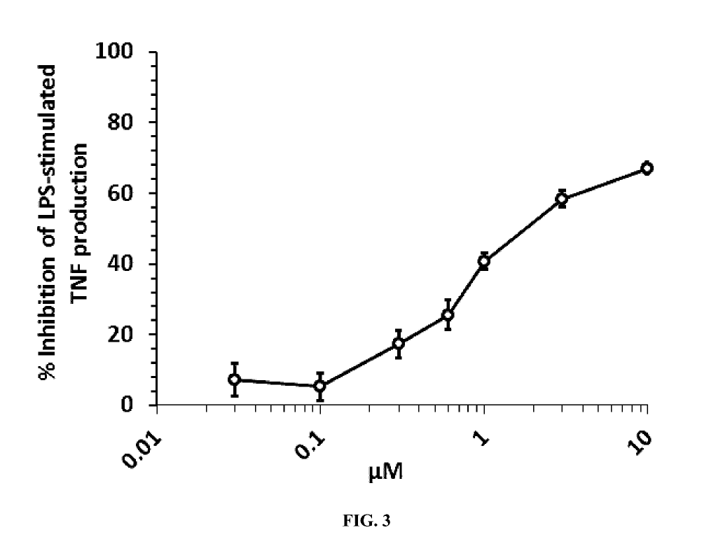

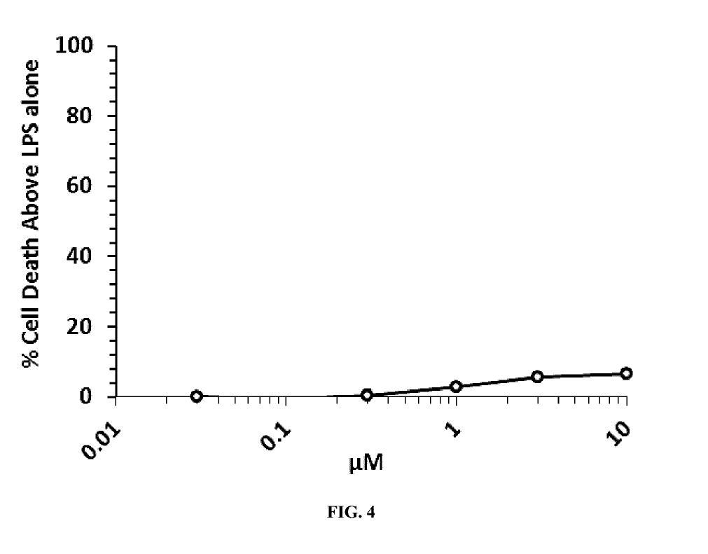

Inhibition of TNF-α production was calculated as a percentage of LPS-stimulated TNF production in the absence of mycophenolate sodium. The calculated IC₅₀ was 1.4 μM and the Eₘₐₓ was 67%. As shown in FIG. 3, a mycophenolate sodium concentration dependent reduction in TNF production was observed. As shown in FIG. 4, there was no significant cytotoxicity at mycophenolate sodium concentrations<3 μM, but mild cytotoxicity (P<0.05) of 6.6 and 8.9% was observed at 3 and 10 μM mycophenolate sodium, respectively.

Using the above-described methods, the ability of the compounds of the invention to decrease lipopolysaccharide (LPS)-stimulated TNF-α production in macrophages can be tested.

### Example 5

### Lung to Plasma Exposure Ratio for Inhaled Dosing of MPS and MMF

Studies to determine the lung to plasma ratio obtained by inhalation of mycophenolate sodium (MPS) and mycophenolate mofetil (MMF), as well as peroral dosing of MMF were performed.

In the inhalation studies, C57BL/6 mice were given MPS, or MMF, by inhalation and the lung and plasma mycophenolic acid (MPA) concentrations were measured at t=0, 0.5, 1, 2, 4, and 6 h post-dose.

In the MMF peroral study, C57BL/6 mice were given MMF by oral gavage and the lung and plasma mycophenolic acid (MPA) concentrations were measured at t=0, 0.5, 1, 2, 4, and 6 h post-dose.

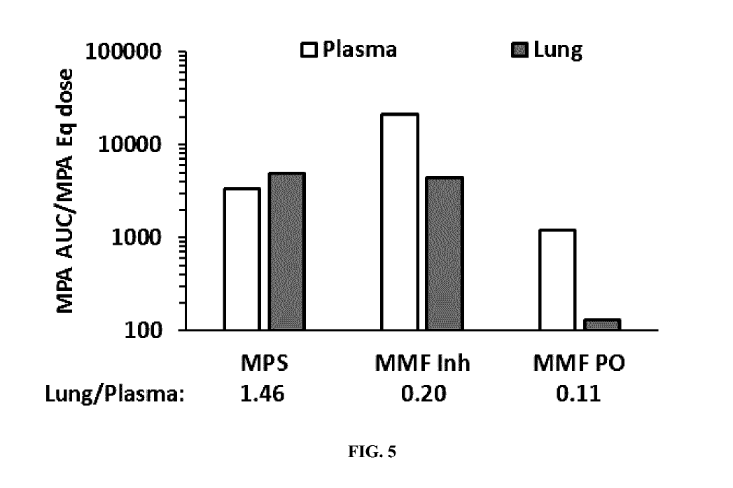

For each study, the mean concentrations at each timepoint were used to calculate the AUC₀₋₆, which was normalized to the MPA-equivalent dose administered to each group. As shown in FIG. 5, the ratio of lung/plasma AUC₀₋₆ for inhaled MPS was 1.46; inhaled MMF was 0.20 and peroral MMF was 0.11.

Using the above-described methods, the lung to plasma ratio obtained by inhalation of the compounds of the invention can be tested.

### Example 6

### Comparison of Lung to Plasma Exposure Ratio for Inhaled Dosing of Mono-C₁₂-MP MPS, MMF

Studies to determine the lung to plasma ratio obtained by inhalation of mycophenolate sodium (MPS), mycophenolate mofetil (MMF), and hexadecyl mycophenolate (mono-C₁₆MP) were conducted.

In each study group, C57BL/6 mice were given MPS, MMF or mono-C₁₆MP by inhalation and the lung and plasma mycophenolic acid (MPA) concentrations were measured at t=0, 0.5, 1, 2, 4, and 6 h post-dose.

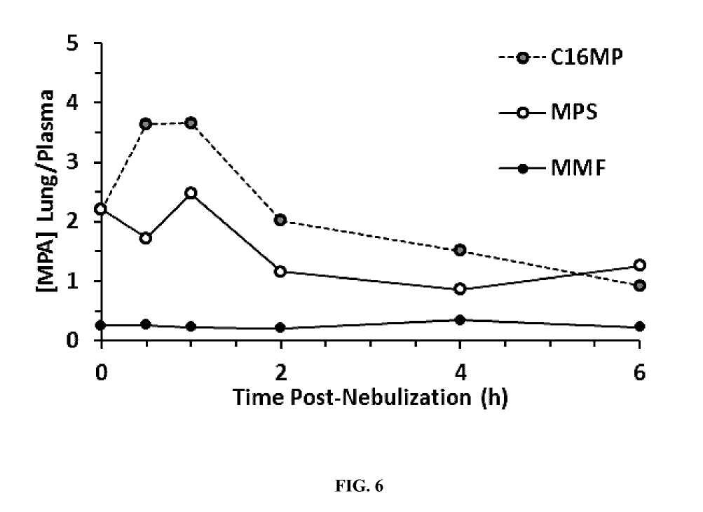

For each mouse in the study, the lung to plasma MPA ratios were calculated; the individual calculated ratios were averaged for each treatment group and the average values were plotted, shown in FIG. 6. The data indicate that mono-C₁₆MP exhibits a higher lung to plasma exposure ratio of MPA after inhalation compared to the ratio obtained with MPS or MMF.

### Example 7

### Inhibition of TNF-α Production in the Lungs of C57BL/6 Mice by MPS

The ability of the mycophenolate sodium to decrease lipopolysaccharide (LPS)-stimulated TNF-α production in the lungs of C57BL/6 mice was studied.

C57/BL6 mice were dosed with mycophenolate sodium (MPS) by inhalation and then given 1 mg/kg LPS by intratracheal instillation. In a control experiment, C57/BL6 mice were dosed with phosphate-buffered saline (PBS) by inhalation and then given 1 mg/kg LPS by intratracheal instillation. Bronchoalveolar lavage fluid was collected 4 h after the intratracheal instillation of LPS. The TNF concentrations from the lavage fluid were measured using a mouse TNF ELISA kit available from Invitrogen.

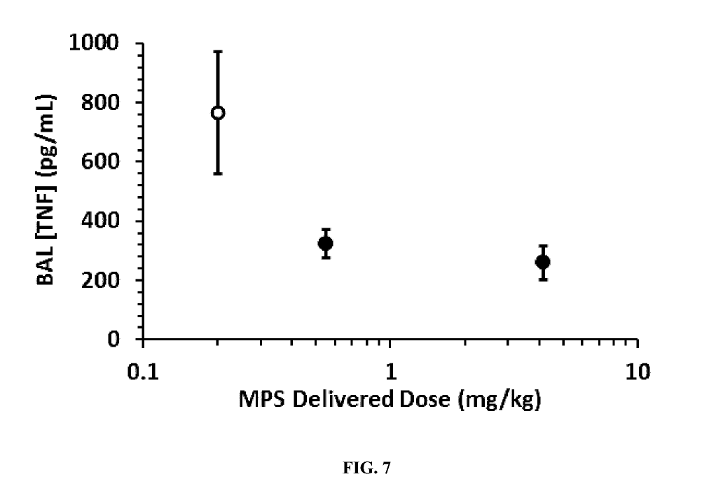

The graphed data are shown at FIG. 7. In FIG. 7, PBS data are represented by an open circle and MPS data are represented by the filled circles. The data indicate that inhaled MPS inhibits LPS-stimulated TNF production in the lungs of C57BL/6 mice.

Using the above-described methods, the ability of the compounds of the invention to decrease lipopolysaccharide (LPS)-stimulated TNF-α production in the lungs of C57BL/6 mice can be studied.

### Example 8

### Inhibition of Human Inosine 5′-Monophosphate Dehydrogenase

Human Inosine 5′-Monophosphate Dehydrogenase 1 (IMPDH) catalyzes the nicotinamide adenine dinucleotide (NAD)-dependent oxidation of inosine-5′-monophosphate (IMP) to xanthosine-5′-monophosphate (XMP), which is the committed step in de novo guanosine nucleotide biosynthesis. B and T lymphocytes depend on IMPDH activity to generate the guanosine nucleotide levels needed to initiate a proliferative response to mitogen or antigen. Inhibitors of IMPDH are known to have a strong immunosuppressive effect. Mycophenolic acid (MPA) is a potent non-competitive, reversible inhibitor of IMPDH.

A comparison of the ability of MPA and hexadecyl mycophenolate (mono-C₁₆MP) to inhibit IMPDH was undertaken.

IMPDH (R&D Systems 8904DH) was incubated with mycophenolic acid (MPA) and, separately, mono-C₁₆MP at concentrations ranging from about 0.015 to 15 μM. The IMPDH activity was determined using a BMR Service IMPDH assay kit (E-119).

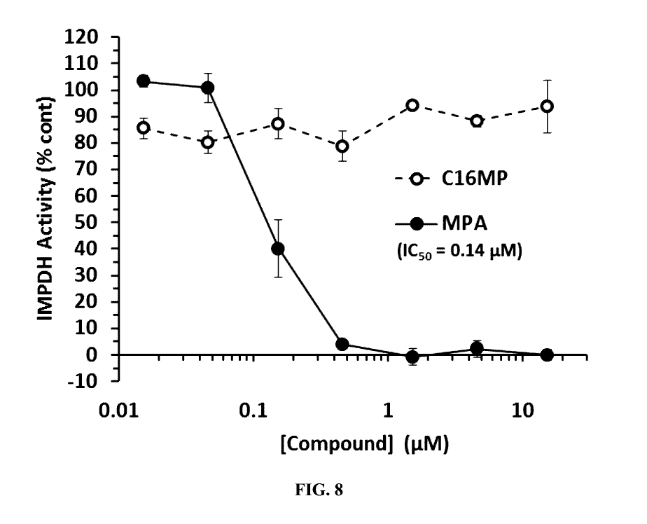

Inhibition of IMPDH was calculated as a percentage of IMPDH activity in the absence of MPA. As shown in FIG. 8, an WA-concentration dependent reduction in IMPDH activity was observed. The calculated IC₅₀ for MPA was 0.14 μM. On the other hand, mono-C₁₆MP does not inhibit IMPDH in a concentration-dependent manner.

Using the above-described methods, the ability of the compounds of the invention to inhibit IMPDH can be studied.

### Example 9

### Effect of Anti-Sarcoid Compound Formulations on Granuloma Formation in In Vivo Mouse Model of Sarcoidosis

The ability of compositions of the invention to decrease granuloma formation and improve lung histopatholgy can be tested in a mouse model of sarcoidosis. An exemplary mouse model of sarcoidosis is described in McCaskill et al., Am J Respir Cell Mol Biol., 2006 September; 35(3): 347-356, which is incorporated herein by reference for all purposes. Specifically, Propionibacterium acnes (PA) is a gram-positive anaerobic bacterium implicated as a putative etiologic agent of sarcoidosis. To induce sarcoidosis in mice, heat-killed PA can be injected intraperitoneally in C57BL/6 and/or BALB/c mice. Two weeks after intraperitoneal injection, PA-sensitized mice can be challenged with heat-killed PA (e.g. 0.5 mg: 0.05 mL of 10 mg/mL suspension) intratracheally. C57BL/6 and BALB/c mice sensitized and challenged with PBS (PBS/PBS) can be used as controls. Additionally, some mice can either be sensitized to PA but not challenged (intraperitoneal PA/intratracheal PBS), or nonsensitized but challenged (intraperitoneal PBS/intratracheal PA) to determine the impact of sensitization alone as well as challenge alone.

Formulations according to the invention can be administered to mice at various time points to determine the effect of formulations in improving pathophysiology of sarcoidosis, such as decrease in granuloma formation. For example, test and control formulations comprising a compound of Formula (I) can be administered at day 5, day 7, day 10, day 12, and/or day 14 post intra-peritoneal sensitization and day 2, day 5, day 7, day 10, day 14, day 21, and/or day 28 post intratracheal challenge.

McCaskill et al. have shown that mice challenged with PA developed a cellular immune response characterized by elevations in Th1 cytokines/chemokines, increased numbers of lymphocytes and macrophages in lung lavage fluid, and peribronchovascular granulomatous inflammation composed of T- and B-lymphocytes and epithelioid histiocytes, all of which resemble pathophysiology of sarcoidosis.

Mice can be sacrificed at specific time points and various pathological and immunological markers, such as those described in McCaskill et al., can be tested to determine the effect of the formulations of the invention on the pathophysiology of sarcoidosis. Additionally, mice can be followed for survival to determine the effect of formulations comprising a compound of Formula (I) on the survival.

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

Patents, patent applications, patent application publications, journal articles and protocols referenced herein are incorporated by reference in their entireties, for all purposes.

1. A compound represented by the formula: 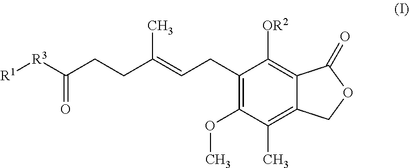
wherein
R¹ is C₈-C₂₀ alkyl,
R² is hydrogen, and
R³ is NH, O, or S;
or
a pharmaceutically acceptable salt thereof.

2. The compound of claim 1, wherein R³ is O, or a pharmaceutically acceptable salt thereof.

3.-13. (canceled)

14. The compound of claim 2, wherein R¹ is C₁₂-C₁₈ alkyl, or a pharmaceutically acceptable salt thereof.

15. The compound of claim 2, wherein R¹ is C₁₂-C₁₆ alkyl, or a pharmaceutically acceptable salt thereof.

16. The compound of claim 2, wherein R¹ is C₁₄-C₂₀ alkyl, or a pharmaceutically acceptable salt thereof.

17. The compound of claim 2, wherein R¹ is C₁₄-C₁₆ alkyl, or a pharmaceutically acceptable salt thereof.

18.-23. (canceled)

24. The compound of claim 2, wherein R¹ is C₁₆ alkyl, or a pharmaceutically acceptable salt thereof.

25.-83. (canceled)

84. A pharmaceutical composition comprising a pharmaceutically effective amount of a compound of claim 1, or pharmaceutically acceptable salt thereof, and a pharmaceutically acceptable excipient.

85.-119. (canceled)

120. A method of treating sarcoidosis in a patient in need thereof, comprising, administering a therapeutically effective amount of the pharmaceutical composition of claim 84 to the patient.

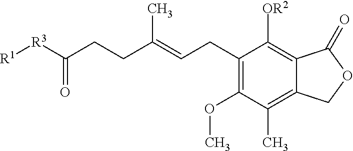

121.-213. (canceled)

214. The method of claim 120, wherein the sarcoidosis is pulmonary sarcoidosis.

215. The method of claim 120, wherein the administering is to the lungs of the patient.

216. The method of claim 120, wherein the administering is by inhalation.

217. The method of claim 216, wherein the administering is by a metered dose inhaler (MDI).

218. The method of claim 216, wherein the administering is by a dry powder inhaler (DPI).

219. The method of claim 216, wherein the administering is by a nebulizer.

220. The method of claim 216, wherein the administering is by a soft mist inhaler.

221.-223. (canceled)

224. The method of claim 120, wherein the patient is a cystic fibrosis patient.

225. The method of claim 120, wherein the patient has emphysema, chronic obstructive pulmonary disorder, or acute respiratory disorder.

226.-227. (canceled)

228. A kit comprising a composition of claim 84 and an inhalation delivery device.

229. The kit of claim 228, wherein the inhalation device is a metered dose inhaler (MDI), dry powder inhaler (DPI), a nebulizer or a soft mist inhaler.

230.-232. (canceled)
